SEQUENCE LISTING (116 sequences)

SEQ ID NO: 1 - protein, 38 aa, synthetic construct
  source 1..38: note = SEQ ID NO: 1 - CEBPB leucine zipper domain
LETQHKVLELTAENERLQKKVEQLSRELSTLRNLFKQL

SEQ ID NO: 2 - protein, 31 aa, synthetic construct
  source 1..31: note = SEQ ID NO: 2 - CEBPD leucine zipper domain
KLVELSAENEKLHQRVEQLTRDLAGLRQFFK

SEQ ID NO: 3 - protein, 7 aa, synthetic construct
  source 1..7: note = SEQ ID NO: 3 - CEBPB leucine zipper domain heptad B-1
KVLELTA

SEQ ID NO: 4 - protein, 7 aa, synthetic construct
  source 1..7: note = SEQ ID NO: 4 - CEBPB leucine zipper domain heptad B-2
ENERLQK

SEQ ID NO: 5 - protein, 7 aa, synthetic construct
  source 1..7: note = SEQ ID NO: 5 - CEBPB leucine zipper domain heptad B-3
KVEQLSR

SEQ ID NO: 6 - protein, 7 aa, synthetic construct
  source 1..7: note = SEQ ID NO: 6 - CEBPB leucine zipper domain heptad B-4
ELSTLRN

SEQ ID NO: 7 - protein, 7 aa, synthetic construct
  source 1..7: note = SEQ ID NO: 7 - CEBPD leucine zipper domain heptad D-1
KLVELSA

SEQ ID NO: 8 - protein, 7 aa, synthetic construct
  source 1..7: note = SEQ ID NO: 8 - CEBPD leucine zipper domain heptad D-2
ENEKLHQ

SEQ ID NO: 9 - protein, 7 aa, synthetic construct
  source 1..7: note = SEQ ID NO: 9 - CEBPD leucine zipper domain heptad D-3
RVEQLTR

SEQ ID NO: 10 - protein, 7 aa, synthetic construct
  source 1..7: note = SEQ ID NO: 10 - CEBPD leucine zipper domain heptad D-4
DLAGLRQ

SEQ ID NO: 11 - protein, 33 aa, synthetic construct
  source 1..33: note = SEQ ID NO: 11 - DNA binding domain of a CEBPB protein
KKTVDKHSDEYKIRRERNNIAVRKSRDKAKMRN

SEQ ID NO: 12 - protein, 35 aa, synthetic construct
  source 1..35: note = SEQ ID NO: 12 - DNA binding domain of a CEBPD protein
DRGSPEYRQRRERNNIAVRKSRDKAKRRNQEMQQK

SEQ ID NO: 13 - protein, 10 aa, synthetic construct
  source 1..10: note = SEQ ID NO: 13 - MYC
EQKLISEEDL

SEQ ID NO: 14 - protein, 30 aa, synthetic construct
  source 1..30: note = SEQ ID NO: 14 - three repeats of MYC
EQKLISEEDLEQKLISEEDLEQKLISEEDL

SEQ ID NO: 15 - protein, 15 aa, synthetic construct
  source 1..15: note = SEQ ID NO: 15 - AviTag
GLNDIFEAQKIEWHE

SEQ ID NO: 16 - protein, 26 aa, synthetic construct
  source 1..26: note = SEQ ID NO: 16 - Calmodulin- tag
KRRWKKNFIAVSAANRFKKISSSGAL

SEQ ID NO: 17 - protein, 6 aa, synthetic construct
  source 1..6: note = SEQ ID NO: 17 - polyglutamate tag
EEEEEE

SEQ ID NO: 18 - protein, 13 aa, synthetic construct
  source 1..13: note = SEQ ID NO: 18 - E-tag
GAPVPYPDPLEPR

SEQ ID NO: 19 - protein, 8 aa, synthetic construct
  source 1..8: note = SEQ ID NO: 19 - FLAG-tag
DYKDDDDK

SEQ ID NO: 20 - protein, 9 aa, synthetic construct
  source 1..9: note = SEQ ID NO: 20 - HA-tag
YPYDVPDYA

SEQ ID NO: 21 - protein, 6 aa, synthetic construct
  source 1..6: note = SEQ ID NO: 21 - 6 His-tag
HHHHHH

SEQ ID NO: 22 - protein, 10 aa, synthetic construct
  source 1..10: note = SEQ ID NO: 22 - 10 His-tag
HHHHHHHHHH

SEQ ID NO: 23 - protein, 18 aa, synthetic construct
  source 1..18: note = SEQ ID NO: 23 - NE-tag
TKENPRSNQEESYDDNES

SEQ ID NO: 24 - protein, 15 aa, synthetic construct
  source 1..15: note = SEQ ID NO: 24 - S-tag
KETAAAKFERQHMDS

SEQ ID NO: 25 - protein, 38 aa, synthetic construct
  source 1..38: note = SEQ ID NO: 25 - SBP-tag
MDEKTTGWRGGHVVEGLAGELEQLRARLEHHPQGQREP

SEQ ID NO: 26 - protein, 13 aa, synthetic construct
  source 1..13: note = SEQ ID NO: 26 - Softag 1
SLAELLNAGLGGS

SEQ ID NO: 27 - protein, 8 aa, synthetic construct
  source 1..8: note = SEQ ID NO: 27 - Softag 3
TQDPSRVG

SEQ ID NO: 28 - protein, 8 aa, synthetic construct
  source 1..8: note = SEQ ID NO: 28 - Strep-tag
WSHPQFEK

SEQ ID NO: 29 - protein, 6 aa, synthetic construct
  source 1..6: note = SEQ ID NO: 29 - TC tag
CCPGCC

SEQ ID NO: 30 - protein, 14 aa, synthetic construct
  source 1..14: note = SEQ ID NO: 30 - V5 tag
GKPIPNPLLGLDST

SEQ ID NO: 31 - protein, 11 aa, synthetic construct
  source 1..11: note = SEQ ID NO: 31 - VSV-tag
YTDIEMNRLGK

SEQ ID NO: 32 - protein, 8 aa, synthetic construct
  source 1..8: note = SEQ ID NO: 32 - Xpress tag
DLYDDDDK

SEQ ID NO: 33 - protein, 16 aa, synthetic construct
  source 1..16: note = SEQ ID NO: 33 - Isopeptag
TDKDMTITFTNKKDAE

SEQ ID NO: 34 - protein, 13 aa, synthetic construct
  source 1..13: note = SEQ ID NO: 34 - SpyTag
AHIVMVDAYKPTK

SEQ ID NO: 35 - protein, 12 aa, synthetic construct
  source 1..12: note = SEQ ID NO: 35 - SnoopTag
KLGDIEFIKVNK

SEQ ID NO: 36 - protein, 16 aa, synthetic construct
  source 1..16: note = SEQ ID NO: 36 - Penetratin 1
RQIKIWFQNRRMKWKK

SEQ ID NO: 37 - protein, 16 aa, synthetic construct
  source 1..16: note = SEQ ID NO: 37 - RL16
RRLRRLLRRLLRRLRR

SEQ ID NO: 38 - protein, 12 aa, synthetic construct
  source 1..12: note = SEQ ID NO: 38 - cell penetrating peptide from rabies virus
RVGRRRRRRRRR

SEQ ID NO: 39 - protein, 27 aa, synthetic construct
  source 1..27: note = SEQ ID NO: 39 - Transportan
GWTLNSAGYLLGKINLKALAALAKKIL

SEQ ID NO: 40 - protein, 16 aa, synthetic construct
  source 1..16: note = SEQ ID NO: 40 - pIs1
PVIRVWFQNKRCKDKK

SEQ ID NO: 41 - protein, 11 aa, synthetic construct
  source 1..11: note = SEQ ID NO: 41 - Tat fragment
YGRKKRRQRRR

SEQ ID NO: 42 - protein, 13 aa, synthetic construct
  source 1..13: note = SEQ ID NO: 42 - Tat fragment
GRKKRRQRRRPPQ

SEQ ID NO: 43 - protein, 16 aa, synthetic construct
  source 1..16: note = SEQ ID NO: 43 - pVEC
LLIILRRRIRKQAHAH

SEQ ID NO: 44 - protein, 27 aa, synthetic construct
  source 1..27: note = SEQ ID NO: 44 - MPS
GALFLGWLGAAGSTMGAWSQPKKKRKV

SEQ ID NO: 45 - protein, 18 aa, synthetic construct
  source 1..18: note = SEQ ID NO: 45 - MAP
KLALKLALKALKAALKLA

SEQ ID NO: 46 - protein, 16 aa, synthetic construct
  source 1..16: note = SEQ ID NO: 46 - penetratin
RQIKIFFQNRRMKFKK

SEQ ID NO: 47 - protein, 12 aa, synthetic construct
  source 1..12: note = SEQ ID NO: 47 - penetratin
RQIKIWFRKWKK

SEQ ID NO: 48 - protein, 46 aa, synthetic construct
  source 1..46: note = SEQ ID NO: 48 - Dpep
RQIKIWFQNRRMKWKKLVELSAENEKLHQRVEQLTRDLAGLRQFFK

SEQ ID NO: 49 - protein, 28 aa, synthetic construct
  source 1..28: note = SEQ ID NO: 49 - D-1234
KLVELSAENEKLHQRVEQLTRDLAGLRQ

SEQ ID NO: 50 - protein, 21 aa, synthetic construct
  source 1..21: note = SEQ ID NO: 50 - D-123
KLVELSAENEKLHQRVEQLTR

SEQ ID NO: 51 - protein, 36 aa, synthetic construct
  source 1..36: note = SEQ ID NO: 51 - CP-D-123
RQIKIWFQNRRMKWKKLVELSAENEKLHQRVEQLTR

SEQ ID NO: 52 - protein, 21 aa, synthetic construct
  source 1..21: note = SEQ ID NO: 52 - D-234
ENEKLHQRVEQLTRDLAGLRQ

SEQ ID NO: 53 - protein, 37 aa, synthetic construct
  source 1..37: note = SEQ ID NO: 53 - CP-D-234
RQIKIWFQNRRMKWKKENEKLHQRVEQLTRDLAGLRQ

SEQ ID NO: 54 - protein, 14 aa, synthetic construct
  source 1..14: note = SEQ ID NO: 54 - D-12
KLVELSAENEKLHQ

SEQ ID NO: 55 - protein, 29 aa, synthetic construct
  source 1..29: note = SEQ ID NO: 55 - CP-D-12
RQIKIWFQNRRMKWKKLVELSAENEKLHQ

SEQ ID NO: 56 - protein, 14 aa, synthetic construct
  source 1..14: note = SEQ ID NO: 56 - D-23
ENEKLHQRVEQLTR

SEQ ID NO: 57 - protein, 30 aa, synthetic construct
  source 1..30: note = SEQ ID NO: 57 - CP-D-23
RQIKIWFQNRRMKWKKENEKLHQRVEQLTR

SEQ ID NO: 58 - protein, 14 aa, synthetic construct
  source 1..14: note = SEQ ID NO: 58 - D-34
RVEQLTRDLAGLRQ

SEQ ID NO: 59 - protein, 30 aa, synthetic construct
  source 1..30: note = SEQ ID NO: 59 - CP-D-34
RQIKIWFQNRRMKWKKRVEQLTRDLAGLRQ

SEQ ID NO: 60 - protein, 22 aa, synthetic construct
  source 1..22: note = SEQ ID NO: 60 - CP-D-1
RQIKIWFQNRRMKWKKLVELSA

SEQ ID NO: 61 - protein, 23 aa, synthetic construct
  source 1..23: note = SEQ ID NO: 61 - CD-D-2
RQIKIWFQNRRMKWKKENEKLHQ

SEQ ID NO: 62 - protein, 23 aa, synthetic construct
  source 1..23: note = SEQ ID NO: 62 - CP-D-3
RQIKIWFQNRRMKWKKRVEQLTR

SEQ ID NO: 63 - protein, 23 aa, synthetic construct
  source 1..23: note = SEQ ID NO: 63 - CP-D-4
RQIKIWFQNRRMKWKKDLAGLRQ

SEQ ID NO: 64 - protein, 54 aa, synthetic construct
  source 1..54: note = SEQ ID NO: 64 - Bpep
RQIKIWFQNRRMKWKKLETQHKVLELTAENERLQKKVEQLSRELSTLRNLFKQL

SEQ ID NO: 65 - protein, 28 aa, synthetic construct
  source 1..28: note = SEQ ID NO: 65 - B-1234
KVLELTAENERLQKKVEQLSRELSTLRN

SEQ ID NO: 66 - protein, 43 aa, synthetic construct
  source 1..43: note = SEQ ID NO: 66 - CP-B-1234
RQIKIWFQNRRMKWKKVLELTAENERLQKKVEQLSRELSTLRN

SEQ ID NO: 67 - protein, 21 aa, synthetic construct
  source 1..21: note = SEQ ID NO: 67 - B-123
KVLELTAENERLQKKVEQLSR

SEQ ID NO: 68 - protein, 36 aa, synthetic construct
  source 1..36: note = SEQ ID NO: 68 - CP-B-123
RQIKIWFQNRRMKWKKVLELTAENERLQKKVEQLSR

SEQ ID NO: 69 - protein, 21 aa, synthetic construct
  source 1..21: note = SEQ ID NO: 69 - B-234
ENERLQKKVEQLSRELSTLRN

SEQ ID NO: 70 - protein, 37 aa, synthetic construct
  source 1..37: note = SEQ ID NO: 70 - CP-B-234
RQIKIWFQNRRMKWKKENERLQKKVEQLSRELSTLRN

SEQ ID NO: 71 - protein, 14 aa, synthetic construct
  source 1..14: note = SEQ ID NO: 71 - B-12
KVLELTAENERLQK

SEQ ID NO: 72 - protein, 29 aa, synthetic construct
  source 1..29: note = SEQ ID NO: 72 - CP-B-12
RQIKIWFQNRRMKWKKVLELTAENERLQK

SEQ ID NO: 73 - protein, 14 aa, synthetic construct
  source 1..14: note = SEQ ID NO: 73 - B-23
ENERLQKKVEQLSR

SEQ ID NO: 74 - protein, 30 aa, synthetic construct
  source 1..30: note = SEQ ID NO: 74 - CP-B-23
RQIKIWFQNRRMKWKKENERLQKKVEQLSR

SEQ ID NO: 75 - protein, 14 aa, synthetic construct
  source 1..14: note = SEQ ID NO: 75 - B-34
KVEQLSRELSTLRN

SEQ ID NO: 76 - protein, 29 aa, synthetic construct
  source 1..29: note = SEQ ID NO: 76 - CP-B-34
RQIKIWFQNRRMKWKKVEQLSRELSTLRN

SEQ ID NO: 77 - protein, 22 aa, synthetic construct
  source 1..22: note = SEQ ID NO: 77 - CP-B-1
RQIKIWFQNRRMKWKKVLELTA

SEQ ID NO: 78 - protein, 23 aa, synthetic construct
  source 1..23: note = SEQ ID NO: 78 - CP-B-2
RQIKIWFQNRRMKWKKENERLQK

SEQ ID NO: 79 - protein, 22 aa, synthetic construct
  source 1..22: note = SEQ ID NO: 79 - CP-B-3
RQIKIWFQNRRMKWKKVEQLSR

SEQ ID NO: 80 - protein, 23 aa, synthetic construct
  source 1..23: note = SEQ ID NO: 80 - CP-B-4
RQIKIWFQNRRMKWKKELSTLRN

SEQ ID NO: 81 - protein, 21 aa, synthetic construct
  source 1..21: note = SEQ ID NO: 81 - D-123WWK
KLVELSAENEKLHQKVEQLKR

SEQ ID NO: 82 - protein, 21 aa, synthetic construct
  source 1..21: note = SEQ ID NO: 82 - D-123WWE
KLVELSAENEKLHQEVEQLER

SEQ ID NO: 83 - protein, 21 aa, synthetic construct
  source 1..21: note = SEQ ID NO: 83 - D-123WKW
KLVELSAKNEKLKQRVEQLTR

SEQ ID NO: 84 - protein, 21 aa, synthetic construct
  source 1..21: note = SEQ ID NO: 84 - D-123WEW
KLVELSAENEKLEQRVEQLTR

SEQ ID NO: 85 - protein, 21 aa, synthetic construct
  source 1..21: note = SEQ ID NO: 85 - D-123KWW
KLVELKAENEKLHQRVEQLTR

SEQ ID NO: 86 - protein, 21 aa, synthetic construct
  source 1..21: note = SEQ ID NO: 86 - D-123EWW
ELVELEAENEKLHQRVEQLTR

SEQ ID NO: 87 - protein, 21 aa, synthetic construct
  source 1..21: note = SEQ ID NO: 87 - D-123WKK
KLVELSAKNEKLKQKVEQLKR

SEQ ID NO: 88 - protein, 21 aa, synthetic construct
  source 1..21: note = SEQ ID NO: 88 - D-123WEE
KLVELSAENEKLEQEVEQLER

SEQ ID NO: 89 - protein, 21 aa, synthetic construct
KLVELKAENEKLHQKVEQLKR

SEQ ID NO: 90 - protein, 21 aa, synthetic construct
  source 1..21: note = SEQ ID NO: 90 - D-123EWE
ELVELEAENEKLHQEVEQLER

SEQ ID NO: 91 - protein, 21 aa, synthetic construct
  source 1..21: note = SEQ ID NO: 91 - D-123KKW
KLVELKAKNEKLKQRVEQLTR

SEQ ID NO: 92 - protein, 21 aa, synthetic construct
  source 1..21: note = SEQ ID NO: 92 - D-123EEW
ELVELEAENEKLEQRVEQLTR

SEQ ID NO: 93 - protein, 21 aa, synthetic construct
  source 1..21: note = SEQ ID NO: 93 - D-123EKW
ELVELEAKNEKLKQRVEQLTR

SEQ ID NO: 94 - protein, 21 aa, synthetic construct
  source 1..21: note = SEQ ID NO: 94 - D-123EWK
ELVELEAENEKLHQKVEQLKR

SEQ ID NO: 95 - protein, 21 aa, synthetic construct
  source 1..21: note = SEQ ID NO: 95 - D-123WEK
KLVELSAENEKLEQKVEQLKR

SEQ ID NO: 96 - protein, 21 aa, synthetic construct
  source 1..21: note = SEQ ID NO: 96 - D-123WKE
KLVELSAKNEKLKQEVEQLER

SEQ ID NO: 97 - protein, 21 aa, synthetic construct
  source 1..21: note = SEQ ID NO: 97 - D-123KWE
KLVELKAENEKLHQEVEQLER

SEQ ID NO: 98 - protein, 21 aa, synthetic construct
  source 1..21: note = SEQ ID NO: 98 - D-123KEW
KLVELKAENEKLEQRVEQLTR

SEQ ID NO: 99 - protein, 21 aa, synthetic construct
  source 1..21: note = SEQ ID NO: 99 - D-123KEE
KLVELKAENEKLEQEVEQLER

SEQ ID NO: 100 - protein, 21 aa, synthetic construct
  source 1..21: note = SEQ ID NO: 100 - D-123EKE
ELVELEAKNEKLKQEVEQLER

SEQ ID NO: 101 - protein, 21 aa, synthetic construct
  source 1..21: note = SEQ ID NO: 101 - D-123EEK
ELVELEAENEKLEQKVEQLKR

SEQ ID NO: 102 - protein, 21 aa, synthetic construct
  source 1..21: note = SEQ ID NO: 102 - D-123EEE
ELVELEAENEKLEQEVEQLER

SEQ ID NO: 103 - protein, 21 aa, synthetic construct
  source 1..21: note = SEQ ID NO: 103 - D-123KKK
KLVELKAKNEKLKQKVEQLKR

SEQ ID NO: 104 - protein, 21 aa, synthetic construct
  source 1..21: note = SEQ ID NO: 104 - D-123KKE
KLVELKAKNEKLKQEVEQLER

SEQ ID NO: 105 - protein, 21 aa, synthetic construct
  source 1..21: note = SEQ ID NO: 105 - D-123KEK
KLVELKAENEKLEQKVEQLKR

SEQ ID NO: 106 - protein, 21 aa, synthetic construct
  source 1..21: note = SEQ ID NO: 106 - D-123EKK
ELVELEAKNEKLKQKVEQLKR

SEQ ID NO: 107 - protein, 21 aa, synthetic construct
  source 1..21: note = SEQ ID NO: 107 - ATF5 segment
ECQGLEARNRELKERAESVER

SEQ ID NO: 108 - protein, 21 aa, synthetic construct
  source 1..21: note = SEQ ID NO: 108 - CEBPD segment
KLVELSAENEKLHQRVEQLTR

SEQ ID NO: 109 - protein, 21 aa, synthetic construct
  source 1..21: note = SEQ ID NO: 109 - CEBPB segment
KVLELTAENERLQKKVEQLSR

SEQ ID NO: 110 - protein, 36 aa, synthetic construct
  source 1..36: note = SEQ ID NO: 110 - CP-D-123EKE
RQIKIWFQNRRMKWKELVELEAKNEKLKQEVEQLER

SEQ ID NO: 111 - protein, 21 aa, synthetic construct
  source 1..21: note = SEQ ID NO: 111 - D-123-mut
KLVEGSAENEKGHQRVEQGTR

SEQ ID NO: 112 - protein, 21 aa, synthetic construct
  source 1..21: note = SEQ ID NO: 112 - D-123EKE-mut
ELVEGEAKNEKGKQEVEQGER

SEQ ID NO: 113 - protein, 21 aa, synthetic construct
  source 1..21: note = SEQ ID NO: 113 - B-123-mut
KVLEGTAENERGQKKVEQGSR

SEQ ID NO: 114 - protein, 21 aa, synthetic construct
  source 1..21: note = SEQ ID NO: 114 - B-123EKE
EVLELEAKNERLKKEVEQLER

SEQ ID NO: 115 - protein, 28 aa, synthetic construct
  source 1..28: note = SEQ ID NO: 115 - B-mut
KVLEGTAENERGQKKVEQGSRELSTGRN

SEQ ID NO: 116 - protein, 28 aa, synthetic construct
  source 1..28: note = SEQ ID NO: 116 - D-mut
KLVEGSAENEKGHQRVEQGTRDLAGGRQ

(19) United States
(12) Patent Application Publication
(10) Pub. No.: US 2025/0223328 A1
(43) Pub. Date: Jul. 10, 2025

(54) TRUNCATED DOMINANT NEGATIVE CEBPB AND CEBPD PROTEINS AND METHODS OF USE FOR DECREASING VIABILITY OF NEOPLASTIC CELLS

(71) Applicants: The Trustees of Columbia University in the City of New York, New York, NY (US); The Regents of the University of California, Oakland, CA (US)

(72) Inventors: Lloyd A. GREENE, Larchmont, NY (US); Qing ZHOU, New York, NY (US); James M. ANGELASTRO, Davis, CA (US)

(21) Appl. No.: 19/091,382

(22) Filed: Mar. 26, 2025

Related U.S. Application Data
(63) Continuation of application No. PCT/US2023/077390, filed on Oct. 20, 2023.
(60) Provisional application No. 63/418,370, filed on Oct. 21, 2022.

(51) Int. Cl. C07K 14/47; A61K 38/00; A61K 45/06; A61P 35/00

(52) U.S. Cl. CPC C07K 14/4747; A61K 45/06; A61P 35/00; A61K 38/00; C07K 2319/10; C07K 2319/73

(57) ABSTRACT

Truncated dominant negative forms of CEBPB and CEBPD, and cell-penetrating forms thereof are described. Methods for using the truncated dominant negative forms of CEBPB and CEBPD proteins, and cell-penetrating forms thereof, for decreasing viability of neoplastic cells and treating cancer in a subject are also described.

This application claims the benefit of U.S. Provisional Application No. 63/418,370, filed Oct. 21, 2022, the content of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### STATEMENT REGARDING FEDERALLY SPONSORED RESEARCH OR DEVELOPMENT

This invention was made with government support under NS072050 awarded by the National Institutes of Health. The government has certain rights in the invention.

### REFERENCE TO SEQUENCE LISTING

This application incorporates-by-reference nucleotide sequences which are present in the file named “231020_92305-A-PCT_Sequence_Listing_AWG”, which is 121 kilobytes in size, and which was created on Oct. 19, 2023 in the IBM-PC machine format, having an operating system compatibility with MS-Windows, which is contained in the xml file filed Mar. 26, 2025 as part of this application.

### TECHNICAL FIELD

The present disclosure relates to proteins and methods for decreasing viability of neoplastic cells, including tumor cells. In particular, the present disclosure relates to truncated dominant negative (DN) forms of CCAAT/enhancer-binding protein beta (CEBPB) and CCAAT/enhancer-binding protein delta (CEBPD) proteins, cell-penetrating forms of these peptides, and methods of use thereof for decreasing viability of neoplastic cells and treating cancer in a subject.

### BACKGROUND OF THE INVENTION

Approximately one million people are diagnosed with cancer each year, and many millions of people in the United States of all ages are currently living with some form of cancer. Despite intensive research, discovery of new therapeutic targets and development of new drugs for treating cancer remains challenging.

### BRIEF SUMMARY OF THE INVENTION

The subject invention provides a dominant negative protein comprising a leucine zipper domain having 90% or greater homology to any one of SEQ ID NOs: 7-10, 50, 52, 54, 56, 58, or 81-106.

The subject invention also provides a dominant negative protein comprising a leucine zipper domain having 90% or greater homology to any one of SEQ ID NOs: 3-6, 65, 67, 69, 71, 73, 75, or 77-80.

The subject invention also provides a non-naturally occurring composition comprising a peptide, wherein the peptide comprises a CCAAT/enhancer-binding protein delta (CEBPD) leucine zipper domain comprising one to four CEBPD repeat portions, wherein each CEBPD repeat portion has at least 70% sequence identity to any one of SEQ ID NOs: 7-10.

The subject invention also provides a non-naturally occurring composition comprising a peptide, wherein the peptide comprises a CCAAT/enhancer-binding protein beta (CEBPB) leucine zipper domain comprising one to four CEBPB repeat portions, wherein each CEBPB repeat portion has at least 70% sequence identity to any one of SEQ ID NOs: 3-6.

### BRIEF DESCRIPTION OF THE DRAWINGS

For a more complete understanding of the present disclosure and the associated features and advantages, reference is now made to the following description, taken in conjunction with the accompanying drawings, which are not to scale, and in which:

FIG. 1 is a graph reporting exemplary data showing the proportion of HCT116 cells with apoptotic nuclei following transfection with vectors expressing D-mut, D-1234, D-123, or D-234.

FIG. 2 is a graph reporting exemplary data showing the proportion of HCT116 cells with apoptotic nuclei following transfection with vectors expressing D-mut, D-1234, D-12, D-23, or D-34.

FIGS. 3A-3E are graphs reporting exemplary data showing relative cell numbers after 6 days of treatment with the indicated concentrations of CP-D-123. The experiments were performed in T98G (FIG. 3A), MDA-MB-231 (FIG. 3B), A375 (FIG. 3C), Shep21N (FIG. 3D), and CHLA255 (FIG. 3E) cell lines.

FIGS. 4A-4E are graphs reporting exemplary data showing relative cell numbers after treatment with the indicated concentrations of CP-D-123, CP-D-234, and Bpep (at 20 μM in T98G, MDA-MB-231, A375 only). The experiments were performed in T98G (FIG. 4A), MDA-MB-231 (FIG. 4B), A375 (FIG. 4C), Shep21N (FIG. 4D), and CHLA255 (FIG. 4E) cell lines.

FIGS. 5-7 are graphs reporting exemplary data showing mean tumor volumes (mm³) (FIG. 5), mean animal weight (grams) (FIG. 6), and the number of surviving animals (FIG. 7) for A375 xenograft mice treated intraperitoneally three times per week with either vehicle (PBS) or CP-D-123 (10 mg/kg).

FIG. 8 shows an exemplary illustration of an end view of two interacting leucine helices in a coiled-coil structure.

FIGS. 9A-9D show exemplary illustrations of each of the four heptad repeats of a D-1234 homodimer. A CEBPD leucine zipper domain having the amino acid sequence KLVELSAENEKLHQRVEQLTRDLAGLRQFFK (SEQ ID NO: 2) including positions a to g is listed beneath the diagram in each of FIGS. 9A-9D. Helix 1 is shown in FIG. 9A (D-1). Helix 2 is shown in FIG. 9B (D-2). Helix 3 is shown in FIG. 9C (D-3). Helix 4 is shown in FIG. 9D (D-4).

FIG. 10 shows exemplary alignments between the sequences of the D-123 peptide with itself and with corresponding segments from ATF5, CEBPD, and CEBPB. Attractive charge interactions are shown by lines with double arrows.

FIGS. 11A-11D shows alignments between the sequences of the D-123EWE (FIG. 11A), D-123EKE (FIG. 11B), D-123KEW (FIG. 11C), and D-123KEK (FIG. 11D) peptides with themselves and with corresponding segments from ATF5, CEBPD, and CEBPB. Attractive charge interactions are shown by lines with double arrows. Repulsive charge interactions are shown lines with circles at each end.

FIG. 12 is a graph reporting exemplary data showing the proportion of HCT116 cells with apoptotic nuclei following transfection with an empty vector and vectors expressing D-123, D-123EWE, or D-123EKE.

FIG. 13 is a graph reporting exemplary data showing relative MDA-MB-231 cell numbers after 6 days of treatment with the indicated concentrations of Dpep or CP-D-123EKE.

FIGS. 14A-14B are graphs reporting exemplary data showing relative T98G (FIG. 14A) and A375 (FIG. 14B) cell numbers after 6 days of treatment with the indicated concentrations of CP-D-123EKE.

FIGS. 15A-15D are examples of models created by PatchMAN (Khramushin et al., “Matching protein surface structural patches for high-resolution blind peptide docking” PNAS 2022 May; 119 (18) c2121153119) for docking of D-123 and D-123EKE with hCEBPD and hCEBPB. FIG. 15A shows PatchMAN analysis for docking of D-123 (SEQ ID NO: 50) with hCEBPD. FIG. 15B shows PatchMAN analysis for docking of D-123EKE (SEQ ID NO: 100) with hCEBPD. FIG. 15C shows PatchMAN analysis for docking of D-123-mut (SEQ ID NO: 111) with hCEBPD. FIG. 15D shows PatchMAN analysis for docking of D-123 (SEQ ID NO: 50) with hCEBPB.

FIGS. 16A-16B show calculated relative binding scores of D-123 (SEQ ID NO: 50), D-123-mut (SEQ ID NO: 111), D-123EKE (SEQ ID NO: 100), and D-123EKE-mut (SEQ ID NO: 112) to CEBPD, CEBPB and ATF5 (FIG. 16A); and B-123 (SEQ ID NO: 67), B-123-mut (SEQ ID NO: 113), and B-123EKE (SEQ ID NO: 114) to CEBPD, CEBPB and ATF5 (FIG. 16B). All scores normalized to binding by D-123 (without penetratin domain) to CEBPD. Data predict poor binding by mutant peptides and enhanced binding by D-123EKE and B-123EKE. Note enhanced binding of B-123 to ATF5. Binding scores calculated by PatchMAN analysis.

### DETAILED DESCRIPTION OF THE INVENTION

In the following description, details are set forth by way of example to facilitate discussion of the disclosed subject matter. It should be apparent to a person of ordinary skill in the art, however, that the disclosed implementations are exemplary and not exhaustive of all possible implementations.

The present disclosure relates to truncated dominant negative (DN) proteins and methods for inhibiting activity or viability of neoplastic cells, while having no, or a comparatively limited, effect on the activity or viability of non-neoplastic cells. In particular, the present disclosure relates to truncated dominant negative forms of CCAAT/enhancer-binding protein beta (CEBPB) and CCAAT/enhancer-binding protein delta (CEBPD), and cell-penetrating forms thereof. The present disclosure also relates to methods for using the truncated dominant negative forms of CEBPB and CEBPD, and cell-penetrating forms thereof, for inhibiting neoplastic cells and for treating cancer in a subject.

The truncated DN proteins of the present disclosure have several advantages when compared to longer DN proteins. Shorter peptides can be synthesized at a lower cost and with fewer errors, and shorter peptides are potentially easier to optimize for in vivo stability and efficacy. In addition, shorter peptides are believed to possess greater mobility in vivo, with better penetration into tissues and tumors, and shorter peptides have the potential to bind their targets with higher specificity and avidity. Accordingly, the truncated DN proteins of the present disclosure allow for cancer therapeutics with increased efficacy with fewer potential side effects and at lower cost.

Approximately one million people are diagnosed with cancer each year, and many millions of Americans of all ages are currently living with some form of cancer. At some time during the course of their lifetime, one out of every two American men and one out of every three American women will be diagnosed with some form of cancer. Despite intensive research, discovery of new therapeutic targets and development of new drugs for treating cancer remains challenging.

### CEBPB

In some implementations, the present disclosure relates to a truncated dominant negative CEBPB protein or a truncated dominant negative CEBPD protein.

The term “CEBPB protein” refers to CCAAT/enhancer-binding protein beta protein. CEBPB is a protein that in humans is encoded by the CEBPB gene (Szpirer C, Riviere M, Cortese R, Nakamura T, Islam M Q, Levan G, Szpirer J (July 1992). Genomics. 13 (2): 293-300; Cao Z, Umek R M, Mcknight S L (October 1991). Genes Dev. 5 (9): 1538-52). CEBPB is a bZIP transcription factor that can bind as a homodimer to certain DNA regulatory regions. There is also evidence that CEBPB can also form heterodimers with ATF5 via a leucine zipper: leucine zipper interaction (Sun, et al., Mol. Cancer Res. 2020 February; 18 (2): 216-228) and the leucine zipper transcription factors proteins ATF4 (Mann, et al., Genome Res. 2013 June; 23 (6): 988-97) and CEBPD (Wu, et al., Mol. Cell Biol. 1996 August; 16 (8): 4128-36). Translation of the C/EBP mRNA from different initiation codons leads to the synthesis of two transcriptional activators (LAP-1 and 2) and a transcriptional repressor (LIP). The LIP/LAP ratio is a critical factor in C/EBP-mediated gene transcription (Li et al., 2008, Journal Biological Chemistry, 283:22443). For example, CEBPB is important in the regulation of genes involved in immune and inflammatory responses and has been shown to bind to the IL-1 response element in the IL-6 gene, as well as to regulatory regions of several acute-phase and cytokine genes. In addition, CEBPB can bind the promoter and upstream element and stimulate the expression of the collagen type I gene. CEBPB is capable of increasing the expression of several target genes. Among them, some have specific role in the nervous system such as the preprotachykinin-1 gene, giving rise to substance P and neurokinin A and the choline acetyltransferase responsible for the biosynthesis of the important neurotransmitter acetylcholine. Other targets include genes coding for cytokines such as IL-6, IL-4, IL-5, and TNF-alpha. Genes coding for transporter proteins that confer multidrug resistance to the cells have also been found to be activated by CEBPB. Such genes include ABCC2 and ABCB1.

As used herein, “CEBPB” includes both an “CEBPB protein” and an “CEBPB analogue”. Unless otherwise indicated, “protein” shall include a protein, protein domain, polypeptide, or peptide, and any fragment thereof. For example, the CEBPB protein can have the amino acid sequence set forth in NCBI Accession No. NP_001272808.1 (human isoform c) or NCBI Accession No. NP_001272807.1 (human isoform b) or NCBI Accession No. NP_005185.2 (human isoform a), including conservative substitutions thereof. As used herein, “conservative substitutions” are those amino acid substitutions which are functionally equivalent to a substituted amino acid residue, either because they have similar polarity or steric arrangement, or because they belong to the same class as the substituted residue (e.g., hydrophobic, acidic, or basic).

A “CEBPB analogue”, as used herein, is a functional variant of the CEBPB protein, having CEBPB biological activity, such as ability of the CEBPB analogue's leucine zipper domain to bind to the protein binding partners of CEBPB, that has 60% or greater, 70% or greater or 80% or greater or 90% or greater or 95% or greater amino acid sequence homology or sequence identity with the CEBPB protein.

Persons of ordinary skill in the art will understand that the numbering of amino acid residues in CEBPB may be different than that set forth herein, or may contain certain conservative amino acid substitutions that produce the same CEBPB activity as that described herein. Corresponding amino acids and conservative substitutions in other isoforms or analogues are easily identified by visually inspecting the relevant amino acid sequences, or by using homology software programs identifiable by skilled persons.

### CEBPD

The term “CEBPD protein” refers to CCAAT/enhancer-binding protein delta protein. CEBPD is a protein that in humans is encoded by the CEBPD gene (Williams S C, Cantwell C A, Johnson P F (September 1991). “A family of C/EBP-related proteins capable of forming covalently linked leucine zipper dimers in vitro”. Genes & Development. 5 (9): 1553-67; Cao Z, Umck R M, Mcknight S L (October 1991). Genes Dev. 5 (9): 1538-52). CEBPD is a bZIP transcription factor which can bind as a homodimer to certain DNA regulatory regions. It can also form heterodimers with the related bZIP proteins CEBP, and ATF5. CEBPD protein is important in the regulation of genes involved in immune and inflammatory responses and may be involved in the regulation of genes associated with activation and/or differentiation of macrophages. CEBPD is involved in regulation of apoptosis and cell proliferation.

As used herein, “CEBPD” includes both an “CEBPD protein” and an “CEBPD analogue”. For example, the CEBPD protein can have the amino acid sequence set forth in NCBI Accession No. NP_005186 (human), including conservative substitutions thereof.

A “CEBPD analogue”, as used herein, is a functional variant of the CEBPD protein, having CEBPD biological activity, such as ability of the CEBPD analogue's leucine zipper domain to bind to the protein binding partners of CEBPD, that has 60% or greater, 70% or greater or 80% or greater or 90% or greater or 95% or greater amino acid sequence homology or sequence identity with the CEBPD protein.

Persons of ordinary skill in the art will understand that the numbering of amino acid residues in CEBPD may be different than that set forth herein, may contain certain conservative amino acid substitutions that produce the same CEBPD activity as that described herein. Corresponding amino acids and conservative substitutions in other isoforms or analogues are easily identified by visually inspecting the relevant amino acid sequences, or by using homology software programs identifiable by skilled persons.

CEBPB and CEBPD are recognized as oncogenic drivers that are downstream of pathways such as those involving Ras and BRAF. As such, they have been implicated in oncogenic transformation, proliferation, survival, invasiveness, resistance to treatment, and poor clinical outcome for a range of tumor types including blood cell, breast, skin, prostate and brain (Tregnago et al. (2016) Leukemia 30, 1887-1896; Wang et al. (2017) Clin Cancer Res 23, 503-513; Banerjee et al. (2016) Free Radic Biol Med 99, 296-307; Wang, et al. (2015) Oncotarget 6, 31069-31084; Balamurugan and Sterneck (2013) Int J Biol Sci 9, 917-933; Wu et al. (2011) J Biol Chem 286, 28662-28670; Balamurugan et al. (2010) EMBO J 29, 4106-4117; Liu et al. (2018) Nat Commun 9, 1739; Huang et al. (2018) Cancer Lett 421, 63-72; Li et al. (2018) Neoplasma 65, 34-41; Ji et al. (2018) Genet Test Mol Biomarkers 22, 5-10; Yin et al. (2017) Cancer Res 77, 4973-4984; Cao et al. (2017) Exp Ther Med 14, 1554-1560; Gardiner et al. (2017) Oncotarget 8, 26013-26026; Aguilar-Morante et al. (2011) Neuroscience 176, 110-119; Marigo et al. (2010) Immunity 32, 790-802; Carro et al. (2010) Nature 463, 318-325; Kim et al. (2009) Prostate 69, 1435-1447; Pal et al. (2009) Blood 114, 3890-3898; Shuman et al. (2004) Mol Cell Biol 24, 7380-7391; Duprez (2004) Cell Cycle 3, 389-390; Grimm and Rosen (2003) J Mammary Gland Biol Neoplasia 8, 191-204; Bundy and Scaly (2003) Oncogene 22, 869-883; Zhu, S., Yoon, K., Sterneck, E., Johnson, P. F., and Smart, R. C. (2002) CCAAT/enhancer binding protein-beta is a mediator of keratinocyte survival and skin tumorigenesis involving oncogenic Ras signaling. Proc Natl Acad Sci USA 99, 207-212). Significantly, many of these characteristics are suppressed by experimental CEBPB/D loss-of-function. Such activities have also been linked to the roles of CEBPB/D as regulators of the “mesenchymal transition”. For example, in gliomas, CEBPB and CEBPD have been identified as 2 of the 4 “master regulators” of the mesenchymal transition responsible for many negative properties of these tumors (Carro et al. (2010) Nature 463, 318-325; Califano and Alvarez (2017) Nat Rev Cancer 17, 116-130). In addition, CEBPB has been described as “a critical regulator of the immunosuppressive environment created by growing cancers” (Marigo et al. (2010) Immunity 32, 790-802).

In general, the terms “bind” or “binding” as used herein in connection with inter-molecular interactions such as those between proteins, or domains or motifs thereof, or between proteins and other molecules, such as DNA, refers to the connecting or uniting, at least for a time, of two or more molecules by a bond, link, force or tie in order to keep two or more molecules together, at least for a time. Exemplary bonds include without limitation covalent bond, ionic bond, van der Waals interactions, hydrogen bonds, and other bonds identifiable by a skilled person. In some instances, binding of a first molecule, such as the DN proteins described herein, with a second molecule, such as a binding partner of the DN protein, can result in sequestering the second molecule, thus providing a type of inhibition of the second molecule.

The term “leucine zipper” or “leucine zipper domain” refers to a three-dimensional structural motif, or the peptide sequence that composes such a motif, found in some proteins including CEBPB and CEBPD, as described herein. Leucine zippers are included in dimerization domains of the bZIP (basic-region leucine zipper) class of eukaryotic transcription factors.

The mechanism of transcriptional regulation by bZIP proteins typically occurs through binding affinity for ACGT motifs, which include CACGTG (G box), GACGTC (C box), TACGTA (A box), AACGTT (T box), and a GCN4 motif, namely TGA (G/C) TCA. A small number of bZIP factors such as OsOBFI can also recognize palindromic sequences. However, the others, including LIP19, OsZIP-2a, and OsZIP-2b, among others, do not bind to DNA sequences. Instead, these bZIP proteins form heterodimers with other bZIPs to regulate transcriptional activities.

The leucine zipper is a common three-dimensional structural motif in proteins. Leucine zipper domains typically contain a repeated 7 residue motif (heptad). Amino acid residues with a heptad are represented by the letters a through g. The amino acids in the a and d position are typically hydrophobic residues and frequently leucine, isoleucine, or valine. Leucine residues frequently occur in the d position. The amino acids in the b, c, e, f, and g positions are commonly polar residues.

Leucine zipper domains typically fold into a helical structure. Two leucine zipper domains may dimerize to form a coiled-coil structure where the a and d residues of a first domain interact with a′ and d′ residues of a second domain to form a hydrophobic core. Leucine zippers may form homodimers, where the two interacting leucine zipper domains have the same amino acid sequence or heterodimers, where the two interacting leucine zipper domains have different amino acid sequences. In some embodiments, a leucine zipper domain comprises 1 to 4 heptad repeat motifs.

In general, the terms “dominant negative” or “DN” as used herein refer to a protein variant capable of blocking the function of the normal, wild-type protein within the same cell. For example, in some instances, dominant negative activity may occur if the protein variant is capable of binding, or otherwise interacting, with the same cellular components (such as protein binding partners) as the wild-type protein, but blocking one or more aspects of the function of the wild type protein. In particular, the terms “dominant negative” or “DN” as used herein refers to a protein that has been modified so that it interacts with the normal binding partners (such as protein binding partners) for that protein, but is lacking the activity that would normally be present when it forms such interactions. In various implementations described herein, the dominant negative activity is due to the modification of or deletion of sequences from the WT protein to provide the DN protein. For example, the DN forms of CEBPB and CEBPD described herein retain the capacity to bind to the binding partners of the WT forms of ATF5, CEBPB and CEBPD for example through their native leucine zipper domains. However, the homodimers or heterodimers that are formed that include a DN CEBPB or DN CEBPD, are non-functional and have the effect of sequestering their binding partners so that they cannot perform their normal cellular functions.

The terms “truncated dominant negative” or “truncated DN” as used herein refer to a protein variant capable of blocking the function of the normal, wild-type protein within the same cell that has an amino acid sequence length that is less than the amino acid sequence length of a parent dominant negative protein. Preferably, a truncated DN protein includes a leucine zipper domain that is fewer amino acids in length than a parent DN protein. More preferably, a truncated DN protein includes a leucine zipper domain that includes fewer heptad repeats than a parent DN protein leucine zipper domain.

As described herein, the parent DN CEBPB protein consists essentially of a CEBPB leucine zipper domain capable of binding to binding partners of CEBPB. For example, in various implementations, the parent CEBPB leucine zipper domain can have an amino acid sequence LETQHKVLELTAENERLQKKVEQLSRELSTLRNLFKQL (SEQ ID NO: 1).

The parent CEBPB leucine zipper domain of SEQ ID NO: 1 includes at least the heptad repeats shown in Table 1. Using the nomenclature described above, each of the listed heptad repeats begins with a residue in the g position followed sequentially by residues in the a through f positions.

TABLE 1
Amino acid sequences of DN CEBPB constructs

| Construct | Amino acid sequence |
|---|---|
| B-1 | KVLELTA (SEQ ID NO: 3) |
| B-2 | ENERLQK (SEQ ID NO: 4) |
| B-3 | KVEQLSR (SEQ ID NO: 5) |
| B-4 | ELSTLRN (SEQ ID NO: 6) |

Truncated DN CEBPB proteins may include one, two, three, or all four of the heptad repeats listed in Table 1. Example 1 and Table 4 provide exemplary truncated DN CEBPB proteins.

The present disclosure also contemplates and encompasses variants of truncated DN CEBPB proteins, wherein the leucine zipper domain can have an amino acid sequence that contains one or more insertions, deletions, substitutions or additions to the amino acid sequence of the truncated DN CEBPB proteins identified in Example 1 and Table 4 (SEQ ID NOs: 3-6, or 65, 67, 69, 71, 73, 75, or 77-80) that retain at least, or at least about, 10%, 20%, 30%, 40%, 50%, 60%, 70%, 80%, or 90% or more of the function of the leucine zipper domain of SEQ ID NO: 1 to bind to the protein binding partners of CEBPB. In some implementations, the variant of truncated DN CEBPB protein has a CEBPB leucine zipper domain having at least, or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology or sequence identity to the amino acid sequence of SEQ ID NOs: 3-6, 65, 67, 69, 71, 73, 75, or 77-80.

As described herein, the DN CEBPD protein consists essentially of a CEBPD leucine zipper domain capable of binding to binding partners of CEBPD. For example, in various implementations, the CEBPD leucine zipper domain can have an amino acid sequence KLVELSAENEKLHQRVEQLTRDLAGLRQFFK (SEQ ID NO: 2).

The parent CEBPD leucine zipper domain of SEQ ID NO: 2 includes at least the heptad repeats shown in Table 2. Using the nomenclature described above, each of the listed heptad repeats begins with a residue in the g position followed sequentially by residues in the a through f positions.

TABLE 2
Amino acid sequences of DN CEBPD constructs

| Construct | Amino acid sequence |
|---|---|
| D-1 | KLVELSA (SEQ ID NO: 7) |
| D-2 | ENEKLHQ (SEQ ID NO: 8) |
| D-3 | RVEQLTR (SEQ ID NO: 9) |
| D-4 | DLAGLRQ (SEQ ID NO: 10) |

Truncated DN CEBPD proteins may include one, two, or three, or of the heptad repeats listed in Table 2. Example 1 and Table 3 provide exemplary truncated DN CEBPD proteins.

The present disclosure also contemplates and encompasses variants of truncated DN CEBPD proteins, wherein the leucine zipper domain can have an amino acid sequence that contains one or more insertions, deletions, substitutions or additions to the amino acid sequence of the truncated DN CEBPD proteins identified in Example 1 and Table 3 (SEQ ID NOs.: 7-10, or 50, 52, 54, 56, 58, or 81-106) that retain at least, or at least about, 10%, 20%, 30%, 40%, 50%, 60%, 70%, 80%, or 90% or more of the function of the leucine zipper domain of SEQ ID NO: 2 to bind to the protein binding partners of CEBPD. In some implementations, the variant of truncated DN CEBPD protein has a CEBPD leucine zipper domain having at least, or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology or sequence identity to the amino acid sequence of SEQ ID NOs: 7-10, or 50, 52, 54, 56, 58, or 81-106.

The generation of variant truncated DN CEBPB proteins having variant sequences of CEBPB leucine zipper domains and screening for function of the variant truncated CEBPB leucine zipper domain to bind to the protein binding partners of CEBPB can be performed by skilled persons using methods known in the art. In some implementations, the truncated DN CEBPB proteins having variant sequences of CEBPB leucine zipper domains can be optimized for efficacy and potency by employing the standard techniques of modeling, amino acid substitution, chemical modifications, use of d-amino acids, and the like, and iterative biological testing for activity and stability. It is contemplated that truncated DN CEBPB proteins having variant CEBPB leucine zipper sequences can be provided by processes of protein engineering, such as those employing rational protein design and/or directed evolution. As would be understood by skilled persons, rational design of proteins refers to an approach of creating new proteins or proteins having sequence variation with a certain functionality, based upon the ability to predict how the molecule's structure will affect its behavior through physical models. This can be done either de novo or by making calculated variations on a known structure. In general, site-directed mutagenesis methods identifiable by skilled persons can be used to introduce one or more insertions, deletions, substitutions or additions to the amino acid sequence. In contrast, in directed evolution approaches, random mutagenesis, e.g. by error-prone PCR or Sequence Saturation Mutagenesis, is applied to a protein, and a selection regime is used to select variants having desired traits. Further rounds of mutation and selection may then be applied. Generally, directed evolution approached follow an iterative two-step process which involves generation of protein mutant libraries, and high throughput screening processes to select for variants with improved traits. This technique does not require prior knowledge of the protein structure and function relationship. Directed evolution utilizes random or focused mutagenesis to generate libraries of mutant proteins. Random mutations can be introduced using either error prone PCR, or site saturation mutagenesis, among other methods identifiable by skilled persons. Screening of variant truncated DN CEBPB proteins produced by rational design or directed evolution processes having a leucine zipper domain sequence containing one or more insertions, deletions, substitutions or additions to one or more of the amino acid sequence of SEQ ID NOs: 3-6, 65, 67, 69, 71, 73, or 75 can be performed by any method known in the art. For example, phage display methods are one approach for screening proteins. This method involves the fusion of genes encoding the variant polypeptides with phage coat protein genes. Protein variants expressed on phage surfaces are selected by binding with immobilized targets in vitro. Phages with selected protein variants are then amplified in bacteria, followed by the identification of positive clones by enzyme linked immunosorbent assay. These selected phages are then subjected to DNA sequencing. Cell surface display systems can also be utilized to screen mutant polypeptide libraries. The library mutant genes are incorporated into expression vectors which are then transformed into appropriate host cells. These host cells are subjected to further screening methods to identify the cells with desired phenotypes. Other methods may also be used to screen binding activity of variants of truncated DN CEBPB proteins, such as pull-down assays. For example, polynucleotide expression constructs encoding variant truncated DN CEBPB proteins having a leucine zipper domain sequence containing one or more insertions, deletions, substitutions or additions to one or more of the amino acid sequence of SEQ ID NOs: 3-6, 65, 67, 69, 71, 73, or 75 can be produced having a protein tag, such as a GFP tag or a FLAG tag, among others, fused to the variant truncated DN CEBPB protein. Cultured cells can then be transfected with the expression construct, and protein lysates purified from the cells expressing the construct. The tagged truncated DN CEBPB fusion protein can then be pulled down using antibodies against the tag, and Western blots used to assess the presence of binding partners bound to the variant truncated DN CEBPB protein using antibodies against known binding partners of CEBPB. The present disclosure also contemplates the generation of variant truncated DN CEBPB proteins having variant sequences of CEBPB leucine zipper domains by synthesis of polypeptides in vitro, e.g., by chemical means or in vitro translation of mRNA, and screening thereof for function of the variant CEBPB leucine zipper domain to bind to the protein binding partners of CEBPB. For example, variant truncated DN CEBPB proteins may be synthesized by methods commonly known to one skilled in the art (Modern Techniques of Peptide and Amino Acid Analysis (New York: John Wiley & Sons, 1981); Bodansky, M., Principles of Peptide Synthesis (New York: Springer-Verlag New York, Inc., 1984). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. The amino acid sequences of the present disclosure may contain coupling agents and protecting groups, which are used in the synthesis of protein sequences, and which are well known to one of skill in the art. Similar approaches and methods can be used for producing variants of the truncated DN CEBPD having a leucine zipper domain sequence containing one or more insertions, deletions, substitutions or additions to one or more of the amino acid sequence of SEQ ID NOs: 7-10, 50, 52, 54, 56, 58, or 81-106 described herein and screening the activity of the variant proteins.

In addition to the leucine zipper domain, the truncated DN CEBPB and truncated DN CEBPD proteins of the present disclosure may include one or more amino acid residues, provided that the one or more additional amino acid residues do not prevent the function of the CEBPB or CEBPD leucine zipper from binding to the CEBPB, CEBPD, or ATF5 binding partners.

In various implementations, a functional DNA binding domain is absent from the truncated dominant negative CEBPB protein. In some implementations, a functional DNA binding domain may have no more than, or no more than about, 10%, 20%, 30%, 40% or 50% of the DNA binding function of the DNA binding domain of wild-type CEBPB, such as that of NCBI Accession No. NP_001272808.1 (human isoform c) or NCBI Accession No. NP_001272807.1 (human isoform b) or NCBI Accession No. NP_005185.2 (human isoform a).

In various implementations, a functional DNA binding domain is absent from the truncated dominant negative CEBPD protein. In some implementations, a functional DNA binding domain may have no more than 10%, 20%, or 30% of the DNA binding function of the DNA binding domain of wild-type CEBPD, such as that of NCBI Accession No. NP_005186.

The term “DNA binding domain” refers to a protein domain that contains at least one structural motif configured to recognize and bind double- or single-stranded DNA, wherein the term “motif” refers to a supersecondary structure that appears in multiple proteins, and in particular a three-dimensional protein structure of several adjacent elements of a secondary structure that is typically smaller than a protein domain or a subunit. DNA-binding domains can be part of a larger protein consisting of further protein domains with differing functions including the function of regulating the activity of the DNA-binding domain. The function of DNA binding can be either structural or involve transcription regulation, or both. Many proteins involved in the regulation of gene expression contain DNA-binding domains as will be understood by a skilled person. Such proteins include transcription factors, or transcriptional repressors, among others recognizable by a skilled person.

A DNA-binding domain in the sense of the disclosure can recognize and bind DNA in a DNA sequence-specific or non-sequence-specific manner, which involves molecular complementarity between protein and DNA. The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred.

In some implementations, a DNA-binding domain of a protein can perform DNA recognition and DNA specific binding for example at the major or minor groove of DNA, or at the sugar-phosphate DNA backbone. DNA-binding domains can recognize specific DNA sequences, such as some DNA-binding domains of transcription factors that activate specific genes, or some DNA-binding domains of transcriptional repressors that repress the transcription of specific genes. Another example is that of enzymes that modify DNA at specific sites, such as restriction enzymes. In particular, the DNA binding domain adopts correctly-oriented alignment of its constituent sub-components to effectively interact with DNA.

The specificity of DNA-binding proteins can be detected using many biochemical and biophysical techniques, such as gel electrophoresis, analytical ultracentrifugation, calorimetry, DNA mutation, protein structure mutation or modification, nuclear magnetic resonance, x-ray crystallography, surface plasmon resonance, electron paramagnetic resonance, cross-linking and microscale thermophoresis (MST), among others recognizable by a skilled person. Other assays of DNA binding domain function can include assays of cell viability or function, such as gene expression profiling cell death assays, apoptosis assays, among others, to detect a cell viability or function that is associated with function of a DNA binding domain. Accordingly, for example, the cellular effects of a mutation of a DNA binding domain in a protein may be assessed by using assays of cell viability or function.

In implementations herein described where the truncated DN CEBPB or truncated DN CEBPD lacking a functional DNA binding domain forms a dimer with its binding partner in a cell, the truncated DN CEBPB or truncated DN CEBPD prevents normal dimerization of DNA binding domains and thereby prevents binding with a DNA regulatory sequence upon dimerization of the protein monomers. The term “dimerization” refers to the process of forming a dimer of two monomers, for example two protein monomers. In particular, dimerization dependent DNA binding domains are configured so that dimerization of the monomer components strengthens the interactions of the domain with a corresponding DNA regulatory sequence, rendering the formation or dissociation of the dimers an intrinsic part of the regulatory mechanisms. In particular, dimerization dependent DNA binding domains can bind to DNA sequences that are composed of two very similar “half-sites,” typically also arranged symmetrically. This arrangement allows each protein monomer to make a nearly identical set of contacts and enormously increases the binding affinity.

In some implementations, the dimerization dependent DNA binding domains are leucine zipper domains. In other implementations, dimerization dependent DNA binding domains may be selected from helix-loop-helix, helix-turn-helix, zinc finger, winged helix, winged helix turn helix, helix loop helix, HMG-box, Wor3 domain, OB-fold domain, immunoglobulin fold, B3 domain, TAL effector DNA-binding domain, and others recognizable by a skilled person.

In particular, in some implementations, a functional DNA binding domain of a CEBPB protein may have an amino acid sequence KKTVDKHSDEYKIRRERNNIAVRKSRDKAKMRN (SEQ ID NO: 11). In some implementations, substitution of the bold underlined amino acids of SEQ ID NO: 11 from basic to acidic amino acids can be performed to disrupt binding to DNA. Accordingly, in some implementations, one or more of the bold underlined amino acids of SEQ ID NO: 11 can be mutated to produce a non-functional CEBPB DNA binding domain in a truncated DN CEBPB protein. In some implementations, the specificity of a mutated DNA binding domain of a CEBPB protein can be assessed using biochemical and biophysical techniques, and the cellular effects of a mutated DNA binding domain of a CEBPB protein may be assessed by using assays of cell viability or function, as described herein.

In some implementations, a functional DNA binding domain of a CEBPD protein may have an amino acid sequence DRGSPEYRQRRERNNIAVRKSRDKAKRRNQEMQQK (SEQ ID NO: 12). In some implementations, substitution of the bold underlined amino acids of SEQ ID NO: 12 from basic to acidic amino acids can be performed to disrupt binding to DNA. Accordingly, in some implementations, one or more of the bold underlined amino acids of SEQ ID NO: 12 can be mutated to produce a non-functional CEBPD DNA binding domain in a truncated DN CEBPD protein. In some implementations, the specificity of a mutated DNA binding domain of a CEBPD protein can be assessed using biochemical and biophysical techniques, and the cellular effects of a mutated DNA binding domain of a CEBPD protein may be assessed by using assays of cell viability or function, as described herein.

In some implementations, the truncated DN CEBPB or truncated DN CEBPD protein may include a protein tag. For example, in the truncated DN proteins may contain a MYC tag having three consecutive repetitions of the MYC sequence EQKLISEEDL (SEQ ID NO: 13), EQKLISEEDLEQKLISEEDLEQKLISEEDL (SEQ ID NO: 14) following the N-terminal methionine. As would be understood by skilled persons, a tag is not required for the DN function, but allows additional, optional functionality, for example such as allowing immunoprecipitation or isolation on beads coated with MYC antibodies and can function as a reporter for detecting expression of the DN construct in Western blots or in cells by immunostaining.

The term “tag” as used herein means protein tags including peptide sequences typically introduced onto a recombinant protein. Tags can be removable by chemical agents or by enzymatic means, such as proteolysis or splicing. Tags can be attached to proteins for various purposes: Affinity tags are appended to proteins so that they can be purified from their crude biological source using an affinity technique. These include chitin binding protein (CBP), and the poly(His) tag. The poly(His) tag is a widely-used protein tag; it binds to metal matrices. Chromatography tags can be used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include V5-tag, Myc-tag, HA-tag and NE-tag. These tags are particularly useful for western blotting, immunofluorescence and immunoprecipitation experiments, although they also find use in antibody purification. Protein tags can allow specific enzymatic modification (such as biotinylation by biotin ligase) or chemical modification (such as reaction with FLASH-EDT2 for fluorescence imaging). Tags can be combined, in order to connect proteins to multiple other components. However, with the addition of each tag comes the risk that the native function of the protein may be abolished or compromised by interactions with the tag. Therefore, after purification, tags are sometimes removed by specific proteolysis (e.g. by TEV protease, Thrombin, Factor Xa or Enteropeptidase).

Exemplary tags include without limitation the following, among others known to persons skilled in the art: Peptide tags, such as: AviTag, a peptide allowing biotinylation by the enzyme BirA and so the protein can be isolated by streptavidin (GLNDIFEAQKIEWHE (SEQ ID NO: 15)); Calmodulin-tag, a peptide that can be bound by the protein calmodulin (KRRWKKNFIAVSAANRFKKISSSGAL (SEQ ID NO: 16)); polyglutamate tag, a peptide binding efficiently to anion-exchange resin such as Mono-Q (EEEEEE (SEQ ID NO: 17)); E-tag, a peptide recognized by an antibody (GAPVPYPDPLEPR (SEQ ID NO: 18)); FLAG-tag, a peptide recognized by an antibody (DYKDDDDK (SEQ ID NO: 19)); HA-tag, a peptide from hemagglutinin recognized by an antibody (YPYDVPDYA (SEQ ID NO: 20)); His-tag, typically 5-10 histidines that can be bound by a nickel or cobalt chelate (HHHHHH (SEQ ID NO: 21), HHHHHHHHHH (SEQ ID NO: 22); Myc-tag, a peptide derived from c-myc recognized by an antibody (EQKLISEEDL (SEQ ID NO: 13)); NE-tag, a novel 18-amino-acid synthetic peptide (TKENPRSNQEESYDDNES (SEQ ID NO: 23)) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, a peptide derived from Ribonuclease A (KETAAAKFERQHMDS (SEQ ID NO: 24)); SBP-tag, a peptide which binds to streptavidin (MDEKTTGWRGGHVVEGLAGELEQLRARLEHHPQGQREP (SEQ ID NO: 25)); Softag 1, for mammalian expression (SLAELLNAGLGGS (SEQ ID NO: 26)); Softag 3, for prokaryotic expression (TQDPSRVG (SEQ ID NO: 27)); Strep-tag, a peptide which binds to streptavidin or the modified streptavidin called streptactin (Strep-tag II: WSHPQFEK (SEQ ID NO: 28)); TC tag, a tetracysteine tag that is recognized by FLASH and ReAsH biarsenical compounds (CCPGCC (SEQ ID NO: 29)); V5 tag, a peptide recognized by an antibody (GKPIPNPLLGLDST (SEQ ID NO: 30)); VSV-tag, a peptide recognized by an antibody (YTDIEMNRLGK (SEQ ID NO: 31)); Xpress tag (DLYDDDDK (SEQ ID NO: 32)); Covalent peptide tags such as: Isopeptag, a peptide which binds covalently to pilin-C protein (TDKDMTITFTNKKDAE (SEQ ID NO: 33)); SpyTag, a peptide which binds covalently to SpyCatcher protein (AHIVMVDAYKPTK (SEQ ID NO: 34)); SnoopTag, a peptide which binds covalently to SnoopCatcher protein (KLGDIEFIKVNK (SEQ ID NO: 35)). In implementations of DN proteins described herein, any of the tags described herein, and other tags known to those skilled in the art, can include one or more amino acid substitutions, insertions, or deletions that do not alter the function of the tag, and can further include one or more additional amino acids, up to a maximum tag length of 100 amino acids.

In some implementations, the truncated dominant negative CEBPB or CEBPD protein may have a cell penetrating peptide linked directly or indirectly to the leucine zipper domain.

As used herein, a “cell-penetrating protein”, “cell-penetrating peptide” or “CP” is a peptide that has a short amino acid sequence (e.g., in certain implementations, about 12-30 residues) or functional motif that confers the energy-independent or non-endocytotic translocation properties associated with transport of the membrane-permeable complex across the plasma and/or nuclear membranes of a cell. Representative amino acid motifs conferring such properties are listed in U.S. Pat. No. 6,348,185, the contents of which are expressly incorporated herein by reference. The cell-penetrating peptides described herein preferably include, but are not limited to, penetratin 1 (also referred to herein as “penetratin” or “pen”), transportan, pIs1, TAT (48-60), pVEC, MTS, and MAP.

The cell-penetrating peptides described herein may include without limitation those sequences that retain certain structural and functional features of the identified cell-penetrating peptides, yet differ from the identified peptides' amino acid sequences at one or more positions. Such polypeptide variants can be prepared by substituting, deleting, or adding amino acid residues from the original sequences via methods known in the art.

In some implementations, such substantially similar sequences include sequences that incorporate conservative amino acid substitutions, as described above in connection with polypeptide apoptotic target inhibitors. In some implementations, a cell-penetrating peptide of the present disclosure has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of the identified peptide and is capable of mediating cell penetration.

In some implementations of the present disclosure, the cell-penetrating peptide is penetratin 1, including the peptide sequence RQIKIWFQNRRMKWKK (SEQ ID NO: 36), or a conservative variant thereof. As used herein, a “conservative variant” is a peptide having one or more amino acid substitutions, wherein the substitutions do not adversely affect the shape—or, therefore, the biological activity, such as transport activity, or membrane toxicity—of the cell-penetrating peptide.

Penetratin 1 is a 16-amino-acid polypeptide derived from the third alpha-helix of the homeodomain of Drosophila antennapedia. Its structure and function have been well studied and characterized: Derossi et al., Trends Cell Biol., 8 (2): 84-87, 1998; Dunican et al., Biopolymers, 60 (1): 45-60, 2001; Hallbrink et al., Biochim. Biophys. Acta, 1515 (2): 101-09, 2001; Bolton et al., Eur. J. Neurosci., 12 (8): 2847-55, 2000; Kilk et al., Bioconjug. Chem., 12 (6): 911-16, 2001; Bellet-Amalric et al., Biochim. Biophys. Acta, 1467 (1): 131-43, 2000; Fischer et al., J. Pept. Res., 55 (2): 163-72, 2000; Thoren et al., FEBS Lett., 482 (3): 265-68, 2000.

It has been shown that penetratin1 efficiently carries avidin, a 63-kDa protein, into human Bowes melanoma cells (Kilk et al., Bioconjug. Chem., 12 (6): 911-16, 2001). Additionally, it has been shown that the transportation of penetratin1 and its cargo is non-endocytotic and energy-independent, and does not depend upon receptor molecules or transporter molecules. Furthermore, it is known that penetratin1 is able to cross a pure lipid bilayer (Thoren et al., FEBS Lett., 482 (3): 265-68, 2000). This feature enables penetratin1 to transport its cargo, free from the limitation of cell-surface-receptor/-transporter availability. The delivery vector previously has been shown to enter all cell types (Derossi et al., Trends Cell Biol., 8 (2): 84-87, 1998), and effectively to deliver peptides (Troy et al., Proc. Natl. Acad. Sci. USA, 93:5635-40, 1996) or antisense oligonucleotides (Troy et al., J. Neurosci., 16:253-61, 1996; Troy et al., J. Neurosci., 17:1911-18, 1997).

Other non-limiting implementations of the present disclosure involve the use of the following exemplary cell permeant molecules: RL16 (RRLRRLLRRLLRRLRR (SEQ ID NO: 37)), a sequence derived from Penetratin1 with slightly different physical properties (Biochim Biophys Acta. 2008 July-August; 1780 (7-8): 948-59); and RVGRRRRRRRRR (SEQ ID NO: 38), a rabies virus sequence which targets neurons see P. Kumar, H. Wu, J. L. McBride, K. E. Jung, M. H. Kim, B. L. Davidson, S. K. Lec, P. Shankar and N. Manjunath, Transvascular delivery of small interfering RNA to the central nervous system, Nature 448 (2007), pp. 39-43.

In some non-limiting implementations of the present disclosure, the cell-penetrating peptide can be a cell-penetrating peptide selected from the group consisting of: transportan, pIS1, Tat (48-60), pVEC, MAP, and MTS. Transportan is a 27-amino-acid long peptide containing 12 functional amino acids from the amino terminus of the neuropeptide galanin, and the 14-residue sequence of mastoparan in the carboxyl terminus, connected by a lysine (Pooga et al., FASEB J., 12 (1): 67-77, 1998). It includes the amino acid sequence GWTLNSAGYLLGKINLKALAALAKKIL (SEQ ID NO: 39), or a conservative variant thereof.

pIs1 is derived from the third helix of the homeodomain of the rat insulin 1 gene enhancer protein (Magzoub et al., Biochim. Biophys. Acta, 1512 (1): 77-89, 2001; Kilk et al., Bioconjug. Chem., 12 (6): 911-16, 2001). pIs1 includes the amino acid sequence PVIRVW FQNKRCKDKK (SEQ ID NO: 40), or a conservative variant thereof.

Tat is a transcription activating factor, of 86-102 amino acids, that allows translocation across the plasma membrane of an HIV-infected cell, to transactivate the viral genome (Hallbrink et al., Biochem. Biophys. Acta., 1515 (2): 101-09, 2001; Suzuki et al., J. Biol. Chem., 277 (4): 2437-43, 2002; Futaki et al., J. Biol. Chem., 276 (8): 5836-40, 2001). A small Tat fragment, extending from residues 48-60, has been determined to be responsible for nuclear import (Vives et al., J. Biol. Chem., 272 (25): 16010-017, 1997); it includes the amino acid sequence: YGRKKRRQRRR (SEQ ID NO: 41); GRKKRRQRRRPPQ (SEQ ID NO: 42); or a conservative variant thereof.

pVEC is an 18-amino-acid-long peptide derived from the murine sequence of the cell-adhesion molecule, vascular endothelial cadherin, extending from amino acid 615-632 (Elmquist et al., Exp. Cell Res., 269 (2): 237-44, 2001). pVEC includes the amino acid sequence LLIILRRRIRKQAHAH (SEQ ID NO: 43), or a conservative variant thereof.

MTSs, or membrane translocating sequences, are those portions of certain peptides which are recognized by the acceptor proteins that are responsible for directing nascent translation products into the appropriate cellular organelles for further processing (Lindgren et al., Trends in Pharmacological Sciences, 21 (3): 99-103, 2000; Brodsky, J. L., Int. Rev. Cyt., 178:277-328, 1998; Zhao et al., J. Immunol. Methods, 254 (1-2): 137-45, 2001). An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides (Lindgren et al., Trends in Pharmacological Sciences, 21 (3): 99-103, 2000; Morris et al., Nucleic Acids Res., 25:2730-36, 1997). MPS includes the amino acid sequence GALFLGWLGAAGSTMGAWSQPKKKRKV (SEQ ID NO: 44), or a conservative variant thereof.

Model amphipathic peptides, or MAPs, form a group of peptides that have, as their essential features, helical amphipathicity and a length of at least four complete helical turns (Scheller et al., J. Peptide Science, 5 (4): 185-94, 1999; Hallbrink et al., Biochim. Biophys. Acta., 1515 (2): 101-09, 2001). An exemplary MAP includes the amino acid sequence KLALKLALKALKAALKLA (SEQ ID NO: 45), or a conservative variant thereof.

In some implementations, the cell-penetrating peptides described herein can be covalently bound to another protein, such as the truncated DN-CEBPB or truncated DN-CEBPD proteins described herein, e.g., via a peptide bond. In some implementations, the cell-penetrating peptide is operably linked to another protein, such as the truncated DN proteins described herein via recombinant DNA technology. For example, the truncated DN proteins described herein can be introduced either upstream (for linkage to the amino terminus of the cell-penetrating peptide) or downstream (for linkage to the carboxy terminus of the cell-penetrating peptide), or both, of a nucleic acid sequence encoding the cell-penetrating peptide of interest. Such fusion sequences including both the truncated DN proteins described herein encoding nucleic acid sequence and the cell-penetrating peptide encoding nucleic acid sequence can be expressed using techniques well known in the art.

In some implementations, the truncated DN-CEBPB or truncated DN-CEBPD proteins described herein can be operably linked to the cell-penetrating peptide via a non-covalent linkage. In some implementations, such non-covalent linkage is mediated by ionic interactions, hydrophobic interactions, hydrogen bonds, or van der Waals forces.

In some implementations, the truncated DN-CEBPB or truncated DN-CEBPD proteins described herein is operably linked to the cell penetrating peptide via a chemical linker. Examples of such linkages typically incorporate 1-30 nonhydrogen atoms selected from the group consisting of C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In some implementations, the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. In some implementations, the linker is a combination of single carbon-carbon bonds and carboxamide, sulfonamide or thioether bonds.

A general strategy for conjugation involves preparing the cell-penetrating peptide and the truncated DN components separately, wherein each is modified or derivatized with appropriate reactive groups to allow for linkage between the two. The modified truncated DN component is then incubated together with a cell-penetrating peptide that is prepared for linkage, for a sufficient time (and under such appropriate conditions of temperature, pH, molar ratio, etc.) as to generate a covalent bond between the cell-penetrating peptide and the truncated DN component.

The present disclosure contemplates the use of proteins and protein analogues generated by synthesis of polypeptides in vitro, e.g., by chemical means or in vitro translation of mRNA. For example, truncated DN-CEBPB or truncated DN-CEBPD and inhibitors thereof may be synthesized by methods commonly known to one skilled in the art (Modern Techniques of Peptide and Amino Acid Analysis (New York: John Wiley & Sons, 1981); Bodansky, M., Principles of Peptide Synthesis (New York: Springer-Verlag New York, Inc., 1984). Examples of methods that may be employed in the synthesis of the amino acid sequences, and analogues of these sequences, include, but are not limited to, solid-phase peptide synthesis, solution-method peptide synthesis, and synthesis using any of the commercially-available peptide synthesizers. The amino acid sequences of the present disclosure may contain coupling agents and protecting groups, which are used in the synthesis of protein sequences, and which are well known to one of skill in the art.

As used herein, “amino acid residue,” “amino acid,” or “residue,” includes genetically encoded amino acid residues and non-genetically encoded amino acid residues, e.g., non-genetically encoded amino acid residues or non-natural amino acids include, but are not limited to D-enantiomers of naturally occurring chiral amino acids, β-alanine (β-Ala); 2,3-diaminopropionic acid (Dpr); nipecotic acid (Nip); pipecolic acid (Pip); ornithine (Orn); citrulline (Cit); t-butylalanine (t-BuA); 2-t-butylglycine (t-BuG); N-methylisoleucine (Melle); phenylglycine (PhG); cyclohexylalanine (ChA); norleucine (Nle); naphthylalanine (Nal); 4-chlorophenylalanine (Phe(4-C1)); 2-fluorophenyl alanine (Phe(2-F)); 3-fluorophenyl alanine (Phe(3-F)); 4-fluorophenylalanine (Phe(4-F)); penicillamine (Pen); 1,2,3,4-tetrahydroisoquinoline-3-carboxylic acid (Tic); B-2-thienylalanine (Thi); methionine sulfoxide (MSO); homoarginine (hArg); N-acetyl lysine (AcLys); 2,4-diaminobutyric acid (Dbu); 2,3-diaminobutyric acid (Dab); p-aminophenylalanine (Phe (pNH2)); N-methyl valine (MeVal); homocysteine (hCys), homophenylalanine (hPhe); homoserine (hSer); hydroxyproline (Hyp); homoproline (hPro); and the corresponding D-enantiomer of each of the foregoing, e.g., D-β-Ala, D-Dpr, D-Nip, D-Orn, D-Cit, D-t-BuA, D-t-BuG, D-Melle, D-PhG, D-ChA, D-Nle, D-Nal, D-Phe(4-C1), D-Phe(2-F), D-Phe(3-F), D-Phe(4-F), D-Pen, D-Tic, D-Thi, D-MSO, D-hArg, D-AcLys, D-Dbu, D-Dab, D-Phe(pNH2), D-MeVal, D-hCys, D-hPhe, D-hSer, D-Hyp, and D-hPro. Additional non-genetically encoded amino acid residues include 3-aminopropionic acid; 4-aminobutyric acid; isonipecotic acid (Inp); aza-pipecolic acid (azPip); aza-proline (azPro); α-aminoisobutyric acid (Aib); ε-aminohexanoic acid (Aha); δ-aminovaleric acid (Ava); N-methylglycine (MeGly).

In some implementations of the DN proteins described herein, the penetratin can have an amino acid sequence RQIKIFFQNRRMKFKK (SEQ ID NO: 46) or RQIKIWFRKWKK (SEQ ID NO: 47) (Letoha et al. (2003) Journal of Molecular Recognition 16 (5): 272-279).

In some implementations, a composition is described. The composition includes a truncated dominant negative CEBPB protein described herein, or a truncated dominant negative CEBPD protein described herein, or a combination thereof, and a pharmaceutically acceptable excipient.

For oral administration, the truncated DN-CEBPB, truncated DN-CEBPD, truncated CP-DN-CEBPB, and/or truncated CP-DN-CEBPD composition can be formulated as capsules, tablets, powders, granules, or as a suspension. The composition formulation may have conventional additives, such as lactose, mannitol, corn starch, or potato starch. The composition formulation also may be presented with binders, such as crystalline cellulose, cellulose derivatives, acacia, corn starch, or gelatins. Additionally, the formulation may be presented with disintegrators, such as corn starch, potato starch, or sodium carboxymethylcellulose. The composition formulation also may be presented with dibasic calcium phosphate anhydrous or sodium starch glycolate. Finally, the composition formulation may be presented with lubricants, such as talc or magnesium stearate.

In some implementations, the composition may include, without limitation, a carrier protein, which may increase activity of the truncated DN composition. Without limitation to theory, such a carrier protein may, for example, increase activity by increasing solubility and/or promoting improved protein folding of the truncated DN construct in the composition.

For example, and not by way of limitation, the carrier protein may be a serum albumin, such as bovine serum albumin (BSA) or human serum albumin, among others. For example, human serum albumin is often included in some vaccines and is often given at, or at about, 20% weight/volume (w/v) in infusions for some medical conditions. In some implementations, the serum albumin may be added at a concentration (w/v) of, or of about 5%, 10%, 15%, 20%, 25% or 30%. For example, in some implementations, a composition including, without limitation, a truncated dominant negative protein described herein and a serum albumin, may have increased efficacy and/or potency compared to a composition that does not have a serum albumin. In some implementations, the serum albumin may have a concentration in the composition of 3 mg/ml.

In some implementations, the composition may include, without limitation, a mixture of glutamate and arginine. Without limitation to theory, glutamine and arginine may, for example, increase activity of the truncated DN composition by increasing solubility and/or stability of the truncated DN construct in the composition. In some implementations, the mixture of arg/glu may be added at a molarity of, or of about 5 mM, 10 mM, 15 mM, 20 mM, 25 mM, or 30 mM.

In some implementations, the increased efficacy and/or potency resulting from including the carrier and/or other additive may be additive. In some implementations, the increased efficacy and/or potency may be synergistic.

For parenteral administration, administration by injection through a route other than the alimentary canal, the truncated DN composition can be combined with a sterile aqueous solution that is preferably isotonic with the blood of the subject. Such a truncated DN composition formulation can be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The truncated DN composition formulation can be presented in unit or multi-dose containers, such as sealed ampoules or vials. The truncated DN composition formulation can be delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual.

In some implementations, the truncated DN composition formulation is prepared for intranasal delivery. For nasal administration, solutions or suspensions including the truncated DN composition formulation can be prepared for direct application to the nasal cavity by conventional means, for example with a dropper, pipette or spray. Other means for delivering the nasal spray composition, such as inhalation via a metered dose inhaler (MDI), may also be used according to the present disclosure. Several types of MDIs are regularly used for administration by inhalation. These types of devices can include breath-actuated MDI, dry powder inhaler (DPI), spacer/holding chambers in combination with MDI, and nebulizers. The term “MDI” as used herein refers to an inhalation delivery system including, for example, a canister containing an active agent dissolved or suspended in a propellant optionally with one or more excipients, a metered dose valve, an actuator, and a mouthpiece. The canister is usually filled with a solution or suspension of an active agent, such as the nasal spray composition, and a propellant, such as one or more hydrofluoroalkanes. When the actuator is depressed a metered dose of the solution is aerosolized for inhalation. Particles including the active agent are propelled toward the mouthpiece where they may then be inhaled by a subject. The formulations may be provided in single or multidose form. For example, in the case of a dropper or pipette, this may be achieved by the patient administering an appropriate, predetermined volume of the solution or suspension. In the case of a spray, this may be achieved for example by means of a metering atomizing spray pump. To improve nasal delivery and retention the components according to the disclosure may be encapsulated with cyclodextrins, or formulated with agents expected to enhance delivery and retention in the nasal mucosa.

Commercially available administration devices that are used or can be adapted for nasal administration of a composition of the disclosure include the AERONEB™ (Aerogen, San Francisco, Calif.), AERONEB GO™ (Aerogen); PARI LC PLUS™, PARI BOY™ N, PARI™ eflow (a nebulizer disclosed in U.S. Pat. No. 6,962,151), PARI LC SINUS™, PARI SINUSTAR™, PARI SINUNEB™, VibrENT™ and PARI DURANEB™ (PART Respiratory Equipment, Inc., Monterey, Calif. or Munich, Germany); MICROAIR™ (Omron Healthcare, Inc, Vernon Hills, Ill.), HALOLITE™ (Profile Therapeutics Inc, Boston, Mass.), RESPIMAT™ (Boehringer Ingelheim, Germany), AERODOSE™ (Aerogen, Inc, Mountain View, Calif.), OMRON ELITE™ (Omron Healthcare, Inc, Vernon Hills, Ill.), OMRON MICROAIR™ (Omron Healthcare, Inc, Vernon Hills, MABISMIST™ II (Mabis Healthcare, Inc, Lake Forest, Ill.), LUMISCOPE™ 6610, (The Lumiscope Company, Inc, East Brunswick, N.J.), AIRSEP MYSTIQUE™, (AirSep Corporation, Buffalo, N.Y.), ACORN-1™ and ACORN-II™ (Vital Signs, Inc, Totowa, N.J.), AQUATOWER™ (Medical Industries America, Adel, Iowa), AVA-NEB™ (Hudson Respiratory Care Incorporated, Temecula, Calif.), AEROCURRENT™ utilizing the AEROCELL™ disposable cartridge (AerovectRx Corporation, Atlanta, Ga.), CIRRUS™ (Intersurgical Incorporated, Liverpool, N.Y.), DART™ (Professional Medical Products, Greenwood, S.C.), DEVILBISS™ PULMO AIDE (DeVilbiss Corp; Somerset, Pa.), DOWNDRAFT™ (Marquest, Englewood, Colo.), FAN JET™ (Marquest, Englewood, Colo.), MB-5™ (Mefar, Bovezzo, Italy), MISTY NEB™ (Baxter, Valencia, Calif.), SALTER 8900™ (Salter Labs, Arvin, Calif.), SIDESTREAM™ (Medic-Aid, Sussex, UK), UPDRAFT-II™ (Hudson Respiratory Care; Temecula, Calif.), WHISPER JET™ (Marquest Medical Products, Englewood, Colo.), AIOLOS™ (Aiolos Medicinsk Teknik, Karlstad, Sweden), INSPIRON™ (Intertech Resources, Inc., Bannockburn, Ill.), OPTIMIST™ (Unomedical Inc., McAllen, Tex.), PRODOMO™, SPIRA™ (Respiratory Care Center, Hameenlinna, Finland), AERx™ Essence™ and Ultra™, (Aradigm Corporation, Hayward, Calif.), SONIK™ LDI Nebulizer (Evit Labs, Sacramento, Calif.), ACCUSPRAY™ (BD Medical, Franklin Lake, N.J.), ViaNase ID™ (electronic atomizer; Kurve, Bothell, Wash.), OptiMist™ device or OPTINOSE™ (Oslo, Norway), MAD Nasal™ (Wolfe Tory Medical, Inc., Salt Lake City, Utah), Freepod™ (Valois, Marly le Roi, France), Dolphin™ (Valois), Monopowder™ (Valois), Equadel™ (Valois), VP3™ and VP7™ (Valois), VP6 Pump™ (Valois), Standard Systems Pumps™ (Ing. Erich Pfeiffer, Radolfzell, Germany), AmPump™ (Ing. Erich Pfeiffer), Counting Pump™ (Ing. Erich Pfeiffer), Advanced Preservative Free System™ (Ing. Erich Pfeiffer), Unit Dose System™ (Ing. Erich Pfeiffer), Bidose System™ (Ing. Erich Pfeiffer), Bidose Powder System™ (Ing. Erich Pfeiffer), Sinus Science™ (Aerosol Science Laboratories, Inc., Camarillo, Calif.), ChiSys™ (Archimedes, Reading, UK), Fit-Lizer™ (Bioactis, Ltd, a SNBL subsidiary (Tokyo, J P), Swordfish V™ (Mystic Pharmaceuticals, Austin, Tex.), DirectHaler™ Nasal (DirectHaler, Copenhagen, Denmark) and SWIRLER™ Radioaerosol System (AMICI, Inc., Spring City, Pa.).

For transdermal administration, truncated DN-CEBPB, truncated DN-CEBPD, truncated CP-DN-CEBPB, and/or truncated CP-DN-CEBPD can be combined with skin penetration enhancers, such as propylene glycol, polyethylene glycol, isopropanol, ethanol, oleic acid, N-methylpyrrolidone, and the like, which increase the permeability of the skin to the truncated DN construct, and permit the truncated DN construct to penetrate through the skin and into the bloodstream. The truncated DN composition also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch.

The pharmaceutically-acceptable carrier should be “acceptable” in the sense of being compatible with the other ingredients of the composition, and ideally not deleterious to the recipient thereof, or having acceptable tolerability in the subject. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The truncated DN composition formulations described herein can be prepared by methods well-known in the pharmaceutical arts. For example, the truncated DN-CEBPB, truncated DN-CEBPD, truncated CP-DN-CEBPB, and/or truncated CP-DN-CEBPD can be brought into association with a carrier or diluent, as a suspension or solution. Optionally, one or more accessory ingredients (e.g., buffers, flavoring agents, surface active agents, and the like) also can be added. The choice of carrier will depend upon the route of administration. The pharmaceutical composition would be useful for administering the truncated DN construct to a subject to treat a tumor and/or neoplastic cell, as discussed herein. The truncated DN construct is provided in an amount that is effective to treat the tumor and/or neoplastic cell in a subject to whom the pharmaceutical composition is administered. That amount may be readily determined by the skilled artisan, as described herein.

Compositions of the present disclosure can further include other therapeutic agents. For example, they can include any one or more anti-cancer agents. In some implementations, the one or more anti-cancer agent will be selected from the group consisting of: alkylating agents; anti-metabolites; anti-microtubule agents; topoisomerase inhibitors, antibiotics, and antibodies/antibody-drug conjugates. The amounts of those anti-cancer agents in compositions of the present disclosure can, in some implementations, be reduced as compared to normal doses of such agents administered in a similar fashion.

Compositions of the present disclosure can further be administered in combination with other therapeutic agents such as inhibitors of growth factor receptor signaling, of proteasome activity, of oncogenic kinases and of other oncogenic proteins, STAT3 inhibitors, and/or BH3-mimetics, among others identifiable by skilled persons.

Compositions of the present disclosure can further be administered in combination with other cancer treatments, such as radiation treatment, immunotherapeutics, anti-microtubule agents, alkylating agents, hypomethylating agents, and/or anti-metabolites, among others.

In some implementations, a method of decreasing activity or viability of a neoplastic cell is described. The method includes contacting the neoplastic cell with a truncated dominant negative CEBPB protein described herein, or a truncated dominant negative CEBPD protein described herein, or cell-penetrating forms thereof, or a combination thereof for a time and under conditions sufficient to cause a decrease in activity or viability of the neoplastic cell.

The term “neoplastic cell”, “neoplasia”, and related terms as used herein, refers to the uncontrolled and progressive multiplication of tumor cells under conditions that would not elicit, or would cause cessation of, multiplication of normal cells. Neoplasia results in the formation of a “neoplasm”, which may refer to any new and abnormal growth, particularly a new growth of tissue, in which the growth of cells is uncontrolled and progressive. As used herein, neoplasms include, without limitation, morphological irregularities in cells in tissue of a subject, as well as pathologic proliferation of cells in tissue of a subject, as compared with normal proliferation in the same type of tissue. Additionally, neoplasms include benign tumors and malignant tumors. Malignant neoplasms are distinguished from benign in that the former show a greater degree of anaplasia, or loss of differentiation and orientation of cells, and have the properties of invasion and metastasis. Thus, neoplasia includes “cancer”, which refers to a proliferation of tumor cells having the unique trait of loss of normal controls, resulting in unregulated growth, lack of differentiation, local tissue invasion, and metastasis.

In some implementations, the neoplastic cell can be selected from the group consisting of: breast, ovary, endometrium, gastric, colon, liver, pancreas, kidney, bladder, prostate, testis, skin, esophagus, tongue, mouth, parotid, larynx, pharynx, lymph node, lung, and brain. In some implementations, the neoplastic cell can be selected from the group consisting of glioblastoma, astrocytoma, glioma, medulloblastoma and neuroblastoma. The neoplastic cells can include solid tumors or hematological cancers.

Additionally, as used herein, the term “neural tumor” refers to a tumorigenic form of neural cells, and includes astrocytoma cells, including, without limitation, Grades I-IV astrocytomas, anaplastic astrocytoma, astroblastoma, astrocytoma fibrillare, astrocytoma protoplasmaticum, gemistocytic astrocytoma, and glioblastoma multiforme), gliomas, medulloblastomas, neuroblastomas, and other brain tumors. Brain tumors invade and destroy normal tissue, producing such effects as impaired sensorimotor and cognitive function, increased intracranial pressure, cerebral edema, and compression of brain tissue, cranial nerves, and cerebral vessels. Metastases may involve the skull or any intracranial structure. The size, location, rate of growth, and histologic grade of malignancy determine the seriousness of brain tumors. Nonmalignant tumors grow slowly, with few mitoses, no necrosis, and no vascular proliferation. Malignant tumors grow more rapidly, and invade other tissues. However, they rarely spread beyond the CNS, because they cause death by local growth.

Brain tumors may be classified by site (e.g., brain stem, cerebellum, cerebrum, cranial nerves, ependyma, meninges, neuroglia, pineal region, pituitary gland, and skull) or by histologic type (e.g., meningioma, primary CNS lymphoma, or astrocytoma). Common primary childhood tumors are cerebellar astrocytomas and medulloblastomas, ependymomas, gliomas of the brain stem, neuroblastomas, and congenital tumors. In adults, primary tumors include meningiomas, schwannomas, and gliomas of the cerebral hemispheres (particularly the malignant glioblastoma multiforme and anaplastic astrocytoma, and the more benign astrocytoma and oligodendroglioma). Overall incidence of intracranial neoplasms is essentially equal in males and females, but cerebellar medulloblastoma and glioblastoma multiforme are more common in males.

Gliomas are tumors composed of tissue representing neuroglia in any one of its stages of development. They account for 45% of intracranial tumors. Gliomas can encompass all of the primary intrinsic neoplasms of the brain and spinal cord, including astrocytomas, ependymomas, and neurocytomas. Astrocytomas are tumors composed of transformed astrocytes, or astrocytic tumor cells. Such tumors have been classified in order of increasing malignancy: Grade I consists of fibrillary or protoplasmic astrocytes; Grade II is an astroblastoma, consisting of cells with abundant cytoplasm and two or three nuclei; and Grades III and IV are forms of glioblastoma multiforme, a rapidly growing tumor that is usually confined to the cerebral hemispheres and composed of a mixture of astrocytes, spongioblasts, astroblasts, and other astrocytic tumor cells. Astrocytoma, a primary CNS tumor, is frequently found in the brain stem, cerebellum, and cerebrum. Anaplastic astrocytoma and glioblastoma multiforme are commonly located in the cerebrum.

For example, but not by way of limitation, neoplastic cells also include cell lines such as U87 (human glioblastoma); U373 (human glioblastoma); LN229 (human glioblastoma); C6 (rat glioblastoma); Me1501 (human melanoma); H2452 (human mesothelioma); MDA-MB-468 (human breast cancer), Panc-1 (human pancreatic cancer); SH-SY5Y (human neuroblastoma cells); HCT-116 (colon-carcinoma cancer); T98G (glioblastoma), MDA-MB-231 (breast cancer), A375 (melanoma), Shep21N (neuroblastoma), and CHLA255 (neuroblastoma).

For any of the above tumors, cancers, or neoplastic cells, peptides as disclosed herein, such as truncated DN-CEBPB, truncated DN-CEBPD, truncated CP-DN-CEBPB, and/or truncated CP-DN-CEBPD, may be administered to a subject in order to inhibit metastasis, to inhibit cancer recurrence from dormant cells, particularly dormant cells not inhibited by other therapeutics due to their dormancy, or to inhibit both metastasis and cancer recurrence from dormant cells. Biopsies, cancer type, and other clinical indicia may be used to identify subject likely to experience metastasis or cancer recurrence from dormant cells.

The term “activity” as it relates to cells includes any cellular function or activity of cells such as neoplastic cells, for example including but limited to growth, intracellular signaling, proliferation, and migration, among others identifiable by skilled persons. The term “viability” generally refers to survival; accordingly, a decrease in viability can include an increase in cell death. In some implementations, contacting a neoplastic cell with a truncated DN CEBPB and/or truncated DN CEBPD, or a cell-penetrating forms thereof described herein may increase apoptosis in the neoplastic cell. In some implementations, contacting a neoplastic cell with a truncated DN construct, or a cell-penetrating forms thereof described herein may decrease growth or proliferation of the neoplastic cell. In some implementations, contacting a neoplastic cell with a truncated DN construct, or a cell-penetrating forms thereof described herein may increase cell death in the neoplastic cell.

In some implementations, the activity and/or viability of a neoplastic cell may be decreased at least 1%, 5%, 10%, 20%, 30%, 40%, 50%, or greater (inclusive of intermediate ranges between those explicitly recited, e.g., 5-10%, 10-20%, 20-30%, 40-50%, or greater than 50% including 50%-100%).

As described herein, CEBPB and CEBPD contain basic leucine zipper motifs that may be used to create a truncated dominant negative that may function as a cancer therapeutic.

Examples 1 and 5 describe exemplary truncated DN constructs including truncated DN-CEBPD, truncated DN-CEBPD, truncated CP-DN-CEBPB, or truncated CP-DN-CEBPB for interference with one or more functions of CEBPB, and/or CEBPD such as signaling including one or more of these proteins, and for treatment of cancers. Examples 2 and 6 provide evidence that exemplary truncated DN constructs can promote apoptotic death of HCT116 colon cancer cell cultures. Examples 3 and 7 provide further evidence that cell penetrating forms of truncated DN constructs repress growth and survival of T98G glioblastoma cells, MDA-MB-231 breast cancer cells, Shep21N neuroblastoma cells and CHLA255 neuroblastoma cells. Example 4 provides further evidence that administration cell penetrating forms of truncated DN constructs to mice with A375 xenograph tumors reduces tumor growth and prolongs animal survival with no effect on animal weight.

Accordingly, in various implementations of the methods described herein and within the scope of the present disclosure, it is expected that truncated DN-forms of CEBPB and/or CEBPD described herein can function as anti-cancer drugs.

In some implementations, a method of treating cancer in a subject is described. The method includes administering to the subject an effective amount of truncated DN-CEBPB, truncated DN-CEBPD, truncated CP-DN-CEBPB, and/or truncated CP-DN-CEBPD.

The methods of the present disclosure can be performed in vitro as well as in vivo in a subject. The term “subject” as used herein in the context of treatment includes a single animal and in particular higher animals and in particular vertebrates such as mammals and in particular human beings.

The dominant negative proteins and compositions thereof described herein can be administered to a subject by any suitable procedure, including, without limitation, oral administration, parenteral administration, intranasal administration, intraperitoneal administration and transdermal administration. In some implementations, the dominant negative proteins and compositions thereof can be administered parenterally, by intracranial, intraspinal, intrathecal, intraperitoneal or subcutaneous injection.

In general, the term “sequence identity” refers to the occurrence of exactly the same nucleotide or amino acid in the same position in aligned sequences. For example, see Pertsemlidis and Fondon III, “Having a BLAST with bioinformatics (and avoiding BLASTphemy)” Genome Biology, 2001 Sep. 27, 2, reviews2002.1. Generally, % amino acid sequence identity values can be readily obtained using, for example, the WU-BLAST-2 computer program (Altschul et al., Methods in Enzymology 266:460-480 (1996)).

As used herein, the term “effective amount” refers to an amount of the proteins or compositions thereof necessary to bring about a desired result, such as but not limited to killing or inhibiting activity of a neoplastic cell.

In addition, the term “effective amount” in some implementations means effective to ameliorate or minimize the clinical impairment or symptoms of the neoplastic cell such as a tumor. For example, the clinical impairment or symptoms of the tumor may be ameliorated or minimized by diminishing any pain or discomfort suffered by the subject; by extending the survival of the subject beyond that which would otherwise be expected in the absence of such treatment; by inhibiting or preventing the development or spread of the tumor; or by limiting, suspending, terminating, or otherwise controlling the maturation and proliferation of cells in the tumor. The amount of truncated DN-CEBPB, truncated DN-CEBPD, truncated CP-DN-CEBPB, and/or truncated CP-DN-CEBPD effective to treat a tumor in a subject in need of treatment will vary depending upon the particular factors of each case, including the type of tumor, the stage of the tumor, the subject's weight, the severity of the subject's condition, and the method of administration. This amount can be readily determined by the skilled artisan.

In some implementations, the truncated DN-CEBPB, truncated DN-CEBPD, truncated CP-DN-CEBPB, and/or truncated CP-DN-CEBPD may be provided in a composition at a concentration of, or of about, 0.1-5.0 mM, or about 5 μM-50 μM, or 10 μM-50 μM.

In some implementations, the truncated DN-CEBPB, truncated DN-CEBPD, truncated CP-DN-CEBPB, and/or truncated CP-DN-CEBPD may be administered to a subject, such as a cancer patient, at a dosage of, or of about, 5-50 mg/kg, about 10-50 mg/mg, or about 10-20 mg/kg.

In accordance with methods described herein, CEBPB, CEBPD, or ATF5, or their cellular binding partners can be inhibited in a neoplastic cell by disabling, disrupting, or inactivating the function or activity of CEBPB, CEBPD, or ATF5 in the neoplastic cell. The function or activity of CEBPB, CEBPD, or ATF5 in the neoplastic cell may be inhibited by contacting the neoplastic cell with a truncated DN-CEBPB, truncated DN-CEBPD, truncated CP-DN-CEBPB, and/or truncated CP-DN-CEBPD capable of inhibiting the function or activity of native CEBPB, CEBPD, or ATF5 in the cell.

In some implementations, function or activity of the CEBPB or CEBPD, in the cell is inhibited by at least 1%, 5%, 10%, 20%, 30%, 40%, 50%, or greater (inclusive of intermediate ranges between those explicitly recited, e.g., 5-10%, 10-20%, 20-30%, 40-50%, or greater than 50% including 50%-100%).

In some implementations, function or activity of the CEBPB or CEBPD is decreased by inhibiting expression of CEBPB or CEBPD. Such expression can be inhibited by at least 1%, 5%, 10%, 20%, 30%, 40%, 50%, or greater (inclusive of intermediate ranges between those explicitly recited, e.g., 5-10%, 10-20%, 20-30%, 40-50%, or greater than 50% including 50%-100%).

In some implementations, the present disclosure relates to a polynucleotide including a sequence encoding a truncated dominant negative CEBPB protein described herein or a truncated dominant negative CEBPD protein described herein. For example, Examples 2 and 6 describe a polynucleotide vector encoding exemplary truncated DN proteins causing expression of the encoded DN proteins in a colon cancer cell line, resulting in cell death. Accordingly, in some implementations, the present disclosure contemplates administering a polynucleotide expression vector encoding the truncated DN proteins described herein to a subject as a therapeutic method. Suitable vectors for delivery of polynucleotides encoding the truncated DN proteins described herein include without limitation recombinant adeno-associated virus (AAV) vectors, among others identifiable by skilled persons.

In still other implementations, peptides as disclosed herein, such as truncated DN-CEBPB, truncated DN-CEBPD, truncated CP-DN-CEBPB, and/or truncated CP-DN-CEBPD, may be administered with other cancer therapeutics to achieve a synergistic effect. Administration may involve co-treatment, pre-treatment, or-post treatment with the peptides as disclosed herein as compared to treatment time with the additional therapeutic. Examples of additional therapeutics include gamma radiation, paclitaxel, chloroquine, 5-Azacytidine (Azacitidine), 5-Aza-2′-deoxycytidine (Decitabine), Guadecitabine, oxorubicin, BH3-mimetics, and hypomethylating agents and chemotherapeutics in the same drug classes or that exert their anti-cancer effects in the same manner as these therapeutics. For example, additional therapeutics may include other taxanes, such as nab-paclitaxel, Abraxane, docetaxel, 10-deacetylbaccatin III, baccatin III, paclitaxel C, and 7-epipaclitaxel. Additional therapeutics may also include other anthracyclines, such as daunorubicin, epirubicin, idarubicin, mitoxantrone, and valrubicin.

Example additional therapeutics further include chemotherapeutics or other therapeutics known to promote apoptosis in cancer cells, such as BH3 mimetics, including ABT263 (Navitoclax).

Peptides of the present disclosure may, in particular, be used as a post-treatment with an additional therapeutic in subjects likely to experience metastasis or cancer recurrence from dormant cells.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. In embodiments, about means within a standard deviation using measurements generally acceptable in the art. In embodiments, about means a range extending to +/−10% of the specified value. In embodiments, about includes the specified value. Unless otherwise indicated, the word “or” in the specification and claims is considered to be the inclusive “or” rather than the exclusive or, and indicates at least one of and any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The subject invention provides a dominant negative protein comprising a leucine zipper domain having 90% or greater homology to any one of SEQ ID NOs: 7-10, 50, 52, 54, 56, 58, or 81-106.

In some embodiments, the leucine zipper domain consists essentially of any one of SEQ ID NOs: 7-10, 50, 52, 54, 56, 58, or 81-106.

In some embodiments, a cell penetrating peptide is linked directly or indirectly to the leucine zipper domain.

In some embodiments, the cell penetrating peptide is penetratin 1.

In some embodiments, the dominant negative protein consists essentially of any one of SEQ ID NOs: 51, 53, 55, 57 or 59-63.

The subject invention also provides a dominant negative protein comprising a leucine zipper domain having 90% or greater homology to any one of SEQ ID NOs: 3-6, 65, 67, 69, 71, 73, 75, or 77-80.

In some embodiments, the leucine zipper domain consists essentially of any one of SEQ ID NOs: 3-6, 65, 67, 69, 71, 73, 75, or 77-80.

In some embodiments, a cell penetrating peptide is linked directly or indirectly to the leucine zipper domain.

In some embodiments, the cell penetrating peptide is penetratin 1.

In some embodiments, the dominant negative protein consists essentially of any one of SEQ ID NOs: 66, 68, 70, 72, 74, or 76-80.

The subject invention also provides a polynucleotide comprising a sequence encoding any one of the dominant negative proteins described herein.

The subject invention also provides a composition comprising any one of the dominant negative proteins described herein and a pharmaceutically acceptable excipient.

In some embodiments, the composition further comprises a chemotherapeutic.

The subject invention also provides a method of decreasing activity or viability of a neoplastic cell, comprising contacting the neoplastic cell with any one of the dominant negative proteins described herein for a time and under conditions sufficient to cause a decrease in activity or viability of the neoplastic cell.

The subject invention also provides a method of treating cancer in a subject, comprising administering to the subject an effective amount of any one of the dominant negative proteins described herein for a time sufficient to treat a cancer in a subject.

In some embodiments, the method further comprises concurrently or during the same course of treatment administering gamma radiation or a chemotherapeutic to the subject.

In some embodiments, the chemotherapeutic comprises paclitaxel, chloroquine, doxorubicin, nab-paclitaxel, abraxane, docetaxel, 10-deacetylbaccatin III, baccatin III, paclitaxel C, 7-epipaclitaxel, daunorubicin, epirubicin, idarubicin, mitoxantrone, valrubicin, 5-Azacytidine (Azacitidine), 5-Aza-2′-deoxycytidine (Decitabine), Guadecitabine, or ABT263 (Navitoclax).

In some embodiments, the method further comprises inhibiting metastasis of the cancer, inhibiting recurrence of the cancer from dormant cancer cells, or both, in the subject.

The subject invention also provides any one of the dominant negative proteins described herein, a polynucleotide encoding any one of the dominant negative proteins described herein, or a composition comprising any one of the dominant negative proteins described herein, for use in treating cancer in a subject.

The subject invention also provides any one of the dominant negative proteins described herein, a polynucleotide encoding any one of the dominant negative proteins described herein, or a composition comprising any one of the dominant negative proteins described herein, for use in the manufacture of a medicament for treating cancer in a subject.

The subject invention also provides a non-naturally occurring composition comprising a peptide, wherein the peptide comprises a CCAAT/enhancer-binding protein delta (CEBPD) leucine zipper domain comprising one to four CEBPD repeat portions, wherein each CEBPD repeat portion has at least 70% sequence identity to any one of SEQ ID NOs: 7-10. Preferably the CEBPD repeat portion is a heptad repeat.

In some embodiments, the peptide comprises two, three, or four CEBPD repeat portions.

In some embodiments, a CEBPD repeat portion has a different amino acid sequence from another CEBPD repeat portion in the domain, preferably wherein each CEBPD repeat portion has a different amino acid sequence from any other CEBPD repeat portion in the CEBPD leucine zipper domain.

In some embodiments, a CEBPD repeat portion has the same amino acid sequence as another CEBPD repeat portion in the CEBPD leucine zipper domain.

In some embodiments, the peptide comprises more than one CEBPD repeat portion and the portions are in tandem.

In some embodiments, at least one CEBPD repeat portion has an amino acid sequence of any one of SEQ ID NOs: 7-10 with an amino acid substitution at one or two positions.

In some embodiments, at least one CEBPD repeat portion has an amino acid sequence of any one of SEQ ID NOs: 7-10 with an amino acid substitution at the first position and/or sixth position. Preferably the CEBPD repeat portion is a heptad repeat with an amino acid substitution at position g (i.e., the first position) and/or at position e (i.e., the sixth position).

In some embodiments, two separate molecules of the peptide form 1 to 6 repulsive charge interactions between their CEBPD repeat portions.

In some embodiments, a molecule of the peptide and a molecule of a protein comprising a wild-type Activating Transcription Factor 5 (ATF5) leucine zipper domain comprising SEQ ID NO: 107 form 1 to 5 attractive charge interactions between the CEBPD repeat portion of the peptide and the wild-type ATF5leucine zipper domain of the protein.

In some embodiments, a molecule of the peptide and a molecule of a protein comprising a wild-type CEBPD leucine zipper domain comprising SEQ ID NO: 108 form 1 to 5 attractive charge interactions between the CEBPD repeat portion of the peptide and the wild-type CEBPD leucine zipper domain of the protein.

In some embodiments, a molecule of the peptide and a molecule of a protein comprising a wild-type CCAAT/enhancer-binding protein beta (CEBPB) leucine zipper domain comprising SEQ ID NO: 109 form 1 to 5 attractive charge interactions between the CEBPD repeat portion of the peptide and the wild-type CEBPB leucine zipper domain of the protein.

In some embodiments, at least one CEBPD repeat portion has at least 70% sequence identity to any one of SEQ ID NOs: 7-10 and the fifth amino acid of the at least one CEBPD repeat portion is a leucine (L) residue. Preferably the CEBPD repeat portion is a heptad repeat with a leucine (L) residue at position d.

In some embodiments, at least one CEBPD repeat portion has at least 70% sequence identity to SEQ ID NO: 7, and the first amino acid is other than a lysine (K) residue.

In some embodiments, the first amino acid of the at least one CEBPD repeat portion is a negatively charged amino acid, preferably an aspartic acid (D) residue or a glutamic acid (E) residue.

In some embodiments, at least one CEBPD repeat portion has at least 70% sequence identity to SEQ ID NO: 7, and the sixth amino acid is other than a serine(S) residue.

In some embodiments, the sixth amino acid of the at least one CEBPD repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPD repeat portion has at least 70% sequence identity to SEQ ID NO: 8, and the first amino acid is other than a glutamic acid (E) residue.

In some embodiments, the first amino acid of the at least one CEBPD repeat portion is a positively charged amino acid, preferably a lysine (K) residue or an arginine (R) residue.

In some embodiments, at least one CEBPD repeat portion has at least 70% sequence identity to SEQ ID NO: 8, and the sixth amino acid is other than a histidine (H) residue.

In some embodiments, the sixth amino acid of the at least one CEBPD repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPD repeat portion has at least 70% sequence identity to SEQ ID NO: 9, and the first amino acid is other than an arginine (R) residue.

In some embodiments, wherein the first amino acid of the at least one CEBPD repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPD repeat portion has at least 70% sequence identity to SEQ ID NO: 9, and the sixth amino acid is other than a threonine (T) residue.

In some embodiments, the sixth amino acid of the at least one CEBPD repeat portion is a negatively charged amino acid, preferably an aspartic acid (D) residue or a glutamic acid (E) residue; or a positively charged amino acid, preferably a lysine (K) residue or an arginine (R) residue.

In some embodiments, the CEBPD leucine zipper domain has at least 90% sequence identity to any one of SEQ ID NOs: 7-10, 50, 52, 54, 56, 58, or 81-106.

In some embodiments, the peptide further comprises a cell-penetrating peptide portion.

In some embodiments, the cell-penetrating peptide portion is linked directly or indirectly to the CEBPD leucine zipper domain.

In some embodiments, the cell-penetrating peptide portion comprises a RL16 sequence or variant thereof, penetratin 1 sequence or variant thereof, transportan sequence or variant thereof, pIS1 sequence or variant thereof, Tat (48-60) sequence or variant thereof, pVEC sequence or variant thereof, a model amphipathic peptide (MAP) or variant thereof, or a membrane translocating sequence (MTS) or a variant thereof.

In some embodiments, the cell-penetrating peptide portion is N-terminal of the CEBPD leucine zipper domain.

In some embodiments, the peptide is a truncated dominant negative CEBPD protein.

In some embodiments, the peptide is non-naturally occurring.

In some embodiments, the CEBPD leucine zipper domain is 7-35, 7-28, 7-21, or 7-14 amino acids in length.

In some embodiments, the peptide is 7-100, 7-75, 7-50, or 7-40 amino acids in length.

In some embodiments, the peptide comprises SEQ ID NOs: 51, 53, 55, 57 or 59-63.

The subject invention also provides a non-naturally occurring composition comprising a peptide, wherein the peptide comprises a CCAAT/enhancer-binding protein beta (CEBPB) leucine zipper domain comprising one to four CEBPB repeat portions, wherein each CEBPB repeat portion has at least 70% sequence identity to any one of SEQ ID NOs: 3-6. Preferably the CEBPB repeat portion is a heptad repeat.

In some embodiments, the peptide comprises two, three, or four CEBPB repeat portions.

In some embodiments, a CEBPB repeat portion has a different amino acid sequence from another CEBPB repeat portion in the domain, preferably wherein each CEBPB repeat portion has a different amino acid sequence from any other CEBPB repeat portion in the CEBPB leucine zipper domain.

In some embodiments, a CEBPB repeat portion has the same amino acid sequence as another CEBPB repeat portion in the CEBPB leucine zipper domain.

In some embodiments, the peptide comprises more than one CEBPB repeat portion and the portions are in tandem.

In some embodiments, at least one CEBPB repeat portion has an amino acid sequence of any one of SEQ ID NOs: 3-6 with an amino acid substitution at one or two positions.

In some embodiments, at least one CEBPB repeat portion has an amino acid sequence of any one of SEQ ID NOs: 3-6 with an amino acid substitution at the first position and/or sixth position. Preferably the CEBPB repeat portion is a heptad repeat with an amino acid substitution at position g (i.e., the first position) and/or at position e (i.e., the sixth position).

In some embodiments, two separate molecules of the peptide form 1 to 6 repulsive charge interactions between their CEBPB repeat portions.

In some embodiments, a molecule of the peptide and a molecule of a protein comprising a wild-type Activating Transcription Factor 5 (ATF5) leucine zipper domain comprising SEQ ID NO: 107 form 1 to 5 attractive charge interactions between the CEBPB repeat portion of the peptide and the wild-type ATF5 leucine zipper domain of the protein.

In some embodiments, a molecule of the peptide and a molecule of a protein comprising a wild-type CEBPD leucine zipper domain comprising SEQ ID NO: 108 form 1 to 5 attractive charge interactions between the CEBPB repeat portion of the peptide and the wild-type CEBPD leucine zipper domain of the protein.

In some embodiments, a molecule of the peptide and a molecule of a protein comprising a wild-type CCAAT/enhancer-binding protein beta (CEBPB) leucine zipper domain comprising SEQ ID NO: 109 form 1 to 5 attractive charge interactions between the CEBPB repeat portion of the peptide and the wild-type CEBPB leucine zipper domain of the protein.

In some embodiments, at least one CEBPB repeat portion has at least 70% sequence identity to any one of SEQ ID NOs: 3-6 and the fifth amino acid of the at least one CEBPB repeat portion is a leucine (L) residue. Preferably the CEBPB repeat portion is a heptad repeat with a leucine (L) residue at position d.

In some embodiments, at least one CEBPB repeat portion has at least 70% sequence identity to SEQ ID NO: 3, and the first amino acid is other than a lysine (K) residue.

In some embodiments, the first amino acid of the at least one CEBPB repeat portion is a negatively charged amino acid, preferably an aspartic acid (D) residue or a glutamic acid (E) residue.

In some embodiments, at least one CEBPB repeat portion has at least 70% sequence identity to SEQ ID NO: 3, and the sixth amino acid is other than a threonine (T) residue.

In some embodiments, the sixth amino acid of the at least one CEBPB repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPB repeat portion has at least 70% sequence identity to SEQ ID NO: 4, and the first amino acid is other than a glutamic acid (E) residue.

In some embodiments, the first amino acid of the at least one CEBPB repeat portion is a positively charged amino acid, preferably a lysine (K) residue or an arginine (R) residue.

In some embodiments, at least one CEBPB repeat portion has at least 70% sequence identity to SEQ ID NO: 4, and the sixth amino acid is other than a glutamine (Q) residue.

In some embodiments, the sixth amino acid of the at least one CEBPB repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPB repeat portion has at least 70% sequence identity to SEQ ID NO: 5, and the first amino acid is other than a lysine (K) residue.

In some embodiments, the first amino acid of the at least one CEBPB repeat portion is a negatively charged amino acid, preferably an aspartic acid (D) residue or a glutamic acid (E) residue; or a positively charged amino acid, preferably a lysine (K) residue or an arginine (R) residue.

In some embodiments, at least one CEBPB repeat portion has at least 70% sequence identity to SEQ ID NO: 5, and the sixth amino acid is other than a serine(S) residue.

In some embodiments, the sixth amino acid of the at least one CEBPB repeat portion is a negatively charged amino acid, preferably an aspartic acid (D) residue or a glutamic acid (E) residue; or a positively charged amino acid, preferably a lysine (K) residue or an arginine (R) residue.

In some embodiments, the CEBPB leucine zipper domain has at least 90% sequence identity to any one of SEQ ID NOs: 3-6, 65, 67, 69, 71, 73, 75, 77-80, or 114.

In some embodiments, the peptide further comprises a cell-penetrating peptide portion.

In some embodiments, the cell-penetrating peptide portion is linked directly or indirectly to the CEBPB leucine zipper domain.

In some embodiments, the cell-penetrating peptide portion comprises a RL16 sequence or variant thereof, penetratin 1 sequence or variant thereof, transportan sequence or variant thereof, pIS1 sequence or variant thereof, Tat (48-60) sequence or variant thereof, pVEC sequence or variant thereof, a model amphipathic peptide (MAP) or variant thereof, or a membrane translocating sequence (MTS) or a variant thereof.

In some embodiments, the cell-penetrating peptide portion is N-terminal of the CEBPB leucine zipper domain.

In some embodiments, the peptide is a truncated dominant negative CEBPB protein.

In some embodiments, the peptide is non-naturally occurring.

In some embodiments, the CEBPB leucine zipper domain is 7-35, 7-28, 7-21, or 7-14 amino acids in length.

In some embodiments, the peptide is 7-100, 7-75, 7-50, or 7-40 amino acids in length.

In some embodiments, the peptide comprises SEQ ID NOs: 66, 68, 70, 72, 74, 76-80, or 114.

In some embodiments, the compositions further comprise a pharmaceutically acceptable excipient.

In some embodiments, the compositions further comprise a chemotherapeutic agent.

In some embodiments, the chemotherapeutic comprises paclitaxel, chloroquine, doxorubicin, nab-paclitaxel, abraxane, docetaxel, 10-deacetylbaccatin III, baccatin III, paclitaxel C, 7-epipaclitaxel, daunorubicin, epirubicin, idarubicin, mitoxantrone, valrubicin, 5-Azacytidine (Azacitidine), 5-Aza-2′-deoxycytidine (Decitabine), Guadecitabine, or ABT263 (Navitoclax).

The subject invention also provides a method of decreasing activity or viability of a neoplastic cell, comprising delivering the any one of the compositions described herein to the neoplastic cell.

In some embodiments, the neoplastic cell is a cancer cell, preferably a cancer cell, preferably breast cancer cell, colon cancer cell, prostate cancer cell, bladder cancer cell, soft-tissue sarcoma cell, an advanced lung cancer cell, lung cancer cell, non-small cell lung cancer cell, small cell lung cancer cell, mesothelioma cell, esophageal cancer cell, liver cancer cell, renal cell cancer cell, melanoma cell, skin cancer cell, basal cell skin cancer cell, squamous cell skin cancer cell, or a squamous cell carcinoma cell.

The subject invention also provides a method of treating cancer in a subject, comprising administering to the subject an effective amount of any one of the compositions described herein.

In some embodiments, the method further comprises inhibiting metastasis of the cancer, inhibiting recurrence of the cancer from dormant cancer cells, or both, in the subject.

In some embodiments, the cancer is a breast cancer, estrogen receptor positive (ER+) breast cancer, triple-negative breast cancer (TNBC), colon cancer, prostate cancer, bladder cancer, soft-tissue sarcoma, an advanced lung cancer, lung cancer, non-small cell lung cancer, small cell lung cancer, mesothelioma, esophageal cancer, liver cancer, renal cell cancer, melanoma, skin cancer, basal cell skin cancer, squamous cell skin cancer, squamous cell carcinoma of the head and neck, or leukemia.

The subject invention also provides a polynucleotide molecule encoding any one of the peptides described herein.

The subject invention also provides any one of the compositions described herein or any one of the polynucleotides described herein for use in treating cancer in a subject.

The subject invention also provides any one of the compositions described herein or any one of the polynucleotides described herein for use in the manufacture of a medicament for treating cancer in a subject.

For the foregoing embodiments, each embodiment disclosed herein is contemplated as being applicable to each of the other disclosed embodiment.

As used herein, all headings are simply for organization and are not intended to limit the disclosure in any manner. The content of any individual section may be equally applicable to all sections. All combinations of the various elements disclosed herein are within the scope of the invention.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only. Specifically, the DN proteins, methods, compositions, and polynucleotides herein disclosed are further illustrated in the following examples, which are provided by way of illustration and are not intended to be limiting.

### EXAMPLES

### Example 1—Exemplary Truncated DN Constructs for Interference with One or More Functions of CEBPB, and/or CEBPD

This Example describes exemplary amino acid sequences of truncated DN constructs for interference with one or more functions of CEBPB and/or CEBPD, such as signaling including one or more of these proteins, and for treatment of cancers.

The amino acid sequences shown in Table 3 are examples of DN constructs including amino acid sequence variants of DN CEBPD protein configured to interfere with one or more functions of CEBPB or CEBPD, among other proteins described herein, such as signaling including one or more of these proteins, and for treatment of cancers.

TABLE 3
Amino acid sequences of DN CEBPD constructs

| Construct | Amino acid sequence |
|---|---|
| Dpep | RQIKIWFQNRRMKWKKLVELSAENEKLHQRVEQLTRDLA |
| | GLRQFFK (SEQ ID NO: 48) |
| D-1234 | KLVELSAENEKLHQRVEQLTRDLAGLRQ |
| | (SEQ ID NO: 49) |
| D-123 | KLVELSAENEKLHQRVEQLTR (SEQ ID NO: 50) |
| CP-D-123 | RQIKIWFQNRRMKWKKLVELSAENEKLHQRVEQLTR |
| | (SEQ ID NO: 51) |
| D-234 | ENEKLHQRVEQLTRDLAGLRQ (SEQ ID NO: 52) |
| CP-D-234 | RQIKIWFQNRRMKWKKENEKLHQRVEQLTRDLAGLRQ |
| | (SEQ ID NO: 53) |
| D-12 | KLVELSAENEKLHQ (SEQ ID NO: 54) |
| CP-D-12 | RQIKIWFQNRRMKWKKLVELSAENEKLHQ |
| | (SEQ ID NO: 55) |
| D-23 | ENEKLHQRVEQLTR (SEQ ID NO: 56) |
| CP-D-23 | RQIKIWFQNRRMKWKKENEKLHQRVEQLTR |
| | (SEQ ID NO: 57) |
| D-34 | RVEQLTRDLAGLRQ (SEQ ID NO: 58) |
| CP-D-34 | RQIKIWFQNRRMKWKKRVEQLTRDLAGLRQ |
| | (SEQ ID NO: 59) |
| D-1 | KLVELSA (SEQ ID NO: 7) |
| CP-D-1 | RQIKIWFQNRRMKWKKLVELSA (SEQ ID NO: 60) |
| D-2 | ENEKLHQ (SEQ ID NO: 8) |
| CD-D-2 | RQIKIWFQNRRMKWKKENEKLHQ (SEQ ID NO: 61) |
| D-3 | RVEQLTR (SEQ ID NO: 9) |
| CP-D-3 | RQIKIWFQNRRMKWKKRVEQLTR (SEQ ID NO: 62) |
| D-4 | DLAGLRQ (SEQ ID NO: 10) |
| CP-D-4 | RQIKIWFQNRRMKWKKDLAGLRQ (SEQ ID NO: 63) |

SEQ ID NO: 48 is an exemplary sequence of a construct referred to as Dpep wherein a penetratin sequence is followed by the CEBPD protein leucine zipper domain sequence, italicized and bold.

SEQ ID NO: 49 is an exemplary sequence of a construct referred to as D-1234 wherein the leucine zipper domain of Dpep has the c-terminal three amino acids removed. The leucine zipper domain includes four “heptad” repeats.

SEQ ID NO: 50 is an exemplary sequence of a construct referred to as D-123 having the first, second and third heptad repeats of D-1234. SEQ ID NO: 51 is an exemplary sequence of a construct referred to as CP-D-123, wherein D-123, italicized and bold, is preceded by a penetratin sequence.

SEQ ID NO: 52 is an exemplary sequence of a construct referred to as D-234 having the second, third, and fourth heptad repeats of D-1234. SEQ ID NO: 53 is an exemplary sequence of a construct referred to as CP-D-234, wherein D-234, italicized and bold, is preceded by a penetratin sequence.

SEQ ID NO: 54 is an exemplary sequence of a construct referred to as D-12 having the first and second heptad repeats of D-1234. SEQ ID NO: 55 is an exemplary sequence of a construct referred to as CP-D-12, wherein D-12, italicized and bold, is preceded by a penetratin sequence.

SEQ ID NO: 56 is an exemplary sequence of a construct referred to as D-23 having the second and third heptad repeats of D-1234. SEQ ID NO: 57 is an exemplary sequence of a construct referred to as CP-D-23, wherein D-23, italicized and bold, is preceded by a penetratin sequence.

SEQ ID NO: 58 is an exemplary sequence of a construct referred to as D-34 having the third and fourth heptad repeats of the D-1234. SEQ ID NO: 59 is an exemplary sequence of a construct referred to as CP-D-34, wherein D-34, italicized and bold, is preceded by a penetratin sequence.

SEQ ID NO: 7 is an exemplary sequence of a construct referred to as D-1 having the first heptad repeat of D-1234. SEQ ID NO: 60 is an exemplary sequence of a construct referred to as CP-D-1, wherein D-1, italicized and bold, is preceded by a penetratin sequence.

SEQ ID NO: 8 is an exemplary sequence of a construct referred to as D-2 having the second heptad repeat of D-1234. SEQ ID NO: 61 is an exemplary sequence of a construct referred to as CP-D-2, wherein D-2, italicized and bold, is preceded by a penetratin sequence.

SEQ ID NO: 9 is an exemplary sequence of a construct referred to as D-3 having the third heptad repeat of D-1234. SEQ ID NO: 62 is an exemplary sequence of a construct referred to as CP-D-3, wherein D-3, italicized and bold, is preceded by a penetratin sequence.

SEQ ID NO: 10 is an exemplary sequence of a construct referred to as D-4 having the fourth heptad repeat of D-1234. SEQ ID NO: 63 is an exemplary sequence of a construct referred to as CP-D-4, wherein D-4, italicized and bold, is preceded by a penetratin sequence.

As D-1234 includes four heptad repeats, it may be referred to herein as a “4-box peptide.” Similarly, D-123 and D-234 may be referred to herein as “3-box peptides;” D12, D-23, D-34 as “2-box peptides;” and D-1, D-2, D-3, and D-4 as “1-box peptides.”

The amino acid sequences shown in Table 4 are examples of DN constructs including amino acid sequence variants of DN CEBPB protein configured to interfere with one or more functions of CEBPB or CEBPD, among other proteins described herein, such as signaling including one or more of these proteins, and for treatment of cancers.

TABLE 4
Amino acid sequences of DN CEBPB constructs

| Construct | Amino acid sequence |
|---|---|
| Bpep | RQIKIWFQNRRMKWKKLETQHKVLELTAENERLQKKVEQL |
| | SRELSTLRNLFKQL (SEQ ID NO: 64) |
| B-1234 | KVLELTAENERLQKKVEQLSRELSTLRN |
| | (SEQ ID NO: 65) |
| CP-B-1234 | RQIKIWFQNRRMKWKKVLELTAENERLQKKVEQLSRELST |
| | LRN (SEQID NO: 66) |
| B-123 | KVLELTAENERLQKKVEQLSR (SEQ ID NO: 67) |
| CP-B-123 | RQIKIWFQNRRMKWKKVLELTAENERLQKKVEQLSR |
| | (SEQ ID NO: 68) |
| B-234 | ENERLQKKVEQLSRELSTLRN (SEQ ID NO: 69) |
| CP-B-234 | RQIKIWFQNRRMKWKKENERLQKKVEQLSRELSTLRN |
| | (SEQ ID NO: 70) |
| B-12 | KVLELTAENERLQK (SEQ ID NO: 71) |
| CP-B-12 | RQIKIWFQNRRMKWKKVLELTAENERLQK |
| | (SEQ ID NO: 72) |
| B-23 | ENERLQKKVEQLSR (SEQ ID NO: 73) |
| CP-B-23 | RQIKIWFQNRRMKWKKENERLQKKVEQLSR |
| | (SEQ ID NO: 74) |
| B-34 | KVEQLSRELSTLRN (SEQ ID NO: 75) |
| CP-B-34 | RQIKIWFQNRRMKWKKVEQLSRELSTLRN |
| | (SEQ ID NO: 76) |
| B-1 | KVLELTA (SEQ ID NO: 3) |
| CP-B-1 | RQIKIWFQNRRMKWKKVLELTA (SEQ ID NO: 77) |
| B-2 | ENERLQK (SEQ ID NO: 4) |
| CP-B-2 | RQIKIWFQNRRMKWKKENERLQK (SEQ ID NO: 78) |
| B-3 | KVEQLSR (SEQ ID NO: 5) |
| CP-B-3 | RQIKIWFQNRRMKWKKVEQLSR (SEQ ID NO: 79) |
| B-4 | ELSTLRN (SEQ ID NO: 6) |
| CP-B-4 | RQIKIWFQNRRMKWKKELSTLRN (SEQ ID NO: 80) |

SEQ ID NO: 64 is an exemplary sequence of a construct referred to as Bpep wherein a penetratin sequence is followed by the CEBPB protein leucine zipper domain sequence, italicized and bold.

SEQ ID NO: 65 is an exemplary sequence of a construct referred to as B-1234, which is a segment of the leucine zipper domain of Bpep. The leucine zipper domain of B-1234 includes four “heptad” repeats. SEQ ID NO: 66 is an exemplary sequence of a construct referred to as CP-B-1234, wherein B-1234, italicized and bold, is preceded by a penetratin sequence.

SEQ ID NO: 67 is an exemplary sequence of a construct referred to as B-123 having the first, second and third heptad repeats of B-1234. SEQ ID NO: 68 is an exemplary sequence of a construct referred to as CP-B-123, wherein B-123, italicized and bold, is preceded by a penetratin sequence.

SEQ ID NO: 69 is an exemplary sequence of a construct referred to as B-234 having the second, third, and fourth heptad repeats of B-1234. SEQ ID NO: 70 is an exemplary sequence of a construct referred to as CP-B-234, wherein B-234, italicized and bold, is preceded by a penetratin sequence.

SEQ ID NO: 71 is an exemplary sequence of a construct referred to as B-12 having the first and second heptad repeats of B-1234. SEQ ID NO: 72 is an exemplary sequence of a construct referred to as CP-B-12, wherein B-12, italicized and bold, is preceded by a penetratin sequence.

SEQ ID NO: 73 is an exemplary sequence of a construct referred to as B-23 having the second and third heptad repeats of B-1234. SEQ ID NO: 74 is an exemplary sequence of a construct referred to as CP-B-23, wherein B-23, italicized and bold, is preceded by a penetratin sequence.

SEQ ID NO: 75 is an exemplary sequence of a construct referred to as B-34 having the third and fourth heptad repeats of the B-1234. SEQ ID NO: 76 is an exemplary sequence of a construct referred to as CP-B-34, wherein B-34, italicized and bold, is preceded by a penetratin sequence.

SEQ ID NO: 3 is an exemplary sequence of a construct referred to as B-1 having the first heptad repeat of B-1234. SEQ ID NO: 77 is an exemplary sequence of a construct referred to as CP-B-1, wherein B-1, italicized and bold, is preceded by a penetratin sequence.

SEQ ID NO: 4 is an exemplary sequence of a construct referred to as B-2 having the second heptad repeat of B-1234. SEQ ID NO: 78 is an exemplary sequence of a construct referred to as CP-B-2, wherein B-2, italicized and bold, is preceded by a penetratin sequence.

SEQ ID NO: 5 is an exemplary sequence of a construct referred to as B-3 having the third heptad repeat of B-1234. SEQ ID NO: 79 is an exemplary sequence of a construct referred to as CP-B-3, wherein B-3, italicized and bold, is preceded by a penetratin sequence.

SEQ ID NO: 6 is an exemplary sequence of a construct referred to as B-4 having the fourth heptad repeat of B-1234. SEQ ID NO: 80 is an exemplary sequence of a construct referred to as CP-B-4, wherein B-4, italicized and bold, is preceded by a penetratin sequence.

### Example 2—Truncated DN-CEPBD Peptides Promote Apoptotic Death of HCT116 Cultures

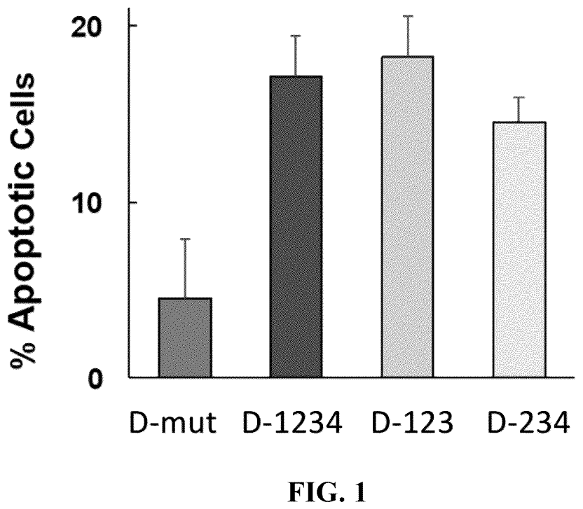

FIG. 1 is a graph reporting exemplary data showing 3-box dominant negative forms of CEBPD promote apoptotic death of cancer cells. Replicate cultures of HCT116 colon cancer cells were transfected with DNA vectors expressing D-mut (a control peptide containing only the leucine zipper domain of CEBPD with all leucines in position 5 (also referred to as position “d”) of the heptad repeats mutated to glycine to suppress leucine zipper binding activity), D-1234, D-123, and D-234. Three days later, transfected cells were scored for proportion with apoptotic nuclei. Data represent means±SEM (n=4). P values vs D-mut (T-test): D-1234, p=0.0005; D-123, p=0.0014; D-234, p=0.0003. A second independent experiment produced comparable results.

Transfection rates for each construct were assessed and are presented in Table 5:

TABLE 5
Transfection rates

| Construct | Transfection rate |
|---|---|
| D-mut | 36% |
| D1,2,3,4 | 40% |
| D1,2,3 | 44% |
| D2,3,4 | 42% |

The results show that the 3-box peptides D-123 and D-234 can cause apoptosis in HCT116 cell cultures at comparable rates to the full length D-1234 construct and have comparable rates of transfection.

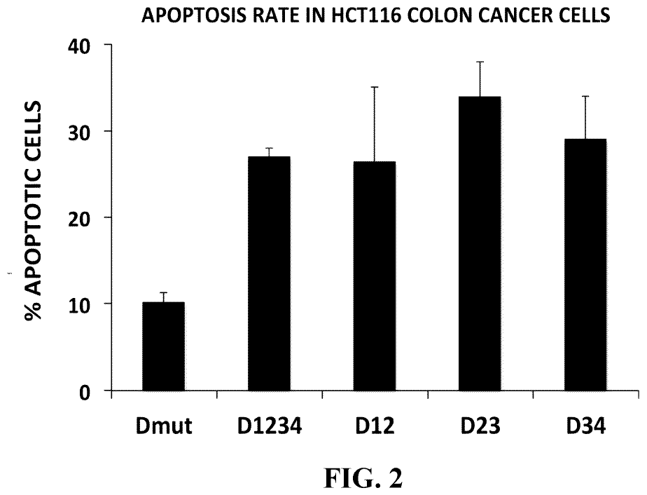

FIG. 2 is a graph reporting exemplary data showing 2-box dominant negative forms of CEBPD promote apoptotic death of cancer cells. Replicate cultures of HCT116 cells were transfected with DNA vectors expressing D-mut, D-1234, D-12, D-23, or D-34. Three days later, transfected cells were scored for proportion with apoptotic nuclei. Data represent means±SEM (n=12 from 3 independent experiments). P values vs D-mut: D-1234, p<0.0001; D-12, p=0.056; D-23, p<0.001; D-34, p=0.0008.

Transfection rates for each construct were assessed and are presented in Table 6:

TABLE 6
Mean transfection rates

| Construct | Transfection rate |
|---|---|
| D-mut | 28% |
| D-1234 | 22% |
| D-12 | 5% |
| D-23 | 9% |
| D-34 | 7% |

The results show that the 2-box peptides D-12, D-23, and D-34 are active in causing apoptosis in HCT116 cell cultures and have a lower transfection rate than D-1234.

These results support the development of truncated dominant negative forms of CEBPB and CEBPD proteins as potential cancer therapeutics.

### Example 3—CP-D-123 and CP-D-234 Reduce Cell Numbers in Cancer Cell Lines

Peptides CP-D-123 (SEQ ID NO: 51) and CP-D-234 (SEQ ID NO: 53) were commercially synthesized.

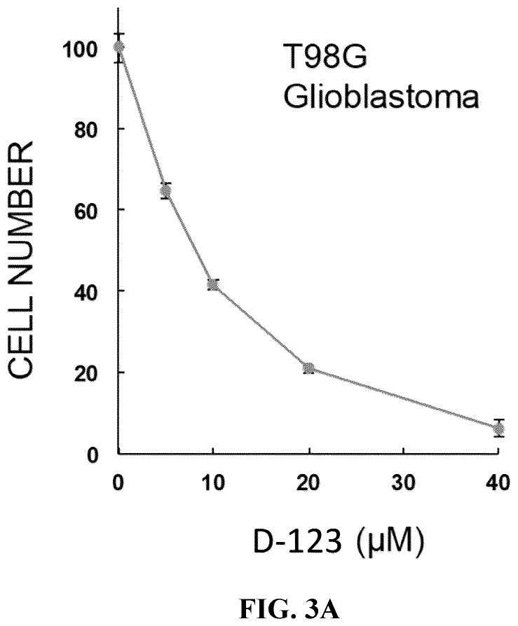

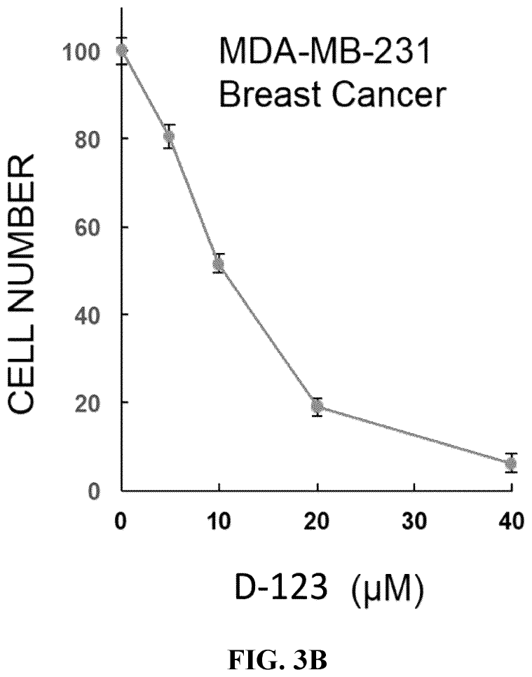

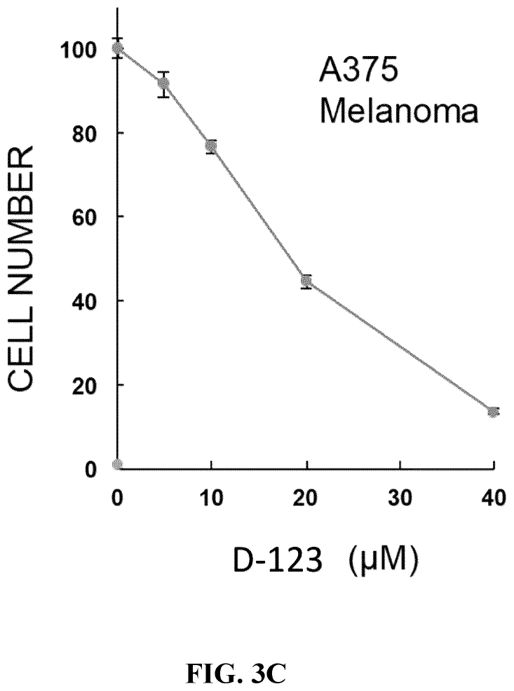

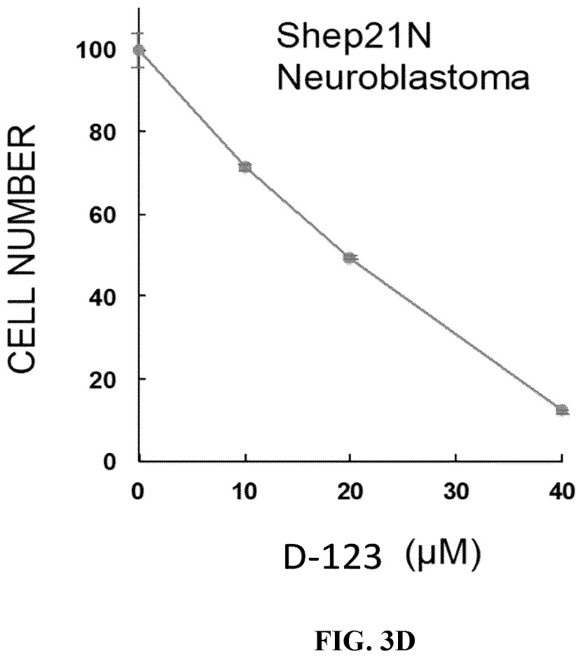

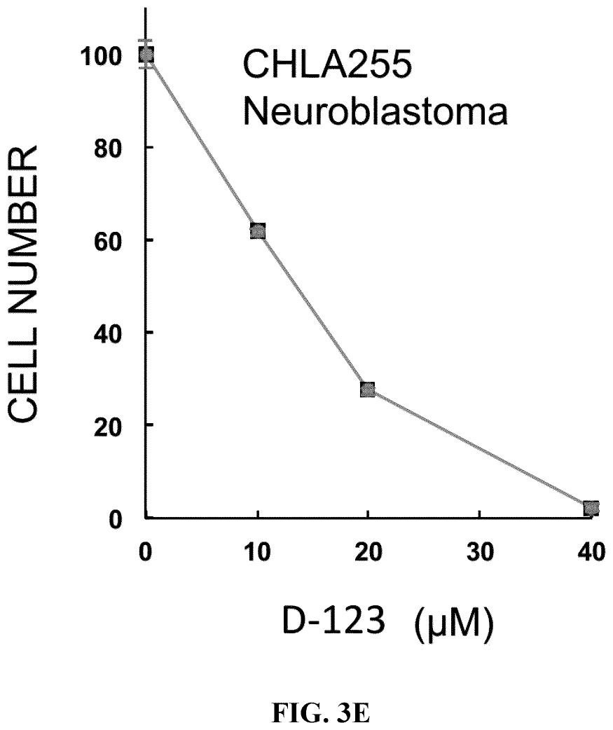

FIGS. 3A-3E are graphs reporting exemplary data showing truncated dominant negative forms of CEBPD reduce cancer cell numbers. Replicate cultures of T98G (FIG. 3A), MDA-MB-231 (FIG. 3B), A375 (FIG. 3C), Shep21N (FIG. 3D), and CHLA255 (FIG. 3E) cell lines were treated with CP-D-123 at the indicated concentrations for 6 days in culture medium containing 2% fetal bovine serum and then assessed for cell numbers. Values are given as means±SEM (N=3 replicate cultures).

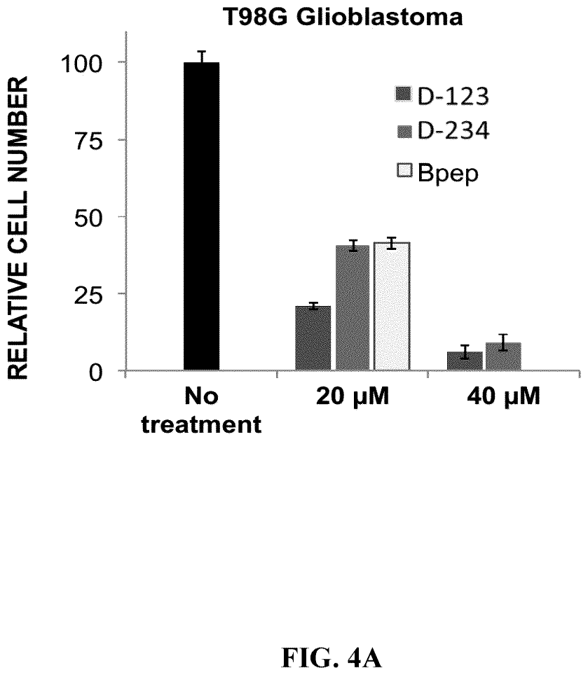

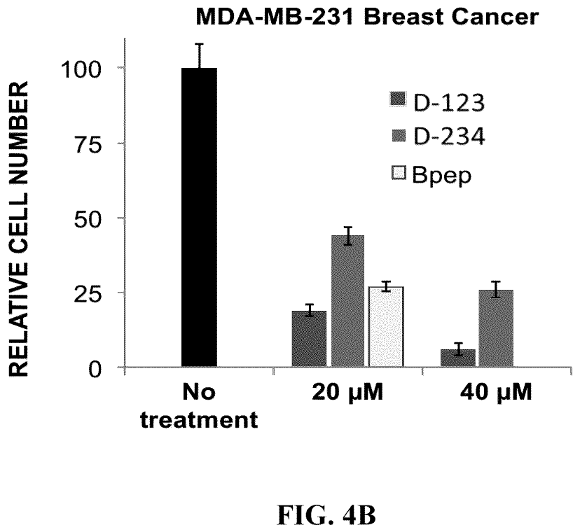

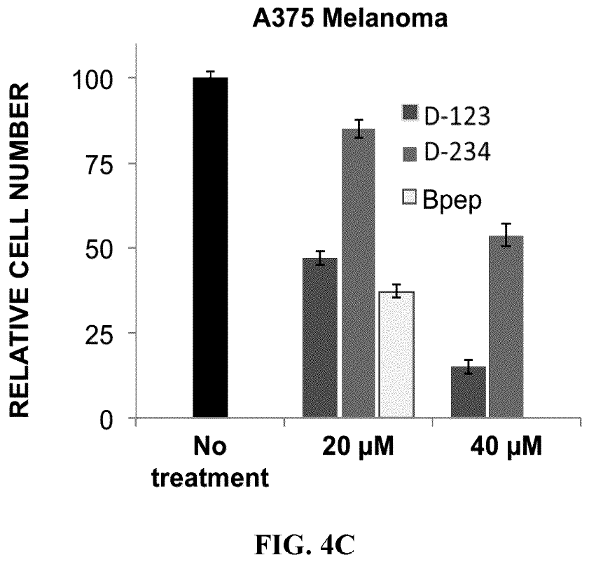

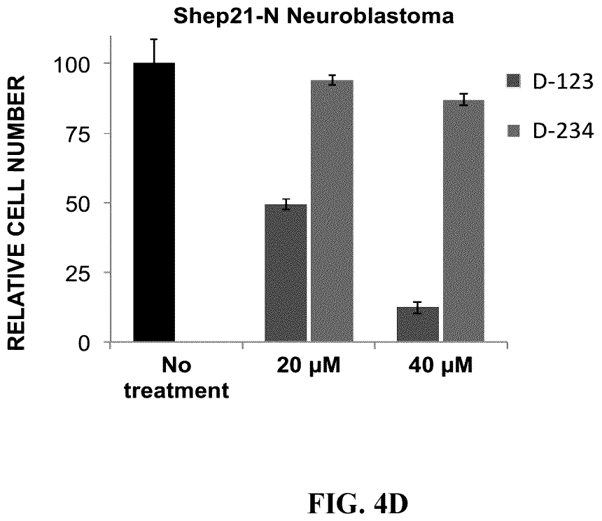

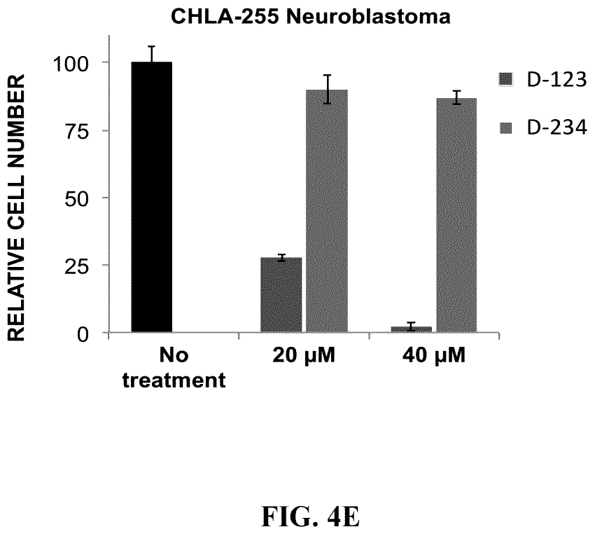

FIGS. 4A-4E are graphs reporting exemplary data showing truncated dominant negative forms of CEBPD reduce cancer cell numbers. Replicate cultures of T98G (FIG. 4A), MDA-MB-231 (FIG. 4B), A375 (FIG. 4C), Shep21N (FIG. 4D), and CHLA255 (FIG. 4E) cell lines were treated with CP-D-123, CP-D-234, and Bpep (at 20 μM in T98G, MDA-MB-231, A375 only) at the indicated concentrations for 6 days in culture medium containing 2% fetal bovine serum and then assessed for cell numbers. Values are given as means±SEM (N=3 replicate cultures).

Reduction of cell numbers with CP-D-123 is comparable to or higher than that previously published for Dpep (Zhou, et al., 2021, Cancers, 13:2504). Reduction of cell numbers with CP-D-234 is lower and less consistent in activity than CP-D-123.

### Example 4—CP-D-123 (10 mg/kg) Significantly Reduces Growth of A375 Melanoma Subcutaneous Xenografts in Nude Mice

A375 human melanoma cells were injected under the skin of the flanks of female nude mice. When tumors were detected the mice were treated intraperitoneally three times per week with either vehicle (PBS) or CP-D-123 (10 mg/kg) on the indicated days after initial cell inoculation. Three mice were treated in each treatment group. Tumor volumes were calculated with caliper measurements. Nine tumors were assessed for vehicle-treated mice and seven tumors were assessed for CP-D-123-treated mice. Tumor volumes are reported up to the time that the first animal reached the pre-determined endpoint (at least 1 tumor>1000 mm³ in volume) for euthanization.

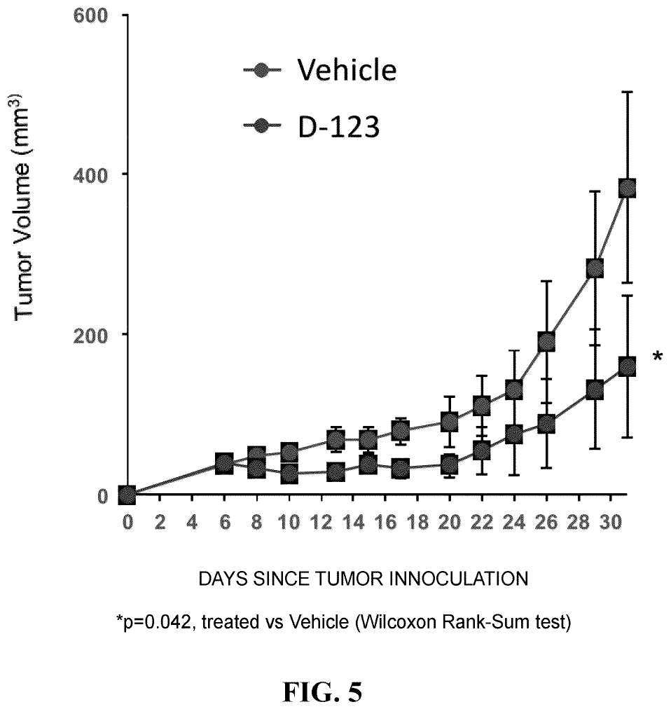

FIG. 5 is a graph reporting exemplary data showing mean tumor volumes (mm³) per treatment group (±SEM) over time (days). Tumor volume differences between treatment and vehicle groups were assessed with the Wilcoxon Rank-Sum test and found to be significant (p=0.042).

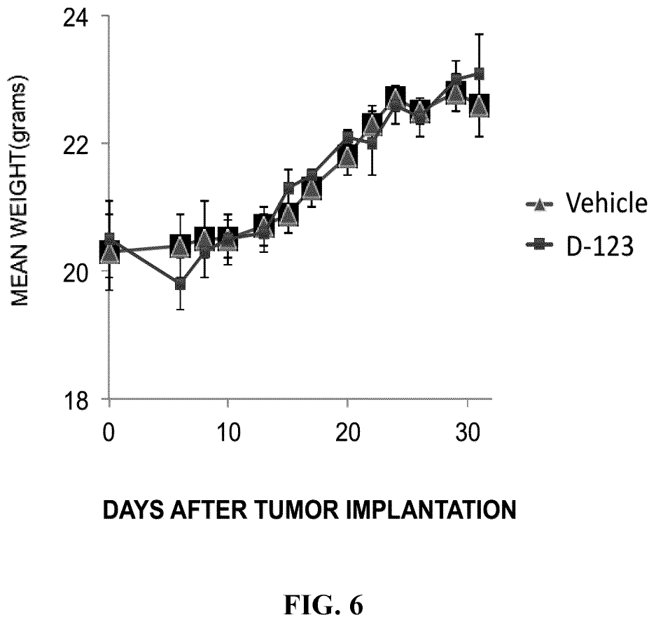

FIG. 6 is a graph reporting exemplary data showing mean animal weight (grams) per treatment group (±SEM) over time (days).

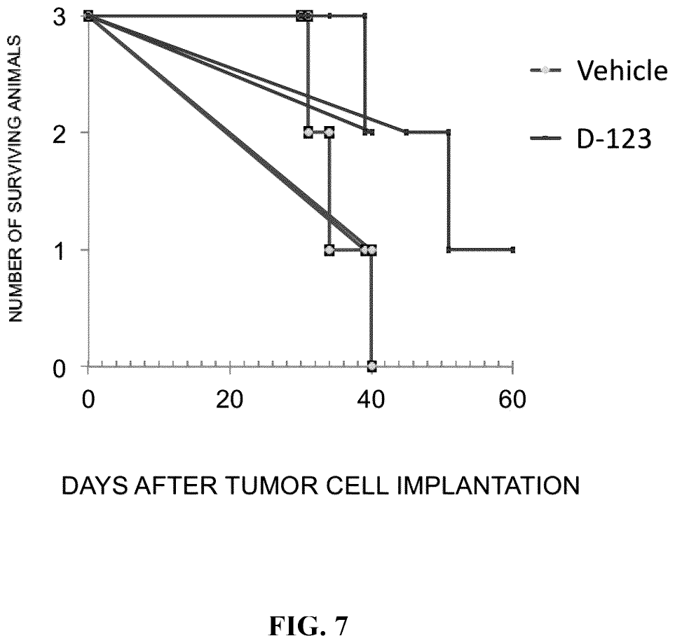

FIG. 7 is a graph reporting exemplary data showing the number of surviving animals (animals which have not reached the predetermined endpoint of the study) per treatment group over time (days) The survival study was ended at 60 days after tumor inoculation.

The data indicate administration of CP-D-123 resulted in a significant reduction in tumor growth and prolonged animal survival with no effect on animal weight. No evident side effects were noted over at least 6 weeks of treatment 3 days per week.

### Example 5—Exemplary Optimized Truncated DN Constructs

The CEBPD peptide forms homodimers as well as heterodimers with other peptides containing appropriate leucine zipper domains. Dpep, Bpep, and their truncated variants may also form homodimers, which could diminish the extent to which they interact with ATF5, CEBPD, and CEBPB and reduce their efficacy as dominant negative constructs.

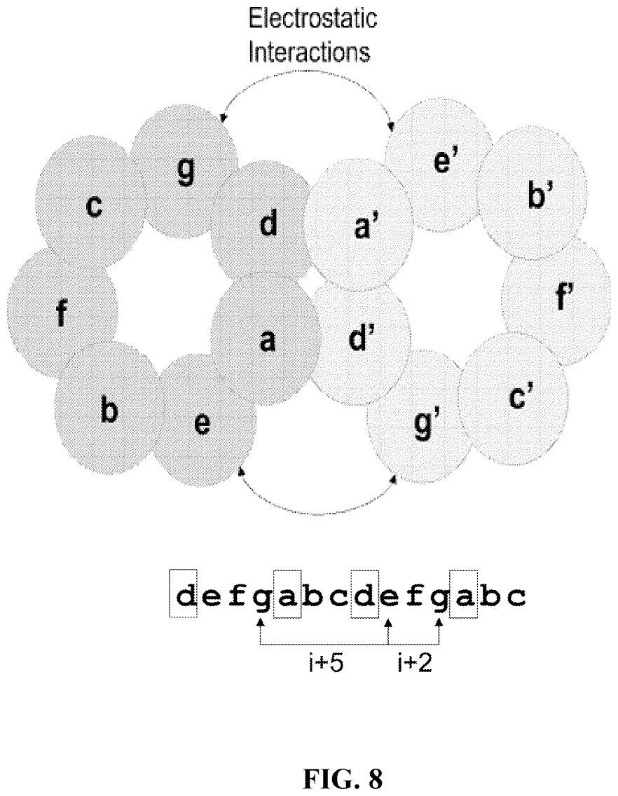

FIG. 8 shows an exemplary illustration of an end view of two interacting leucine helices in a coiled-coil structure. One heptad repeat is shown for each helix with the residues in one helix being labeled a-g and the residues in the other labeled a′-g′. Residues in the a and d positions of each coil interact to form a hydrophobic core. Residues in the e and g positions are near enough to form electrostatic interactions. Specifically, residues in the e position of one helix will interact with two residues in the corresponding g positions of the other helix, one located 5 positions toward the N terminus of the peptide (referred to as an i+5 interaction) and the other located 2 positions toward the C terminus of the peptide (referred to as an i+2 interaction). Similarly, residues in the g position of one helix will interact with two residues in the corresponding e position of the other helix, one located 2 positions toward the N terminus of the peptide (an i+2 interaction) and the other located 5 positions toward the C terminus of the peptide (an i+5 interaction). When two helices coil around one another, the i+5 interactions are closer than and predominate the i+2 interactions. Dimer formation will tend to be favored when residues of an i+5 interaction have opposite charges and disfavored when they have the same charge.

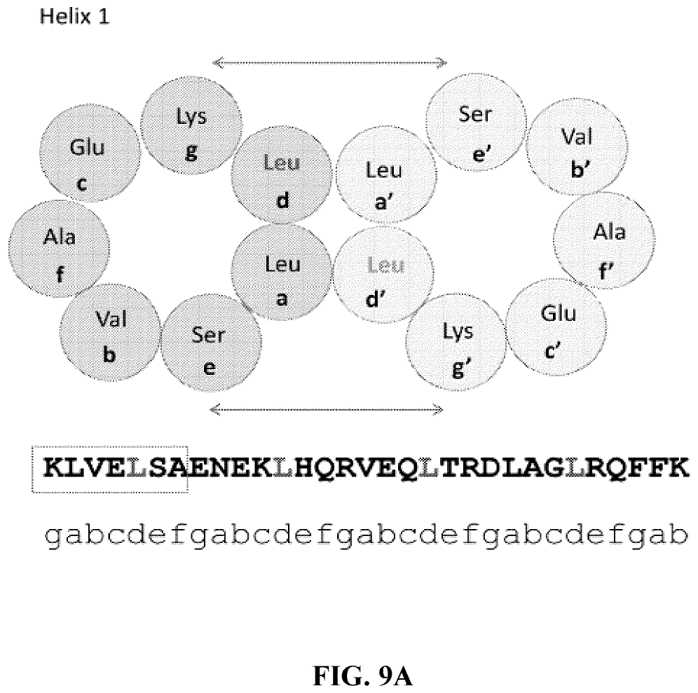

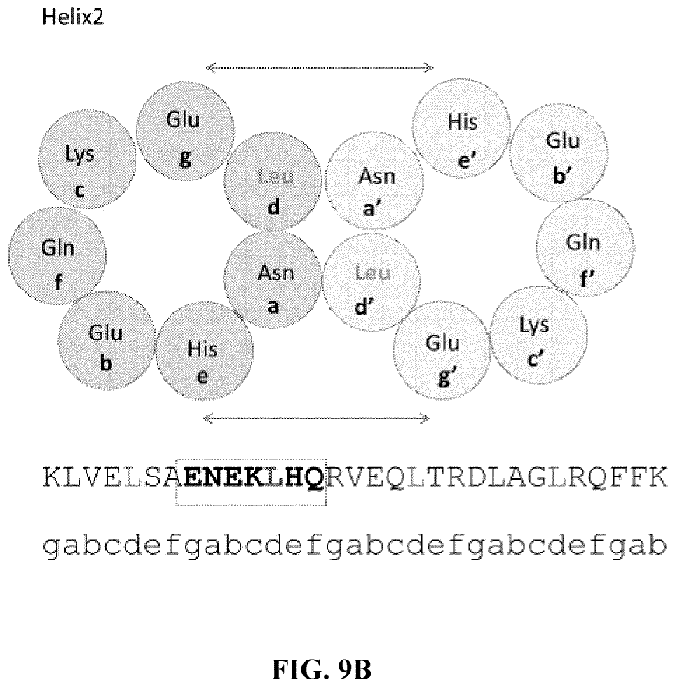

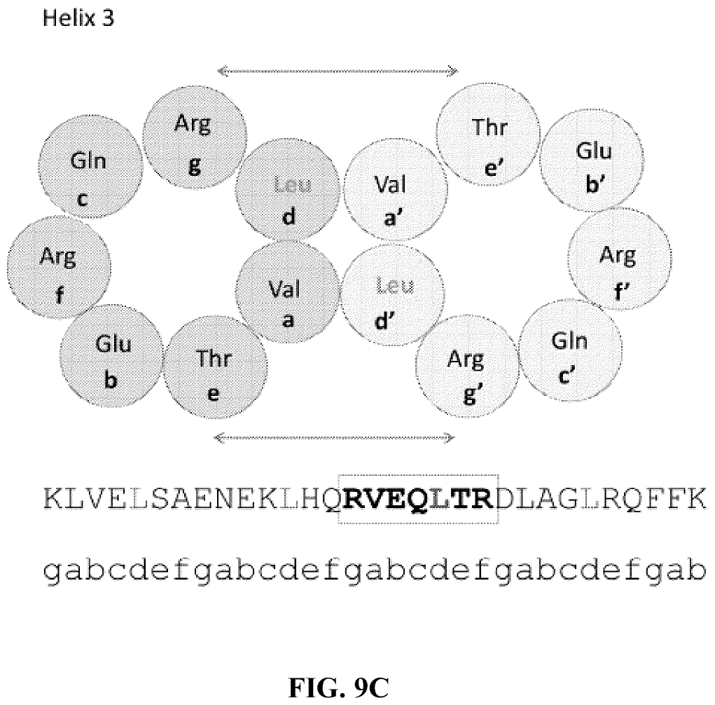

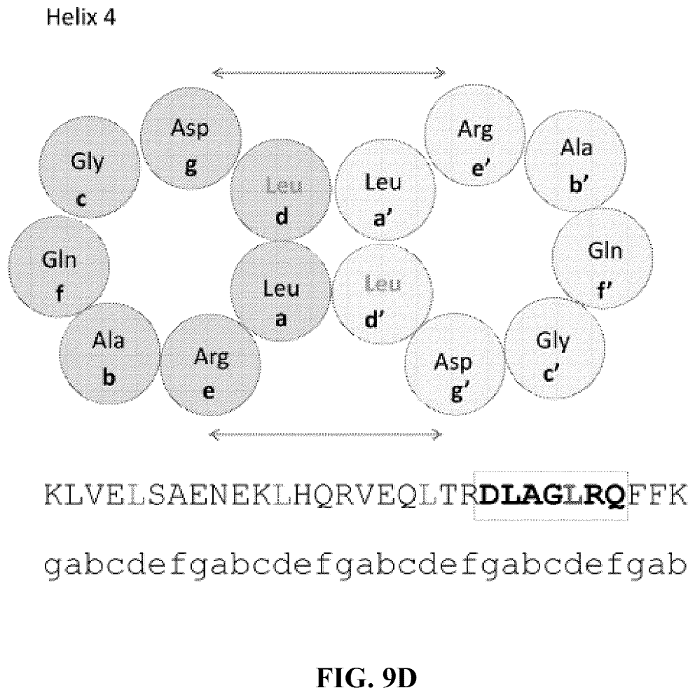

FIGS. 9A-9D show exemplary illustrations of each of the four heptad repeats of a D-1234 homodimer. The respective i+5 interactions for each repeat are indicated with arrows.

The amino acid sequences shown in Table 7 are examples of amino acid sequence variants of D-123 (SEQ ID NO: 50). In each variant, the 1^{st} and 6^{th} residue of D-123 are either both left as wild-type (“W”), both mutated to glutamic acid (“E”), or both mutated to lysine (“K”); the 8^{th} and 13^{th} residue are either both left as wild-type (“W”), both mutated to glutamic acid (“E”), or both mutated to lysine (“K”); and the 15^{th} and 20^{th} residue are either both left as wild-type (“W”), both mutated to glutamic acid (“E”), or both mutated to lysine (“K”). The 1^{st}, 6^{th}, 8^{th}, 13^{th}, 15^{th}, and 20^{th} residues are underlined.

TABLE 7
Amino acid sequences of D-123 variants

| | | SEQ ID |
|---|---|---|
| Construct | Amino acid sequence | NO: |
| D-123 | KLVELSA ENEKLHQ RVEQLTR (SEQ ID NO: 50) | 50 |
| D-123WWK | KLVELSA ENEKLHQ KVEQLKR (SEQ ID NO: 81) | 81 |
| D-123WWE | KLVELSA ENEKLHQ EVEQLER (SEQ ID NO: 82) | 82 |
| D-123WKW | KLVELSA KNEKLKQ RVEQLTR (SEQ ID NO: 83) | 83 |
| D-123WEW | KLVELSA ENEKLEQ RVEQLTR (SEQ ID NO: 84) | 84 |
| D-123KWW | KLVELKA ENEKLHQ RVEQLTR (SEQ ID NO: 85) | 85 |
| D-123EWW | ELVELEA ENEKLHQ RVEQLTR (SEQ ID NO: 86) | 86 |
| D-123WKK | KLVELSA KNEKLKQ KVEQLKR (SEQ ID NO: 87) | 87 |
| D-123WEE | KLVELSA ENEKLEQ EVEQLER (SEQ ID NO: 88) | 88 |
| D-123KWK | KLVELKA ENEKLHQ KVEQLKR (SEQ ID NO: 89) | 89 |
| D-123EWE | ELVELEA ENEKLHQ EVEQLER (SEQ ID NO: 90) | 90 |
| D-123KKW | KLVELKA KNEKLKQ RVEQLTR (SEQ ID NO: 91) | 91 |
| D-123EEW | ELVELEA ENEKLEQ RVEQLTR (SEQ ID NO: 92) | 92 |
| D-123EKW | ELVELEA KNEKLKQ RVEQLTR (SEQ ID NO: 93) | 93 |
| D-123EWK | ELVELEA ENEKLHQ KVEQLKR (SEQ ID NO: 94) | 94 |
| D-123WEK | KLVELSA ENEKLEQ KVEQLKR (SEQ ID NO: 95) | 95 |
| D-123WKE | KLVELSA KNEKLKQ EVEQLER (SEQ ID NO: 96) | 96 |
| D-123KWE | KLVELKA ENEKLHQ EVEQLER (SEQ ID NO: 97) | 97 |
| D-123KEW | KLVELKA ENEKLEQ RVEQLTR (SEQ ID NO: 98) | 98 |
| D-123KEE | KLVELKA ENEKLEQ EVEQLER (SEQ ID NO: 99) | 99 |
| D-123EKE | ELVELEA KNEKLKQ EVEQLER (SEQ ID NO: 100) | 100 |
| D-123EEK | ELVELEA ENEKLEQ KVEQLKR (SEQ ID NO: 101) | 101 |
| D-123EEE | ELVELEA ENEKLEQ EVEQLER (SEQ ID NO: 102) | 102 |
| D-123KKK | KLVELKA KNEKLKQ KVEQLKR (SEQ ID NO: 103) | 103 |
| D-123KKE | KLVELKA KNEKLKQ EVEQLER (SEQ ID NO: 104) | 104 |
| D-123KEK | KLVELKA ENEKLEQ KVEQLKR (SEQ ID NO: 105) | 105 |
| D-123EKK | ELVELEA KNEKLKQ KVEQLKR (SEQ ID NO: 106) | 106 |

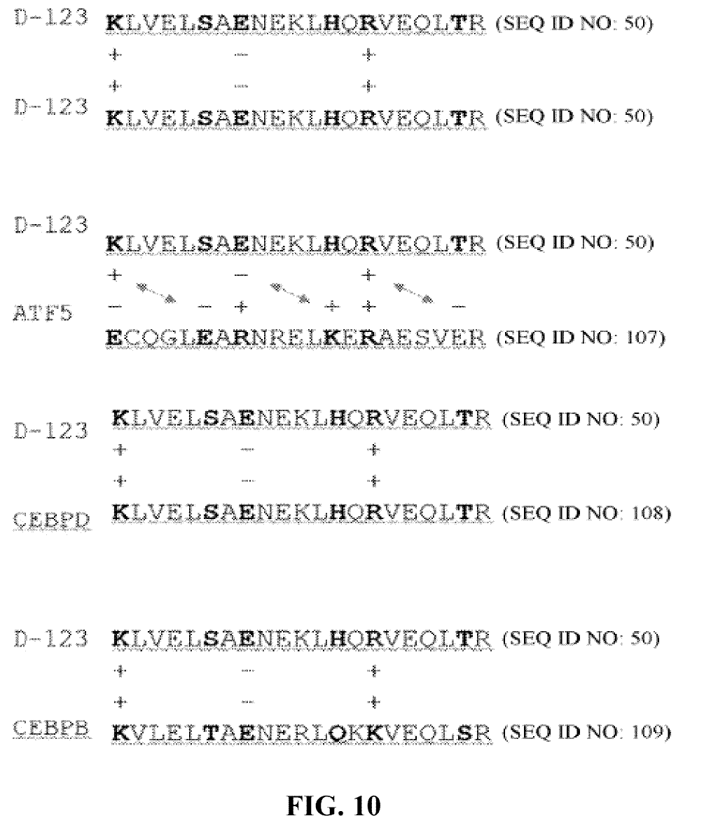

FIG. 10 shows alignments between the sequences of the D-123 (SEQ ID NO: 50) peptide with itself and with corresponding segments from ATF5 (ECQGLEARNRELKERAESVER; SEQ ID NO: 107), CEBPD (KLVELSAENEKLHQRVEQLTR; SEQ ID NO: 108), and CEBPB (KVLELTAENERLQKKVEQLSR; SEQ ID NO: 109). Residues that participate in i+5 interactions are bolded and their respective charges noted, if applicable. D-123 homodimers and dimers between D-123 and CEBPD or CEBPB show no attractive or repulsive i+5 charge interactions. Dimers between D-123 and ATF5 show three attractive charge interactions (indicated as “+3”) between the first, eighth, and 15^{th} residue of D-123 and the corresponding i+5 residue of ATF5 (indicated by pointed arrows).

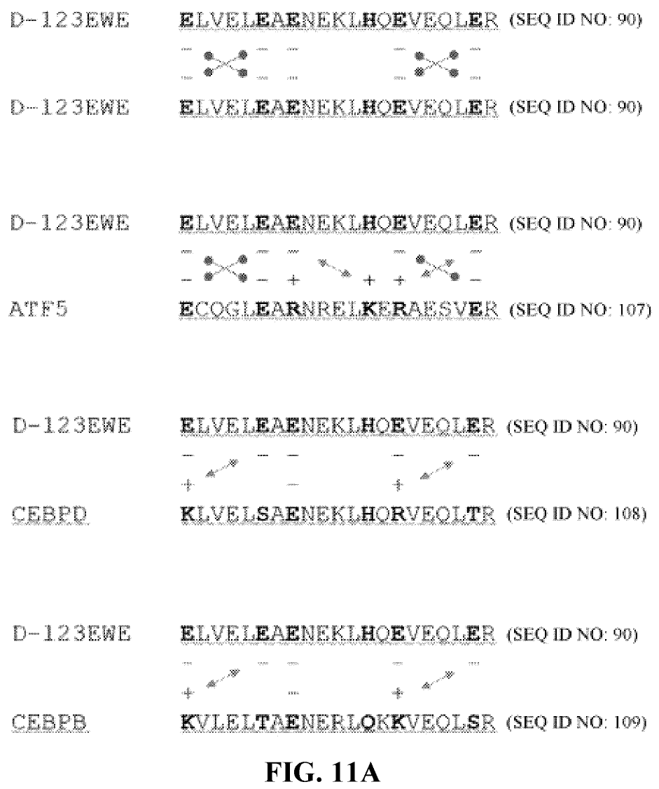

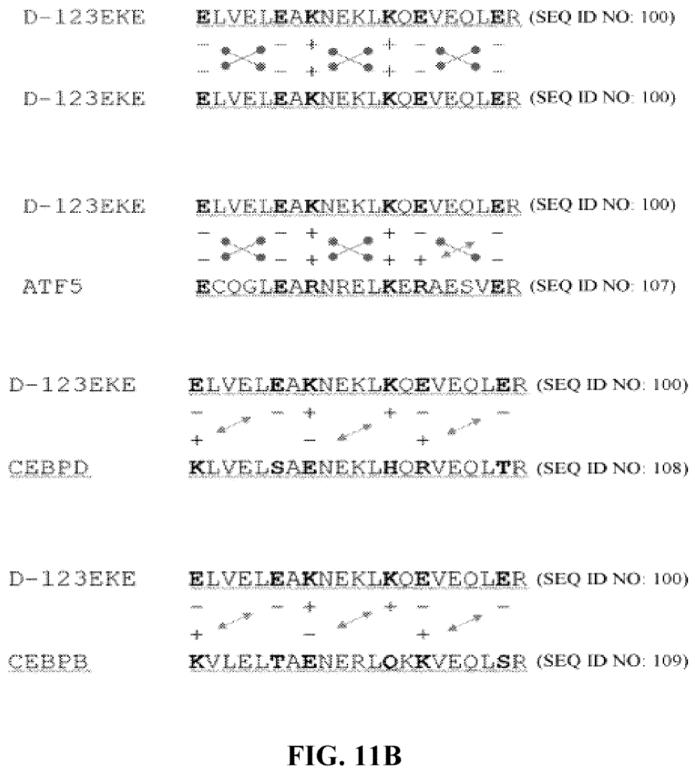

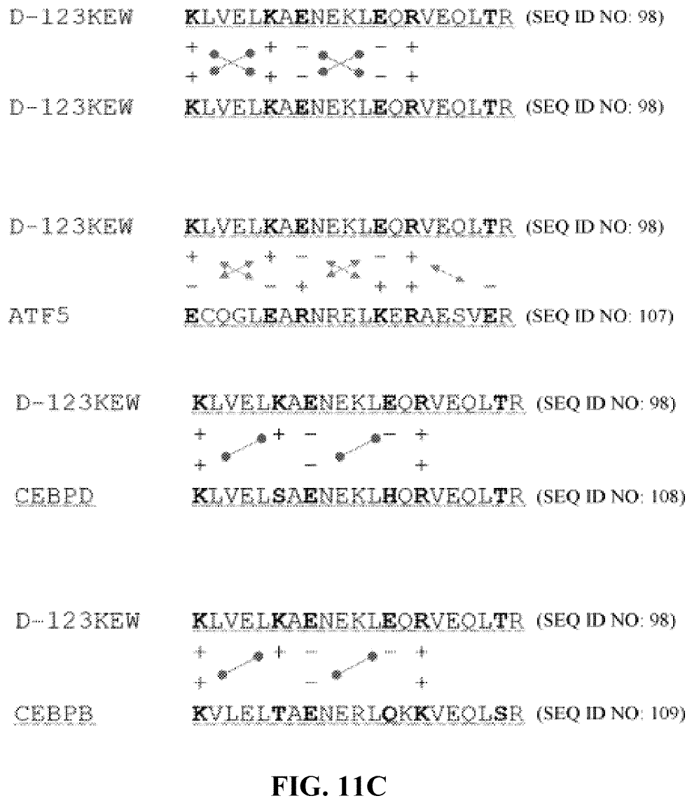

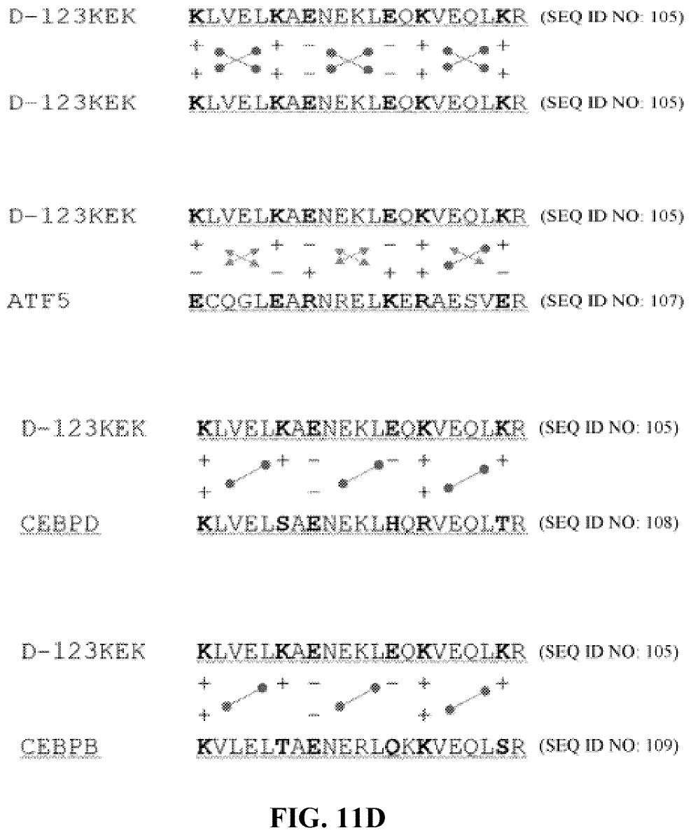

FIG. 11A shows alignments between the sequences of the D-123EWE (SEQ ID NO: 90) peptide with itself and with corresponding segments from ATF5, CEBPD, and CEBPB. Residues that participate in i+5 interactions are bolded and their respective charges noted, if applicable. As indicated, D-123EWE homodimers show four repulsive i+5 charge interactions (indicated by rounded arrows); dimers between D-123EWE and ATF5 show three repulsive and two attractive i+5 charge interactions; and dimers between D-123EWE and CEBPD or CEBPB each show two attractive i+5 charge interactions. Based on these i+5 charge interactions, the substitutions in D-123EWE are expected to disfavor homodimer formation, favor dimerization with CEBPB and CEBPD, and have a mixed effect on dimerization with ATF5.

FIG. 11B shows alignments between the sequences of the D-123EKE (SEQ ID NO: 100) peptide with itself and with corresponding segments from ATF5, CEBPD, and CEBPB. Residues that participate in i+5 interactions are bolded and their respective charges noted, if applicable. As indicated, D-123EKE homodimers show six repulsive i+5 charge interactions; dimers between D-123EKE and ATF5 show five repulsive and one attractive i+5 charge interactions; and dimers between D-123EKE and CEBPD or CEBPB each show three attractive i+5 charge interactions. Based on these i+5 charge interactions, the substitutions in D-123EKE are expected to disfavor homodimer formation, favor dimerization with CEBPB and CEBPD, and disfavor dimerization with ATF5.

FIG. 11C shows alignments between the sequences of the D-123KEW (SEQ ID NO: 98) peptide with itself and with corresponding segments from ATF5, CEBPD, and CEBPB. Residues that participate in i+5 interactions are bolded and their respective charges noted, if applicable. As indicated, D-123KEW homodimers show four repulsive i+5 charge interactions; dimers between D-123KEW and ATF5 show five attractive i+5 charge interactions; and dimers between D-123KEW and CEBPD or CEBPB each show two repulsive i+5 charge interactions. Based on these i+5 charge interactions, the substitutions in D-123KEW are expected to disfavor homodimer formation, disfavor dimerization with CEBPB and CEBPD, and favor dimerization with ATF5.

FIG. 11D shows alignments between the sequences of the D-123KEK (SEQ ID NO: 105) peptide with itself and with corresponding segments from ATF5, CEBPD, and CEBPB. Residues that participate in i+5 interactions are bolded and their respective charges noted, if applicable. As indicated, D-123KEK homodimers show six repulsive i+5 charge interactions; dimers between D-123KEK and ATF5 show one repulsive and five attractive i+5 charge interactions; and dimers between D-123KEK and CEBPD or CEBPB each show three repulsive i+5 charge interactions. Based on these i+5 charge interactions, the substitutions in D-123KEK are expected to disfavor homodimer formation, disfavor dimerization with CEBPB and CEBPD, and favor dimerization with ATF5.

Table 8 summarizes the results of analyses of i+5 interactions in homodimers and dimers with CEBPB/CEBPD or ATF5 for each of the D-123 variants listed in Table 5. Attractive i+5 charge interactions are indicated as +N and repulsive i+5 charge interactions as −N, where N indicates the number of attractive or repulsive i+5 charge interactions, respectively.

TABLE 8
Attractive and repulsive i + 5 interactions

| Construct | Homodimer | CEBPB/CEBPD | ATF5 |
|---|---|---|---|
| D-123 | 0 | 0 | +3 |
| D-123WWK | −2 | −1 | +3 |
| D-123WWE | −2 | +1 | +3, −1 |
| D-123WKW | −2 | +1 | +2, −2 |
| D-123WEW | −2 | −1 | +4 |
| D-123KWW | −2 | −1 | +4 |
| D-123EWW | −2 | +1 | +2, −2 |
| D-123WKK | −4 | +1, −1 | +2, −2 |
| D-123WEE | −4 | +1, −2 | +3, −1 |
| D-123KWK | −4 | −2 | +4 |
| D-123EWE | −4 | +2 | +2, −3 |
| D-123KKW | −4 | +1, −1 | +3, −2 |
| D-123EEW | −4 | +1, −1 | +3, −2 |
| D-123EKW | −4 | +2 | +1, −4 |
| D-123EWK | −4 | +1, −1 | +2, −2 |
| D-123WEK | −4 | −2 | +4 |
| D-123WKE | −4 | +2 | +2, −3 |
| D-123KWE | −4 | +1, −1 | +4, −1 |
| D-123KEW | −4 | −2 | +5 |
| D-123KEE | −6 | +1, −2 | +5, −1 |
| D-123EKE | −6 | 3 | +1, −5 |
| D-123EEK | −6 | +1, −2 | +3, −2 |
| D-123EEE | −6 | +2, −1 | +2, −3 |
| D-123KKK | −6 | +1, −2 | +3, −2 |
| D-123KKE | −6 | +2, −1 | +3, −3 |
| D-123KEK | −6 | −3 | +5, −1 |
| D-123EKK | −6 | +2, −1 | +1, −4 |

### Example 6—D-123EWE and D-123EKE Peptides Promote Apoptotic Death of HCT116 Cultures

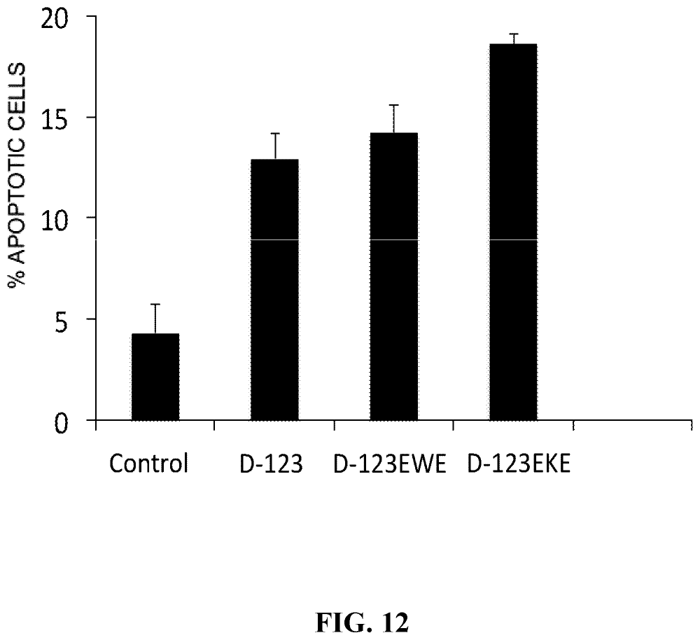

FIG. 12 is a graph reporting exemplary data showing D-123EWE and D-123EKE peptides promote apoptotic death of cancer cells. Replicate cultures of HCT116 cells were transfected with an empty DNA vector or DNA vectors expressing D-123, D-123EWE, or D-123EKE. Two days later, transfected cells were scored for proportion with apoptotic nuclei. Data are pooled from two independent experiments, each with n=4. Data represent means±SEM.

The data indicate that D-123EWE and D-123EKE peptides show activity comparable or greater to that of the D-123 peptide.

### Example 7—CP-D-123EKE Reduces Cell Numbers in Cancer Cell Lines

RQIKIWFQNRRMKWKELVELEAKNEKLKQEVEQLER (SEQ ID NO: 110) is an exemplary sequence of a construct referred to as CP-D-123EKE, wherein D-123EKE, italicized and bold, is preceded by a penetratin sequence. Peptide CP-D-123EKE was commercially synthesized.

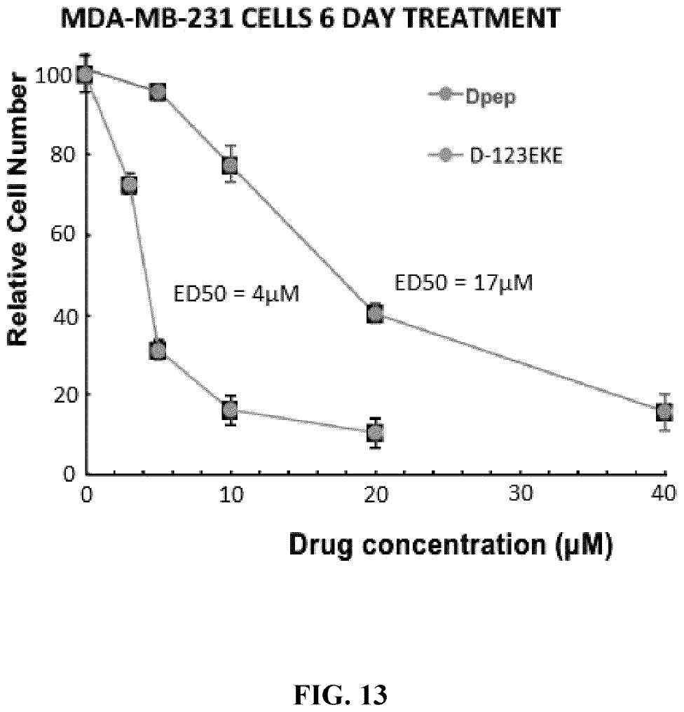

FIG. 13 is a graph reporting exemplary data showing CP-D-123EKE reduces cancer cell numbers. CP-D-123EKE and Dpep were diluted to the indicated concentrations in culture medium containing 2% fetal bovine serum and incubated with MDA-MB-231 cells for 6 days at which time cell counts were performed. Data are normalized to the number of cells in cultures maintained without peptide. Values represent mean±SEM (n=3 replicates). ED50s were calculated as the values at which the curves crossed the normalized relative cell number values of 50. The data indicate that CP-D-123EKE reduced cell count with a ED50 of 4 μM, while Dpep reduced cell count with an ED50 of 17 μM.

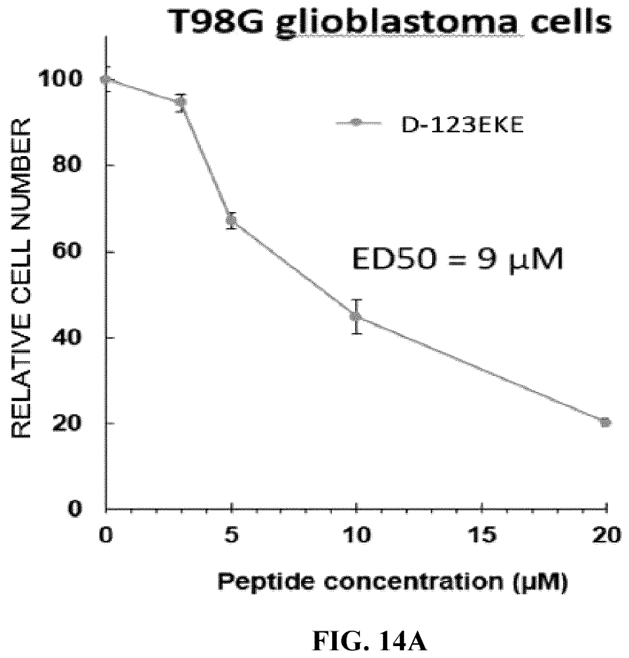

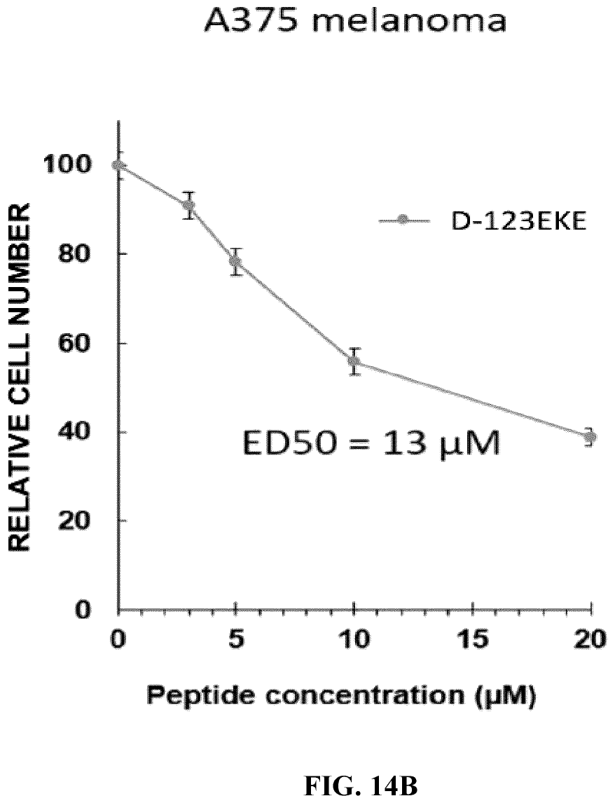

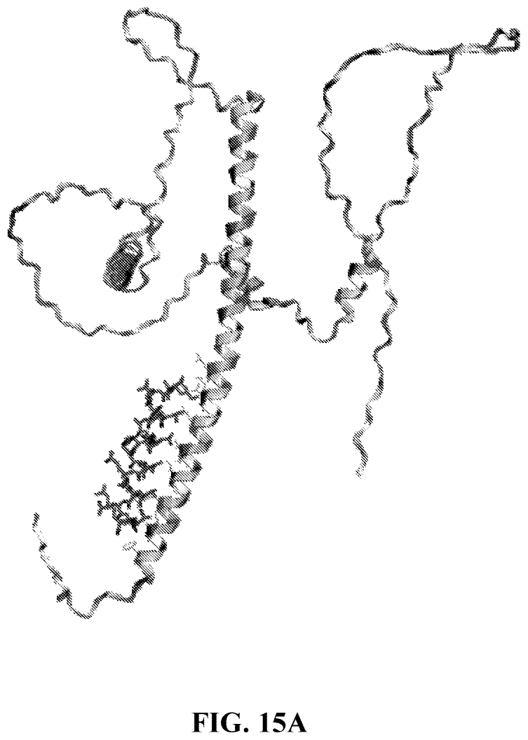

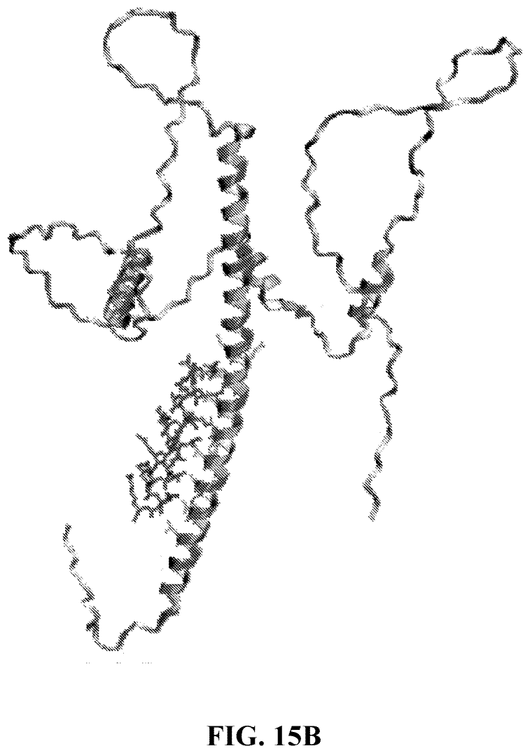

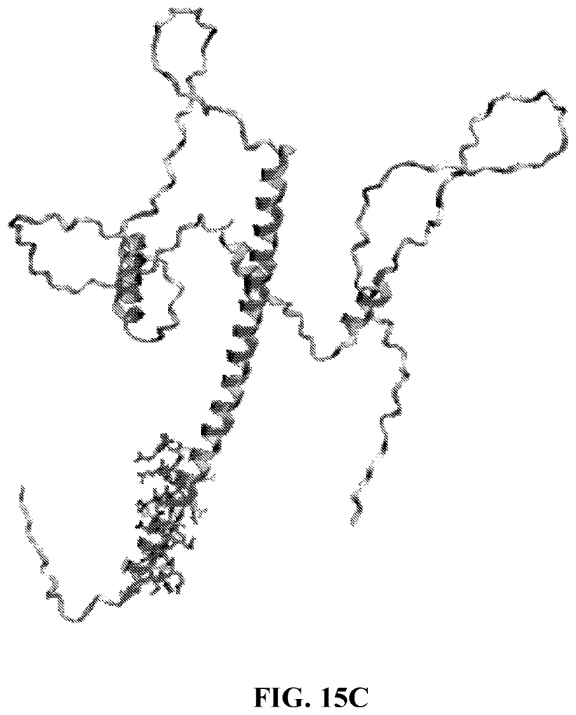

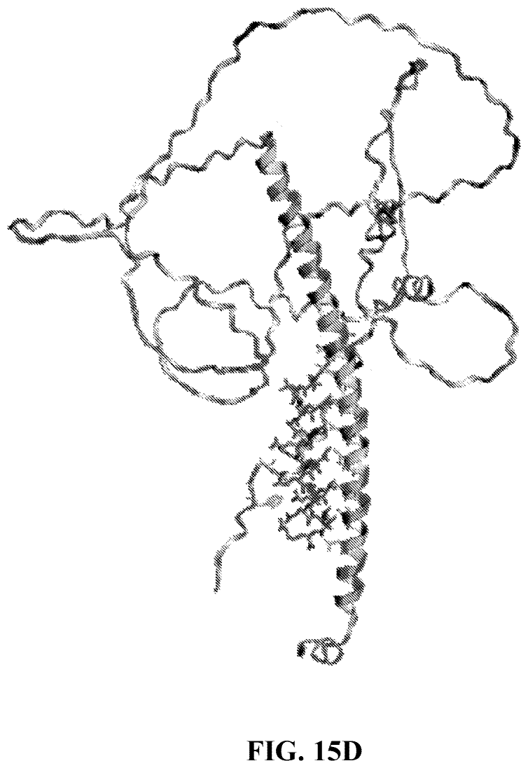

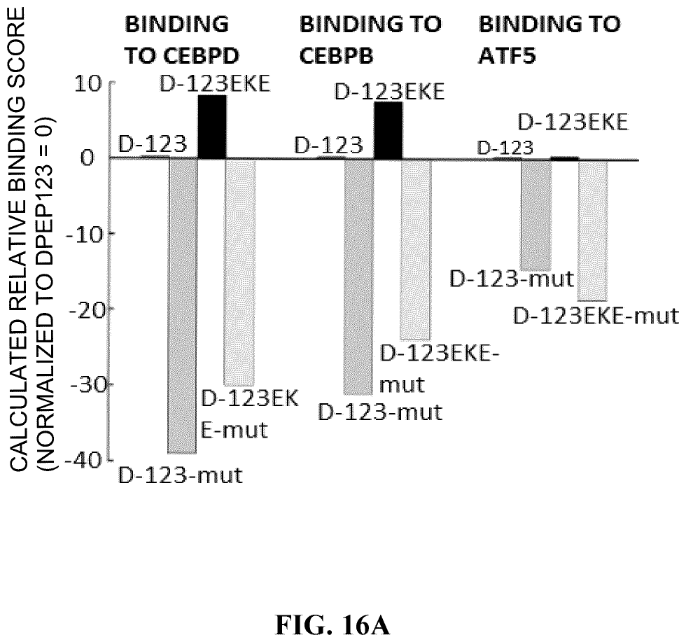

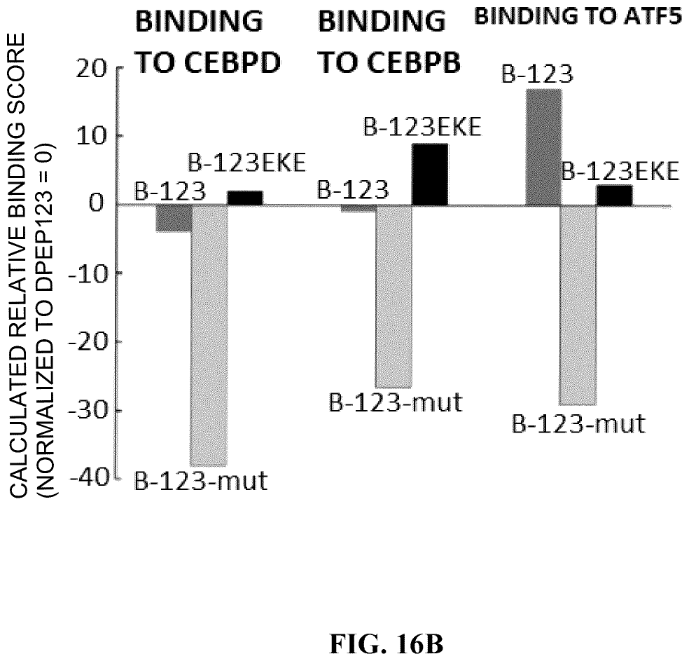

FIGS. 14A-14B are graphs reporting exemplary data showing CP-D-123EKE reduces cancer cell numbers. CP-D-123EKE was diluted to the indicated concentrations in culture medium containing 2% fetal bovine serum and incubated with T98G cells (14A) and A375 cells (14B) for 6 days at which time cell counts were performed. Data are normalized to the number of cells in cultures maintained without peptide. Values represent mean±SEM (n=3 replicates). ED50s were calculated as the values at which the curves crossed the normalized relative cell number values of 50. The data indicate that CP-D-123EKE reduced T98G cell count with a ED50 of 9 UM and A375 cell count with an ED50 of 13 μM.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

1. A dominant negative protein comprising a leucine zipper domain having 90% or greater homology to any one of SEQ ID NOs: 7-10, 50, 52, 54, 56, 58, or 81-106, or SEQ ID NOs: 3-6, 65, 67, 69, 71, 73, 75, or 77-80.

2. (canceled)

3. The dominant negative protein of claim 1, wherein a cell penetrating peptide is linked directly or indirectly to the leucine zipper domain, preferably wherein the cell penetrating peptide is penetratin 1, more preferably wherein the dominant negative protein consists essentially of any one of SEQ ID NOS: 51, 53, 55, 57 or 59-63, or SEQ ID NOS: 66, 68, 70, 72, 74, or 76-80.

4-10. (canceled)

11. A polynucleotide comprising a sequence encoding the dominant negative protein of claim 1.

12. A composition comprising the dominant negative protein of claim 1 and a pharmaceutically acceptable excipient.

13. The composition of claim 12, further comprising a chemotherapeutic.

14. A method of decreasing activity or viability of a neoplastic cell, comprising:
contacting the neoplastic cell with the dominant negative protein of claim 1 for a time and under conditions sufficient to cause a decrease in activity or viability of the neoplastic cell.

15. A method of treating cancer in a subject, comprising:
administering to the subject an effective amount of the dominant negative protein of claim 1 for a time sufficient to treat a cancer in a subject.

16. The method of claim 15, further comprising concurrently or during the same course of treatment administering gamma radiation or a chemotherapeutic to the subject, preferably wherein the chemotherapeutic comprises paclitaxel, chloroquine, doxorubicin, nab-paclitaxel, abraxane, docetaxel, 10-deacetylbaccatin III, baccatin III, paclitaxel C, 7-epipaclitaxel, daunorubicin, epirubicin, idarubicin, mitoxantrone, valrubicin, 5-Azacytidine (Azacitidine), 5-Aza-2′-deoxycytidine (Decitabine), Guadecitabine, or ABT263 (Navitoclax); and/or
wherein the method further comprises inhibiting metastasis of the cancer, inhibiting recurrence of the cancer from dormant cancer cells, or both, in the subject.

17-20. (canceled)

21. A non-naturally occurring composition comprising a peptide,
wherein the peptide comprises a CCAAT/enhancer-binding protein delta (CEBPD) leucine zipper domain comprising one to four CEBPD repeat portions,
wherein each CEBPD repeat portion has at least 70% sequence identity to any one of SEQ ID NOs: 7-10.

22. The composition of claim 21, wherein the peptide comprises two, three, or four CEBPD repeat portions;
wherein a CEBPD repeat portion has a different amino acid sequence from another CEBPD repeat portion in the domain, preferably wherein each CEBPD repeat portion has a different amino acid sequence from any other CEBPD repeat portion in the CEBPD leucine zipper domain;
wherein a CEBPD repeat portion has the same amino acid sequence as another CEBPD repeat portion in the CEBPD leucine zipper domain;
wherein the peptide comprises more than one CEBPD repeat portion and the portions are in tandem;
wherein at least one CEBPD repeat portion has an amino acid sequence of any one of SEQ ID NOs: 7-10 with an amino acid substitution at one or two positions;
wherein at least one CEBPD repeat portion has an amino acid sequence of any one of SEQ ID NOs: 7-10 with an amino acid substitution at the first position and/or sixth position;
wherein two separate molecules of the peptide form 1 to 6 repulsive charge interactions between their CEBPD repeat portions;
wherein a molecule of the peptide and a molecule of a protein comprising a wild-type Activating Transcription Factor 5 (ATF5) leucine zipper domain comprising SEQ ID NO: 107 form 1 to 5 attractive charge interactions between the CEBPD repeat portion of the peptide and the wild-type ATF5leucine zipper domain of the protein;
wherein a molecule of the peptide and a molecule of a protein comprising a wild-type CEBPD leucine zipper domain comprising SEQ ID NO: 108 form 1 to 5 attractive charge interactions between the CEBPD repeat portion of the peptide and the wild-type CEBPD leucine zipper domain of the protein;
wherein a molecule of the peptide and a molecule of a protein comprising a wild-type CCAAT/enhancer-binding protein beta (CEBPB) leucine zipper domain comprising SEQ ID NO: 109 form 1 to 5 attractive charge interactions between the CEBPD repeat portion of the peptide and the wild-type CEBPB leucine zipper domain of the protein;
wherein at least one CEBPD repeat portion has at least 70% sequence identity to any one of SEQ ID NOs: 7-10 and the fifth amino acid of the at least one CEBPD repeat portion is a leucine (L) residue;
wherein at least one CEBPD repeat portion has at least 70% sequence identity to SEQ ID NO: 7, and the first amino acid is other than a lysine (K) residue, preferably wherein the first amino acid of the at least one CEBPD repeat portion is a negatively charged amino acid, preferably an aspartic acid (D) residue or a glutamic acid (E) residue;
wherein at least one CEBPD repeat portion has at least 70% sequence identity to SEQ ID NO: 7, and the sixth amino acid is other than a serine(S) residue, preferably wherein the sixth amino acid of the at least one CEBPD repeat portion is a negatively charged amino acid, preferably an aspartic acid (D) residue or a glutamic acid (E) residue, or a positively charged amino acid, preferably a lysine (K) residue or an arginine (R) residue;
wherein at least one CEBPD repeat portion has at least 70% sequence identity to SEQ ID NO: 8, and the first amino acid is other than a glutamic acid (E) residue, preferably wherein the first amino acid of the at least one CEBPD repeat portion is a positively charged amino acid, preferably a lysine (K) residue or an arginine (R) residue;
wherein at least one CEBPD repeat portion has at least 70% sequence identity to SEQ ID NO: 8, and the sixth amino acid is other than a histidine (H) residue, preferably wherein the sixth amino acid of the at least one CEBPD repeat portion is a negatively charged amino acid, preferably an aspartic acid (D) residue or a glutamic acid (E) residue, or a positively charged amino acid, preferably a lysine (K) residue or an arginine (R) residue;
wherein at least one CEBPD repeat portion has at least 70% sequence identity to SEQ ID NO: 9, and the first amino acid is other than an arginine (R) residue, preferably wherein the first amino acid of the at least one CEBPD repeat portion is a negatively charged amino acid, preferably an aspartic acid (D) residue or a glutamic acid (E) residue, or a positively charged amino acid, preferably a lysine (K) residue or an arginine (R) residue;
at least one CEBPD repeat portion has at least 70% sequence identity to SEQ ID NO: 9, and the sixth amino acid is other than a threonine (T) residue, preferably wherein the sixth amino acid of the at least one CEBPD repeat portion is a negatively charged amino acid, preferably an aspartic acid (D) residue or a glutamic acid (E) residue, or a positively charged amino acid, preferably a lysine (K) residue or an arginine (R) residue;
wherein the CEBPD leucine zipper domain has at least 90% sequence identity to any one of SEQ ID NOs: 7-10, 50, 52, 54, 56, 58, or 81-106
wherein the peptide further comprises a cell-penetrating peptide portion, preferably wherein the cell-penetrating peptide portion is linked directly or indirectly to the CEBPD leucine zipper domain, preferably wherein the cell-penetrating peptide portion comprises a RL16 sequence or variant thereof, penetratin 1 sequence or variant thereof, transportan sequence or variant thereof, pIS1 sequence or variant thereof, Tat (48-60) sequence or variant thereof, pVEC sequence or variant thereof, a model amphipathic peptide (MAP) or variant thereof, or a membrane translocating sequence (MTS) or a variant thereof, preferably wherein the cell-penetrating peptide portion is N-terminal of the CEBPD leucine zipper domain;
wherein the peptide is a truncated dominant negative CEBPD protein;
wherein the peptide is non-naturally occurring;
wherein the CEBPD leucine zipper domain is 7-35, 7-28, 7-21, or 7-14 amino acids in length;
wherein the peptide is 7-100, 7-75, 7-50, or 7-40 amino acids in length; and/or
wherein the peptide comprises SEQ ID NOs: 51, 53, 55, 57 or 59-63.

23-54. (canceled)

55. A non-naturally occurring composition comprising a peptide,
wherein the peptide comprises a CCAAT/enhancer-binding protein beta (CEBPB) leucine zipper domain comprising one to four CEBPB repeat portions,
wherein each CEBPB repeat portion has at least 70% sequence identity to any one of SEQ ID NOS: 3-6.

56. The composition of claim 55, wherein the peptide comprises two, three, or four CEBPB repeat portions;
wherein a CEBPB repeat portion has a different amino acid sequence from another CEBPB repeat portion in the domain, preferably wherein each CEBPB repeat portion has a different amino acid sequence from any other CEBPB repeat portion in the CEBPB leucine zipper domain;
wherein a CEBPB repeat portion has the same amino acid sequence as another CEBPB repeat portion in the CEBPB leucine zipper domain;
wherein the peptide comprises more than one CEBPB repeat portion and the portions are in tandem;
wherein at least one CEBPB repeat portion has an amino acid sequence of any one of SEQ ID NOs: 3-6 with an amino acid substitution at one or two positions;
wherein at least one CEBPB repeat portion has an amino acid sequence of any one of SEQ ID NOS: 3-6 with an amino acid substitution at the first position and/or sixth position;
wherein two separate molecules of the peptide form 1 to 6 repulsive charge interactions between their CEBPB repeat portions;
wherein a molecule of the peptide and a molecule of a protein comprising a wild-type Activating Transcription Factor 5 (ATF5) leucine zipper domain comprising SEQ ID NO: 107 form 1 to 5 attractive charge interactions between the CEBPB repeat portion of the peptide and the wild-type ATF5 leucine zipper domain of the protein;
wherein a molecule of the peptide and a molecule of a protein comprising a wild-type CEBPD leucine zipper domain comprising SEQ ID NO: 108 form 1 to 5 attractive charge interactions between the CEBPB repeat portion of the peptide and the wild-type CEBPD leucine zipper domain of the protein;
wherein a molecule of the peptide and a molecule of a protein comprising a wild-type CCAAT/enhancer-binding protein beta (CEBPB) leucine zipper domain comprising SEQ ID NO: 109 form 1 to 5 attractive charge interactions between the CEBPB repeat portion of the peptide and the wild-type CEBPB leucine zipper domain of the protein;
wherein at least one CEBPB repeat portion has at least 70% sequence identity to any one of SEQ ID NOs: 3-6 and the fifth amino acid of the at least one CEBPB repeat portion is a leucine (L) residue;
wherein at least one CEBPB repeat portion has at least 70% sequence identity to SEQ ID NO: 3, and the first amino acid is other than a lysine (K) residue, preferably wherein the first amino acid of the at least one CEBPB repeat portion is a negatively charged amino acid, preferably an aspartic acid (D) residue or a glutamic acid (E) residue;
wherein at least one CEBPB repeat portion has at least 70% sequence identity to SEQ ID NO: 3, and the sixth amino acid is other than a threonine (T) residue, preferably wherein the sixth amino acid of the at least one CEBPB repeat portion is a negatively charged amino acid, preferably an aspartic acid (D) residue or a glutamic acid (E) residue, or a positively charged amino acid, preferably a lysine (K) residue or an arginine (R) residue;
wherein at least one CEBPB repeat portion has at least 70% sequence identity to SEQ ID NO: 4, and the first amino acid is other than a glutamic acid (E) residue, preferably wherein the first amino acid of the at least one CEBPB repeat portion is a positively charged amino acid, preferably a lysine (K) residue or an arginine (R) residue;
wherein at least one CEBPB repeat portion has at least 70% sequence identity to SEQ ID NO: 4, and the sixth amino acid is other than a glutamine (Q) residue, preferably wherein the sixth amino acid of the at least one CEBPB repeat portion is a negatively charged amino acid, preferably an aspartic acid (D) residue or a glutamic acid (E) residue, or a positively charged amino acid, preferably a lysine (K) residue or an arginine (R) residue;
wherein at least one CEBPB repeat portion has at least 70% sequence identity to SEQ ID NO: 5, and the first amino acid is other than a lysine (K) residue, preferably wherein the first amino acid of the at least one CEBPB repeat portion is a negatively charged amino acid, preferably an aspartic acid (D) residue or a glutamic acid (E) residue, or a positively charged amino acid, preferably a lysine (K) residue or an arginine (R) residue;
wherein at least one CEBPB repeat portion has at least 70% sequence identity to SEQ ID NO: 5, and the sixth amino acid is other than a serine(S) residue, preferably wherein the sixth amino acid of the at least one CEBPB repeat portion is a negatively charged amino acid, preferably an aspartic acid (D) residue or a glutamic acid (E) residue, or a positively charged amino acid, preferably a lysine (K) residue or an arginine (R) residue;
wherein the CEBPB leucine zipper domain has at least 90% sequence identity to any one of SEQ ID NOS: 3-6, 65, 67, 69, 71, 73, 75, 77-80, or 114;
wherein the peptide further comprises a cell-penetrating peptide portion, preferably wherein the cell-penetrating peptide portion is linked directly or indirectly to the CEBPB leucine zipper domain, preferably wherein the cell-penetrating peptide portion comprises a RL16 sequence or variant thereof, penetratin 1 sequence or variant thereof, transportan sequence or variant thereof, pIS1 sequence or variant thereof, Tat (48-60) sequence or variant thereof, pVEC sequence or variant thereof, a model amphipathic peptide (MAP) or variant thereof, or a membrane translocating sequence (MTS) or a variant thereof, preferably wherein the cell-penetrating peptide portion is N-terminal of the CEBPB leucine zipper domain;
wherein the peptide is a truncated dominant negative CEBPB protein;
wherein the peptide is non-naturally occurring;
wherein the CEBPB leucine zipper domain is 7-35, 7-28, 7-21, or 7-14 amino acids in length;
wherein the peptide is 7-100, 7-75, 7-50, or 7-40 amino acids in length; and/or
wherein the peptide comprises SEQ ID NOs: 66, 68, 70, 72, 74, 76-80, or 114.

57-88. (canceled)

89. The composition of claim 21, further comprising a pharmaceutically acceptable excipient and/or a chemotherapeutic agent, preferably wherein the chemotherapeutic agent comprises paclitaxel, chloroquine, doxorubicin, nab-paclitaxel, abraxane, docetaxel, 10-deacetylbaccatin III, baccatin III, paclitaxel C, 7-epipaclitaxel, daunorubicin, epirubicin, idarubicin, mitoxantrone, valrubicin, 5-Azacytidine (Azacitidine), 5-Aza-2′-deoxycytidine (Decitabine), Guadecitabine, or ABT263 (Navitoclax).

90. (canceled)

91. (canceled)

92. A method of decreasing activity or viability of a neoplastic cell, comprising delivering the composition of claim 21 to the neoplastic cell, preferably wherein the neoplastic cell is a cancer cell, preferably a cancer cell, preferably breast cancer cell, colon cancer cell, prostate cancer cell, bladder cancer cell, soft-tissue sarcoma cell, an advanced lung cancer cell, lung cancer cell, non-small cell lung cancer cell, small cell lung cancer cell, mesothelioma cell, esophageal cancer cell, liver cancer cell, renal cell cancer cell, melanoma cell, skin cancer cell, basal cell skin cancer cell, squamous cell skin cancer cell, or a squamous cell carcinoma cell.

93. (canceled)

94. A method of treating cancer in a subject, comprising administering to the subject an effective amount of the composition of claim 21, preferably wherein the method further comprises inhibiting metastasis of the cancer, inhibiting recurrence of the cancer from dormant cancer cells, or both, in the subject, preferably wherein the cancer is a breast cancer, estrogen receptor positive (ER+) breast cancer, triple-negative breast cancer (TNBC), colon cancer, prostate cancer, bladder cancer, soft-tissue sarcoma, an advanced lung cancer, lung cancer, non-small cell lung cancer, small cell lung cancer, mesothelioma, esophageal cancer, liver cancer, renal cell cancer, melanoma, skin cancer, basal cell skin cancer, squamous cell skin cancer, squamous cell carcinoma of the head and neck, or leukemia.

95. (canceled)

96. (canceled)

97. A polynucleotide molecule encoding the peptide of claim 21.

98. (canceled)

99. (canceled)

100. The composition of claim 55, further comprising a pharmaceutically acceptable excipient.

101. A method of decreasing activity or viability of a neoplastic cell, comprising delivering the composition of claim 55 to the neoplastic cell.

102. A method of treating cancer in a subject, comprising administering to the subject an effective amount of the composition of claim 55.

103. A polynucleotide molecule encoding the peptide of claim 55.
